SEQUENCE LISTING (19 sequences)

SEQ ID NO: 1 - DNA, 1839 bp, Mus musculus
ggcacgagggaggaagcgccgccgggtccgctctgctctgggtccggctgggccatggagtccatgtctgagctcgcgccccgctgcctcttatttcctttgctgctgctgcttccgctgctgctccttcctgccccgaagctaggcccgagtcccgccggggctgaggagaccgactgggtgcgattgcccagcaaatgcgaagtgtgcaagtatgttgctgtggagctgaagtcggcttttgaggaaacgggaaagaccaaggaagtgattgacaccggctatggcatcctggacgggaagggctctggagtcaagtacaccaagtcggacttacggttaattgaagtcactgagaccatttgcaagaggcttctggactacagcctgcacaaggagaggactggcagcaaccggtttgccaagggtatgtcggagacctttgagacgctgcacaacctagtccacaaaggggtcaaggtggtgatggatatcccctatgagctgtggaacgagacctcagcagaggtggctgacctcaagaagcagtgtgacgtgctggtggaagagtttgaagaggtgattgaggactggtacaggaaccaccaggaggaagacctgactgaattcctctgtgccaaccacgtgctgaagggaaaggacacgagttgcctagcagagcggtggtctggcaagaagggggacatagcctccctgggagggaagaaatccaagaagaagcgcagcggagtcaagggctcctccagtggcagcagcaagcagaggaaggaactggggggcctgggggaggatgccaacgccgaggaggaggagggtgtgcagaaggcatcgcccctcccacacagcccccctgatgagctgtgagcccagcttagtgtccttgaatcaagacccctgacttcagagcttgggacacgcacagcgcagcgcagcgcagctccagcaaggacagctgctgtccagcatcaggtctcctcccttggctgtgcccctttccttcccttgaacaacagcaagaggtggaaggatctggggtgctgggagacggcaccccaaagggaagaggaggaggagcagaaggcagctctctttctacacagtccccctcacgagctccggggtccacccagcatccccaggctgagatccaggctcctgacatggaagctgaagagcatgaggcacataagatgctcaccagcgcccccttcagccaggaaggactccgtgcagcctcagcagccaggcctgcctcttccttccaccaagcattctcttctgctggtccttgtcggatggtaaattcgagaacttccaggacaaactcgggtgtggcacaaaggggctggacgccagagccagagccacgccagagactgcagagagggcacctgacctaacccccctggaaagccaatctgcagttcccgtgtccacccactcctcctgaggacgcctcatgctctgcccagcccttctcccagggctaccagagtaaacaccttttggcctttcggtttggttcctgggtcctcatcagcctccagagtgtcccctcatcgatcttttttgcctttgtcccccaatcccaggggctggaaggccatcaccatcattggaggcttaacctgtcagttactaggaggtgctgggagcgcccggggttggtttggggtaatcactcactggctctcagccttctaacactgcagccccttaatacagttccttctgttgtggtgactcccacgcccccacacacacaccataaaattatttcgatgctgtttcataactgtaaaaaaaaaaaaaaaaaaa

SEQ ID NO: 2 - DNA, 1362 bp, Homo sapiens
cgagccatggattcaatgcctgagcccgcgtcccgctgtcttctgcttcttcccttgctgctgctgctgctgctgctgctgccggccccggagctgggcccgagccaggccggagctgaggagaacgactgggttcgcctgcccagcaaatgcgaagtgtgtaaatatgttgctgtggagctgaagtcagcctttgaggaaaccggcaagaccaaggaggtgattggcacgggctatggcatcctggaccagaaggcctctggagtcaaatacaccaagtcggacttgcggttaatcgaagtcactgagaccatttgcaagaggctcctggattatagcctgcacaaggagaggaccggcagcaatcgatttgccaagggcatgtcagagacctttgagacattacacaacctggtacacaaaggggtcaaggtggtgatggacatcccctatgagctgtggaacgagacttctgcagaggtggctgacctcaagaagcagtgtgatgtgctggtggaagagtttgaggaggtgatcgaggactggtacaggaaccaccaggaggaagacctgactgaattcctctgcgccaaccacgtgctgaagggaaaagacaccagttgcctggcagagcagtggtccggcaagaagggagacacagctgccctgggagggaagaagtccaagaagaagagcagcagggccaaggcagcaggcggcaggagtagcagcagcaaacaaaggaaggagctgggtggccttgagggagaccccagccccgaggaggatgagggcatccagaaggcatcccctctcacacacagcccccctgatgagctctgagcccacccagcatcctctgtcctgagacccctgattttgaagctgaggagtcaggggcatggctctggcaggccgggatggccccgcagccttcagcccctccttgccttggctgtgccctcttctgccaaggaaagacacaagccccaggaagaactcagagccgtcatgggtagcccacgccgtcctttcccctccccaagtgtttctctcctgacccagggttcaggcaggccttgtggtttcaggactgcaaggactccagtgtgaactcaggaggggcaggtgtcagaactgggcaccaggactggagccccctccggagaccaaactcaccatccctcagtcctccccaacagggtactaggactgcagccccctgtagctcctctctgcttacccctcctgtggacaccttgcactctgcctggcccttcccagagcccaaagagtaaaaatgttctggttctgaaaaaaaaaaaaaaaaaaaaaaaaaaaaaaaaaaaaaaaaaaaaaaaa

SEQ ID NO: 3 - protein, 276 aa, Mus musculus
MESMSELAPRCLLFPLLLLLPLLLLPAPKLGPSPAGAEETDWVRLPSKCEVCKYVAVELKSAFEETGKTKEVIDTGYGILDGKGSGVKYTKSDLRLIEVTETICKRLLDYSLHKERTGSNRFAKGMSETFETLHNLVHKGVKVVMDIPYELWNETSAEVADLKKQCDVLVEEFEEVIEDWYRNHQEEDLTEFLCANHVLKGKDTSCLAERWSGKKGDIASLGGKKSKKKRSGVKGSSSGSSKQRKELGGLGEDANAEEEEGVQKASPLPHSPPDEL

SEQ ID NO: 4 - protein, 278 aa, Homo sapiens [sequence omitted]

SEQ ID NO: 5 - DNA, 25 bp, Artificial sequence
  Primer
agctcagacatggactccatggccc

SEQ ID NO: 6 - DNA, 25 bp, Artificial sequence
  Primer
tgcgattgcccagcaaatgcgaagt

SEQ ID NO: 7 - DNA, 49 bp, Artificial sequence
  Primer
ctggttcttgtcggcttggcccaaagctcagacatggactccatggccc

SEQ ID NO: 8 - DNA, 49 bp, Artificial sequence
  Primer
ggtcctcgctctgtgtccgttgaatgcgattgcccagcaaatgcgaagt

SEQ ID NO: 9 - DNA, 25 bp, Artificial sequence
  Primer
gggccatggagtccatgtctgagct

SEQ ID NO: 10 - DNA, 25 bp, Artificial sequence
  Primer
acttcgcatttgctgggcaatcgca

SEQ ID NO: 11 - DNA, 471 bp, Artificial sequence
  PCR amplification product
  misc_feature (260)..(260): n is a, c, g, or t
acagaaaacaagaaacaaaaaccatgaaagatagtctgttatccagggctagaatgcccaaggctggttcatccaaggtatgatgaaggttcacccgctaggaactgatgctccagctactgagcctcctttagctggcagtgatatcgctatagggcgccaaagccaccatccgctctctgattgggtgagatgggaaaaaaaaaagatagttcctctcattggctataaagcagacgccgagcgaacccattggttgngtcgcccgcgggccttggtcggtttcgcaagccgctagaggctaccgggcgaggggcgggccggagctcgccgttgccgtggttacccagagacacgtgcgcagtcccggaagcggccgggggaagctgctccgcgcgcgctgccggaggaagcgccgccgggtccgctctgctctgggtccggctgggccatggagtccatgtctgagct

SEQ ID NO: 12 - DNA, 370 bp, Artificial sequence
  PCR amplification product
tgcgattgcccagcaaatgcgaaggtgagggggcggggccgcggggcgtagccaagcccgaggggcgggagggggcggggcctgtgggaagggtctgggcctggcaggacctgggctggggtctccttggccctgctgtgtgctttgcggcaatgctgggtgctgtgactctcggataacctggagatccctgcttttgggcgaatccgggggtagttgctcatcaagactagaggtgggggtggagggaaggcttcatacaggaagcctgctgcgaaatgaagagttggccagggaaagcatggcgtgcagaggaactcactccgcagaaaccacagaaacagaggcagatgaggacgccctgccggcc

SEQ ID NO: 13 - DNA, 107 bp, Artificial sequence
  Deleted gene fragment
cgcgccccgctgcctcttatttcctttgctgctgctgcttccgctgctgctccttcctgccccgaagctaggcccgagtcccgccggggctgaggagaccgactggg

SEQ ID NO: 14 - DNA, 1848 bp, Artificial sequence
  Expanded T243 gene
ggcacgagggaggaagcgccgccgggtccgctctgctctgggtccggctgggccatggagtccatgtctgagctgctgctgctgctgctgctgctgctgctgctgctgctgctgctgctgctgctgctgctgctgctgctgctgctgctgctgctgctgctgctgctgctgctgctgctgctgctgctgctgcgattgcccagcaaatgcgaagtgtgcaagtatgttgctgtggagctgaagtcggcttttgaggaaacgggaaagaccaaggaagtgattgacaccggctatggcatcctggacgggaagggctctggagtcaagtacaccaagtcggacttacggttaattgaagtcactgagaccatttgcaagaggcttctggactacagcctgcacaaggagaggactggcagcaaccggtttgccaagggtatgtcggagacctttgagacgctgcacaacctagtccacaaaggggtcaaggtggtgatggatatcccctatgagctgtggaacgagacctcagcagaggtggctgacctcaagaagcagtgtgacgtgctggtggaagagtttgaagaggtgattgaggactggtacaggaaccaccaggaggaagacctgactgaattcctctgtgccaaccacgtgctgaagggaaaggacacgagttgcctagcagagcggtggtctggcaagaagggggacatagcctccctgggagggaagaaatccaagaagaagcgcagcggagtcaagggctcctccagtggcagcagcaagcagaggaaggaactggggggcctgggggaggatgccaacgccgaggaggaggagggtgtgcagaaggcatcgcccctcccacacagcccccctgatgagctgtgagcccagcttagtgtccttgaatcaagacccctgacttcagagcttgggacacgcacagcgcagcgcagcgcagctccagcaaggacagctgctgtccagcatcaggtctcctcccttggctgtgcccctttccttcccttgaacaacagcaagaggtggaaggatctggggtgctgggagacggcaccccaaagggaagaggaggaggagcagaaggcagctctctttctacacagtccccctcacgagctccggggtccacccagcatccccaggctgagatccaggctcctgacatggaagctgaagagcatgaggcacataagatgctcaccagcgcccccttcagccaggaaggactccgtgcagcctcagcagccaggcctgcctcttccttccaccaagcattctcttctgctggtccttgtcggatggtaaattcgagaacttccaggacaaactcgggtgtggcacaaaggggctggacgccagagccagagccacgccagagactgcagagagggcacctgacctaacccccctggaaagccaatctgcagttcccgtgtccacccactcctcctgaggacgcctcatgctctgcccagcccttctcccagggctaccagagtaaacaccttttggcctttcggtttggttcctgggtcctcatcagcctccagagtgtcccctcatcgatcttttttgcctttgtcccccaatcccaggggctggaaggccatcaccatcattggaggcttaacctgtcagttactaggaggtgctgggagcgcccggggttggtttggggtaatcactcactggctctcagccttctaacactgcagccccttaatacagttccttctgttgtggtgactcccacgcccccacacacacaccataaaattatttcgatgctgtttcataactgtaaaaaaaaaaaaaaaaaaa

SEQ ID NO: 15 - protein, 279 aa, Artificial sequence
  Expanded T243 expression product
MESMSELLLLLLLLLLLLLLLLLLLLLLLLLLLLLLLLLLLLLLLLRLPSKCEVCKYVAVELKSAFEETGKTKEVIDTGYGILDGKGSGVKYTKSDLRLIEVTETICKRLLDYSLHKERTGSNRFAKGMSETFETLHNLVHKGVKVVMDIPYELWNETSAEVADLKKQCDVLVEEFEEVIEDWYRNHQEEDLTEFLCANHVLKGKDTSCLAERWSGKKGDIASLGGKKSKKKRSGVKGSSSGSSKQRKELGGLGEDANAEEEEGVQKASPLPHSPPDEL

SEQ ID NO: 16 - DNA, 1909 bp, Mus musculus
  misc_feature (1650)..(1650): n is a, c, g, or t
  misc_feature (1673)..(1673): n is a, c, g, or t
  misc_feature (1677)..(1677): n is a, c, g, or t
  misc_feature (1738)..(1738): n is a, c, g, or t
  misc_feature (1756)..(1756): n is a, c, g, or t
cgttgctgtcgggccgggggaagctgctccgcgcgcgctgccggaggaagcgccgccgggtccgctctgctctgggtccggctgggccatggagtccatgtctgagctcgcgccccgctgcctcttatttcctttgctgctgctgcttccgctgctgctccttcctgccccgaagctaggcccgagtcccgccggggctgaggagaccgactgggtgcgattgcccagcaaatgcgaagtgtgcaagtatgttgctgtggagctgaagtcggcttttgaggaaacgggaaagaccaaggaagtgattgacaccggctatggcatcctggacgggaagggctctggagtcaagtacaccaagtcggacttacggttaattgaagtcactgagaccatttgcaagaggcttctggactacagcctgcacaaggagaggactggcagcaaccggtttgccaagggtatgtcggagacctttgagacgctgcacaacctagtccacaaaggggtcaaggtggtgatggatatcccctatgagctgtggaacgagacctcagcagaggtggctgacctcaagaagcagtgtgacgtgctggtggaagagtttgaagaggtgattgaggactggtacaggaaccaccaggaggaagacctgactgaattcctctgtgccaaccacgtgctgaagggaaaggacacgagttgcctagcagagcggtggtctggcaagaagggggacatagcctccctgggagggaagaaatccaagaagaagcgcagcggagtcaagggctcctccagtggcagcagcaagcagaggaaggaactggggggcctgggggaggatgccaacgccgaggaggaggagggtgtgcagaaggcatcgcccctcccacacagcccccctgatgagctgtgagcccagcttagtgtccttgaatcaagacccctgacttcagagcttgggacacgcacacgcagcgcagcgcagctccagcaaggacagctgctgtccagcatcaggtctcctcccttggctgtgcccctttccttcccttgaacaacagcaagaggtggaaggatctggggtgctgggagacggcaccccaaagggaagaggaggaggagcagaaggcagctctctttctacacagtccccctcacgagctccggggtccacccagcatccccaggctgagatccaggctcctgacatggaagctgaagagcatgaggcacataagatgctcaccagcgcccccttcagccaggaaggactccgtgcagcctcagcagccaggcctgcctcttccttccaccaagcattctcttctgctggtccttgtcggatggtaaattcgagaacttccaggacaaactcgggtgtggcacaaaggggctggacgccagagccagagccacgccagagactgcagagagggcacctgacctaacccccctggaaagccaatctgcagttcccgtgtccacccactcctcctgaggacgcctcatgctctgcccagcccttctcccagggctaccagagtaaacaccttttggcctttcggtttggttcctgggtcctcatcagcctccagagtgtcccctcatcgatcttttttgcctttgtcccccatcccagggtgntggaaggccatcaccatcattgnaggnttaacctgtcagttactagaaggtgctgggagcgcccggggttggtttggggtaatcactnactggctctcagccttanaacactgcagccccttaatacagttccttctgttgtggtgactcccacgcccccacacacacaccataaaattatttggatgctgtttcataactgtaattttgctactgctatgaattgtaatgtaaatatttttggagatcgacagtaacg

SEQ ID NO: 17 - DNA, 200 bp, Artificial sequence
  Targeting vector
gccttggtcggtttcgcaagccgctagaggctaccgggcgaggggcgggccggagctcgccgttgccgtggttacccagagacacgtgcgcagtcccggaagcggccgggggaagctgctccgcgcgcgctgccggaggaagcgccgccgggtccgctctgctctgggtccggctgggccatggagtccatgtctgagct

SEQ ID NO: 18 - DNA, 200 bp, Artificial sequence
  Targeting vector
tgcgattgcccagcaaatgcgaaggtgagggggcggggccgcggggcgtagccaagcccgaggggcgggagggggcggggcctgtgggaagggtctgggcctggcaggacctgggctggggtctccttggccctgctgtgtgctttgcggcaatgctgggtgctgtgactctcggataacctggagatccctgcttttgg

SEQ ID NO: 19 - DNA, 5929 bp, Artificial sequence
  T243 specific construct
gcggccgcgagtcgacgaggccggccgattaattaaggctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggaggagtatttacggtaaactgcccacttggcagtacatcaagtgtatcatatgccaagtacgccccctattgacgtcaatgacggtaaatggcccgcctggcattatgcccagtacatgaccttacgggactttcctacttggcagtacatctacgtattagtcatcgctattaccatggttcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattggatccggcacgagggaggaagcgccgccgggtccgctctgctctgggtccggctgggccatggagtccatgtctgagctcgcgccccgctgcctcttatttcctttgctgctgctgcttccgctgctgctccttcctgccccgaagctaggcccgagtcccgccggggctgaggagaccgactgggtgcgattgcccagcaaatgcgaagtgtgcaagtatgttgctgtggagctgaagtcggcttttgaggaaacgggaaagaccaaggaagtgattgacaccggctatggcatcctggacgggaagggctctggagtcaagtacaccaagtcggacttacggttaattgaagtcactgagaccatttgcaagaggcttctggactacagcctgcacaaggagaggactggcagcaaccggtttgccaagggtatgtcggagacctttgagacgctgcacaacctagtccacaaaggggtcaaggtggtgatggatatcccctatgagctgtggaacgagacctcagcagaggtggctgacctcaagaagcagtgtgacgtgctggtggaagagtttgaagaggtgattgaggactggtacaggaaccaccaggaggaagacctgactgaattcctctgtgccaaccacgtgctgaagggaaaggacacgagttgcctagcagagcggtggtctggcaagaagggggacatagcctccctgggagggaagaaatccaagaagaagcgcagcggagtcaagggctcctccagtggcagcagcaagcagaggaaggaactggggggcctgggggaggatgccaacgccgaggaggaggagggtgtgcagaaggcatcgcccctcccacacagcccccctgatgagctgtgactcgaggaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagctcggggccaggtcggccgagcgatcgcgagaattcggcttaagtgagtcgtattacggactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcttcgcttggtaataaagcccgcttcggcgggcttttttttggttaactacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttctccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtaatgtgagttagctcactcattaggcaccccaggctttacactttatgcttccggctcgtatgttgtgtggaattgtgagcggataacaatttcacacaggaaacagctatgaccatgattacgccaagctacgtaatacgactcactag

(19) United States
(12) Patent Application Publication
(10) Pub. No.: US 2007/0174924 A1
(43) Pub. Date: Jul. 26, 2007

(54) Transgenic mice containing TRP gene disruptions

(76) Inventor: Keith D. Allen, Cary, NC (US)

Correspondence Address: JOHN E. BURKE;GREENBERG TRAURIG LLP, 1200 17TH STREET, SUITE 2400, DENVER, CO, 80202, US

(21) Appl. No.: 11/485,574

(22) Filed: Jul. 12, 2006

Related U.S. Application Data
(63) Continuation-in-part of application No. 09/696,686, filed on Oct. 26, 2000, now abandoned.
(60) Provisional application No. 60/161,488, filed on Oct. 26, 1999.

(51) Int. Cl. A01K 67/027

(52) U.S. Cl. 800/18

(57) ABSTRACT

The present disclosure relates to compositions and methods relating to the characterization of gene function. Specifically, the present disclosure provides transgenic mice comprising disruption in a trinucleotide repeat protein (TRP) gene. The present disclosure also provides methods of identifying agents that modulate TRP expression and function, useful models, and potential treatments for various disease states and disease conditions.

### RELATED APPLICATIONS

This application is a continuation-in-part of U.S. application Ser. No. 09/696,686, filed Oct. 26, 2000, which claims the benefit of U.S. Provisional Application No. 60/161,488, filed Oct. 26, 1999. The entire contents of each aforementioned provisional and nonprovisional application are incorporated herein by reference.

### FIELD OF THE INVENTION

The present disclosure relates to transgenic animals, compositions and methods relating to the characterization of gene function.

### BACKGROUND OF THE INVENTION

Many polymorphic trinucleotide repeats have been identified in the human genome. These mutations are produced by heritable, unstable DNA and are termed “dynamic mutations” because of changes in the number of repeat units inherited from generation to generation (Koshy, et al., Brain Pathol, 7:927-42 (1997)). Although these repeats are highly polymorphic, their number usually does not exceed 40 repeats in normal individuals (Online Mendelian Inheritance in Man, OMIM (TM). Johns Hopkins University, Baltimore, Md. MIM Number: 603279: jlewis: Jul. 14, 1999; World Wide Web URL: http://www.ncbi.nlm.nih.gov/omim; Koshy, et al. (1997)).

In contrast, abnormally expanded trinucleotide repeats have been found to cause disease (OMIM 603279). Expansions causing disease typically contain more than 40 trinucleotide repeats and tracts of 200 or more repeats have been reported (OMIM 603279; Slegtenhorst-Eegdeman, et al., Endocrinology, 139:156-62 (1998)). Four types of trinucleotide repeat expansions have been identified: (1) long cytosine-guanine-guanine (CGG) repeats in the two fragile X syndromes (FRAXA and FRAXE), (2) long cytosine-thymine-guanine (CTG) repeat expansions in myotonic dystrophy, (3) long guanine-adenine-adenine repeat expansions in Friedreich's ataxia and (4) short cytosine-adenine-guanine repeat expansions (CAG) which are implicated in neurodegenerative disorders. (Koshy, et al. (1997)).

At least 12 diseases, classified into Type 1 and Type 2 disorders, are caused by trinucleotide expansion mutation, most with neuropsychiatric features (Margolis, et al., Hum Genet., 100:114-122 (1997)). Type 1 disorders are caused by a (CAG)ₙ expansion in an open reading frame, resulting in an expanded glutamine repeat. Type 1 disorders include spinocerebellar ataxia type 1 (SCA1, Orr, et al., Nat Genet, 4:221-6 (1993); SCA2 (Imbert, et al., Nat Genet, 14:285-91 (1996); Pulst, et al., Nat Genet, 14:269-76 (1996); Sanpei, et al., Nat Genet, 14:277-84 (1996)); Machado-Joseph disease (MJD or SCA3, Kawaguchi, et al., Nat Genet, 8:221-8 (1994)); SCA6 (Zhuchenko, et al., Nat Genet, 15:62-9 (1997)); dentatutorubral pallidoluysioan atrophy (DRPLA, Koide, et al., Nat Genet, 6:9-13 (1994)); Huntington's disease (HD, Huntington's Disease Collaborative Research Group, Cell, 72:971-83 (1993)); and spinal and bulbar muscular atropy (SBMA, La Spada, Nature, 352:77-9 (1991)). Type 2 disorders can be caused by expansions in 5′ untranslated (Jacobsen's syndrome, Jones, et al., Nature, 376:145-9 (1995); fragile X syndrome, Fu, et al., Science, 1992 255:1256-8 (1992)), 3′ untranslated (myotonic dystrophy, Brook, et al., Cell, 68:799-808 (1992); Philips, et al., Science, 280:737-41 (1998)) and intronic regions (Fredreich's ataxia, Campuzano, et al., Science, 271:1423-7 (1996)). The mechanism and timing of the expansion events are poorly understood, however (Bates, et al., Hum Mol Genet., 6:1633-7 (1997)).

Diseases that are caused by trinucleotide repeat expansions exhibit a phenomenon called anticipation that cannot be explained by conventional Mendelian genetics (Koshy, et al. (1997)). Anticipation is defined as an increase in the severity of disease with an earlier age of onset of symptoms in successive generations. Anticipation is often influenced by the sex of the transmitting parent, and for most CAG repeat disorders, the disease is more severe when paternally transmitted. The severity and the age of onset of the disease have been correlated with the size of the repeats (Koshy, et al. (1997)). Longer expansions result in earlier onset and more severe clinical manifestations. The phenomenon of anticipation has led to the suspicion that instability in the expanded repeat underlies a given disorder (OMIM 603279).

The proteins harbouring expanded trinucleotide repeat tracts are unrelated and are widely expressed, with extensively overlapping expression patterns (Bates, et al. (1997)). Most are novel with the exception of the androgen receptor and the voltage gated alpha 1A calcium channel, which are mutated in spinal and bulbar muscular atrophy and spinocerebellar ataxia type 6. It is intriguing that CAG repeat proteins are ubiquitously expressed in both peripheral and central nervous tissue but in each neurological disorder only a select population of nerve cells are targeted for degeneration as a consequence of the expanded repeat (Koshy, et al. (1997)).

The mechanism by which expansion leads to neuronal dysfunuction and cell death is unknown (Bates, et al. (1997)). Current thinking is that the presence of a repeat tract confers a gain-of-function onto the involved gene, message or protein. For example, inappropriate interaction of the expanded CUG repeat region of myotonic dytrophy gene (MD) transcripts with CUG-binding proteins has been postulated to titrate-out proteins which normally comprise heterogeneous nuclear ribonucleoprotein particles (Bhagwati, et al., Biochim Biophys Acta, 1317:155-7 (1996); Philips, et al. (1998)). The creation of novel protein-protein interactions or aberrant protein folding, as well as alterations in flanking gene expression and chromatin structure have also been suggested as mechanisms by which trinucleotide expansion may cause disease (Thornton, et al., Nat. Genet., 16:407-9 (1997)).

Mouse models for trinucleotide repeat disorders hold great potential and promise for uncovering the molecular basis of these diseases and developing therapeutic interventions. Transgenic mice recapitulate many features of human disease and hence are excellent model systems to study the progression of disease in vivo. Using such mice, it will be possible to model both the pathogenic mechanism and the trinucleotide repeat instability in the mouse (Bates, et al. (1997)).

### SUMMARY OF THE INVENTION

The present disclosure generally relates to transgenic animals, as well as to compositions and methods relating to the characterization of gene function, and more specifically the present disclosure relates to genes encoding trinucleotide repeat proteins (TRP) such as gene T243.

The present disclosure provides a cell, preferably a stem cell and more preferably an embryonic stem (ES) cell, comprising a disruption in a target DNA sequence encoding a TRP. Preferably, the target DNA sequence is T243. In one embodiment, the stem cell is a murine ES cell. According to one embodiment, the disruption is produced by obtaining sequences homologous to the target DNA sequence and inserting the sequences into a targeting construct. The targeting construct is then introduced into the stem cell to produce a homologous recombinant which results in a disruption in the target DNA sequence.

In a more preferred embodiment, the targeting construct is generated using ligation-independent cloning to insert two different fragments of the homologous sequence into a vector having a second polynucleotide sequence, preferably a gene that encodes a positive selection marker such that the second polynucleotide sequence is positioned between the two different homologous sequence fragments in the construct. In one aspect of this embodiment, the homologous sequences may be obtained by: generating two primers complementary to the target; annealing the primers to complementary sequences in a mouse genomic DNA library containing the target region; and amplifying sequences homologous to the target region. The products of the amplification reaction, which have endpoints formed by the primers, are then isolated. Preferably, amplification is by PCR; more preferably, amplification is by long-range PCR. In another embodiment, the vector also includes a gene coding for a screening marker. In a further embodiment, the vector also includes recombinase sites flanking the positive selection marker.

The present disclosure further provides a vertebrate animal, preferably a mouse, having a disruption in a gene encoding a TRP. In one embodiment, the present disclosure provides a knockout mouse having a non-functional allele for the gene that naturally encodes and expresses a functional TRP. Included within the present disclosure is a knockout mouse having two non-functional alleles for the gene that naturally encodes and expresses functional TRP, and therefore is unable to produce wild type TRP. Preferably, the mouse is produced by injecting or otherwise introducing a stem cell comprising a disrupted gene encoding a TRP, either one described herein, or one available in the art, into a blastocyst. The resulting blastocyst is then injected into a pseudopregnant mouse which subsequently gives birth to a chimeric mouse containing the disrupted gene encoding the TRP in its germ line. A person skilled in the art will recognize that the chimeric mouse can be bred to generate mice with both heterozygous and homozygous disruptions in the gene encoding the TRP.

According to one embodiment, the disruption alters a TRP gene promoter, enhancer, or splice site such that the mouse does not express a functional TRP protein. In another embodiment, the disruption is an insertion, missense, frameshift or deletion mutation. The phenotype of such knockout mice can then be observed.

One aspect of the disclosure is a knockout mouse having a phenotype that includes reduced weight relative to an average normal, wild type adult mouse. Typically, the weight of the knockout mouse is reduced at least about 15%. Another aspect is a knockout mouse with a phenotype that includes decreased length relative to an average normal, wild type adult mouse. Commonly, length is decreased at least about 10%. Yet another aspect of the disclosure is a knockout mouse having a phenotype that includes a decreased ratio of weight to length relative to a normal, wild type adult mouse. Generally, a decrease of at least about 20% is observed.

In another embodiment of the disclosure, the knockout mouse has a phenotype including cartilage disease. Typically, abnormal cartilage is present and cartilage formation reduced.

Another aspect of the disclosure is a mouse having a phenotype that includes bone disease. Typically, the bone disease includes abnormal bone and reduced bone formation. In one embodiment, the phenotype of the knockout mouse is characterized by chondrodysplasia.

In yet another embodiment of the disclosure, the phenotype of the knockout mouse includes kidney disease. Commonly, kidney malformation is observed. In one embodiment, the phenotype of the knockout mouse includes renal dysplasia.

The present disclosure also provides a method of identifying agents capable of affecting a phenotype of a knockout mouse. According to this method, a putative agent is administered to a knockout mouse. The response of the knockout mouse to the putative agent is then measured and compared to the response of a “normal” or wild type mouse. The disclosure further provides agents identified according to such methods.

In a further embodiment of the disclosure, a knockout cell is provided in which a target DNA sequence encoding a TRP has been disrupted. According to one embodiment, the disruption inhibits production of wild type TRP. The cell or cell line can be derived from a knockout stem cell, tissue or animal. In a further embodiment, the cell is a stable cell culture.

The disclosure also provides cell lines comprising nucleic acid sequences encoding TRPs. Such cell lines may be capable of expressing such sequences by virtue of operable linkage to a promoter functional in the cell line. Preferably, expression of the sequence encoding the TRP is under the control of an inducible promoter.

In one aspect, the homozygous transgenic mouse exhibits, relative to a wild-type control mouse, at least one physical phenotypic abnormality selected from the group consisting of decreased body length, decreased body weight, decreased body weight to body length ratio, dry skin, decreased spleen weight, decreased spleen weight to body weight ratio, decreased liver weight, decreased kidney weight, decreased thymus weight, abnormal cartilage, reduction of bone formation, shortening of the axial skeleton, shortening of the appendicular skeleton, absence of growth plates in the sternebrae, discontinuous growth plates in the sternebrae, dysplastic changes in the kidney, decreased liver glycogen content, and juvenile lethality.

In another aspect, the homozygous transgenic mouse exhibits, relative to a wild-type control mouse, at least one behavioral phenotypic abnormality selected from the group consisting of hyperactivity, and increased total distance traveled in an open field test.

In a further aspect, the homozygous transgenic mouse exhibits, relative to a wild-type control mouse, a phenotypic abnormality comprising at least one change in associated gene expression selected from the group consisting of increased expression of leptin receptor precursor, increased expression of leptin receptor isoform A, increased expression of leptin receptor isoform F, decreased expression of glucose transporter 4 (Glut4) in skeletal muscle, increased expression of insulin-like growth factor (IGF) BP2, increased IGF BP I, and decreased expression of pre-pro-IGF.

In one aspect, the homozygous transgenic mouse exhibits, relative to a wild-type control mouse, at least one hematological phenotypic abnormality selected from the group consisting of increased white blood cells (WBC), increased neutrophils, and increased monocytes.

In another aspect, the homozygous transgenic mouse exhibits, relative to a wild-type control mouse, at least one serum chemistry phenotypic abnormality selected from the group consisting of increased creatinine, decreased calcium (Ca), decreased glucose, increased alkaline phosphatase (ALP), increased alanine aminotransferase (ALT), increased aspartate aminotransferase (AST), increased albumin, decreased globulin, increased total bilirubin (Bil T), increased cholesterol, and increased creatine kinase (CK).

In a further aspect, the transgenic mouse exhibits, relative to a wild-type control mouse, at least one densitometric phenotypic abnormality selected from the group consisting of decreased bone mineral density, decreased bone mineral content, decreased fat tissue mass, and decreased total tissue mass, when compared to wild-type control mice.

In one aspect, the homozygous transgenic mouse exhibits, relative to a wild-type control mouse, a metabolic phenotypic abnormality comprising decreased blood glucose levels in a glucose tolerance test.

In another aspect, the heterozygous transgenic mouse exhibits, relative to a wild-type control mouse, at least one phenotypic abnormality selected from the group consisting of decreased liver weight, increased blood creatinine, increased total distance traveled in the open field test, increased session time in the central zone in the open field test, and increased time immobile in the tail suspension test.

In a further aspect, the transgenic mouse overexpressing TRP exhibits, relative to a wild-type control mouse, at least one phenotypic abnormality selected from the group consisting of increased bone mineral density after estrogen depletion, increased blood glucose in a glucose tolerance test, hyperglycemia upon fasting, hyperglycemic state during an insulin secretion test, decrease in insulin levels following glucose challenge in a glucose-stimulated insulin secretion test, decreased body weights in a metabolic metrics study during a high fat diet. The present disclosure further provides novel, previously uncharacterized nucleic acid sequences encoding TRPs. Also provided is a method of identifying agents that interact with a TRP including the steps of contacting the TRP with an agent and detecting an agent/TRP complex.

The disclosure also provides methods for treating bone disease by administering to an appropriate subject an agent capable of affecting a phenotype of a knockout mouse to a subject. Appropriate subjects include, without limitation, mammals, including humans. In one embodiment, the bone disease is chondrodysplasia. The disclosure also provides methods for ameliorating the symptoms of bone disease, such as shortened bones, abnormal growth plates and reduced vertebrae. Among the agents which may be administered are T243 protein, a fragment thereof, as well as natural and synthetic analogs of T243.

Also provided are methods for treating cartilage disease by administering to a subject an agent capable of affecting a phenotype of a knockout mouse. In one embodiment, the cartilage disease is chondrodysplasia. Methods are also provided for ameliorating the symptoms of cartilage disease including large, irregular cartilage islands, short chondrocyte columns and thin irregular cartilage.

A method of treating kidney disease is also included within the scope of the disclosure. According to this method, an effective amount of an agent such as T243 protein, a T243 protein fragment, or a natural or synthetic analog of T243, is administered to a subject. In one embodiment, the kidney disease is renal dyplasia. The disclosure also includes methods for ameliorating symptoms associated with kidney disease such as small, abnormally formed kidneys.

The present disclosure also provides a method for determining whether expansion of the trinucleotide repeat in a TRP produces a phenotypic change. According to this method, a knockout stem cell in which a positive selection marker, flanked by recombinase sites, is contacted with a synthetic nucleic acid. The synthetic nucleic acid includes trinucleotide repeats flanked by recombinase target sites. In the presence of a recombinase which recognizes the recombinase target sites, recombination occurs between the recombinase sites in the synthetic nucleic acid and those flanking the positive selection marker by enzyme-assisted site-specific integration, thereby producing a transgenic stem cell. The phenotype of the resulting transgenic stem cell can then be compared with a normal, wild type stem cell, to determine whether trinucleotide expansion produces a phenotypic change. Preferably, the synthetic nucleic acid includes at least about 20 trinucleotide repeats. The enzyme-assisted site-specific integration can be, for example, a Cre recombinase-lox target system or an FLP recombinase-FRT target system.

The disclosure also provides a vertebrate, preferably a mouse, having a trinucleotide expansion of a gene encoding a TRP. In one embodiment, the mouse is produced by introducing a transgenic stem cell containing an expanded TRP gene into a blastocyst. The resulting blastocyst is then implanted into a pseudopregnant mouse which subsequently gives birth to a chimeric mouse containing the expanded trinucleotide repeat gene in its germ line. The chimeric mouse can then be bred to generate mice with either heterozygous or homozygous disruption in the gene encoding the TRP.

The present disclosure further provides novel, expanded TRP genes and the proteins encoded by these genes. Also provided is a method of identifying agents which interact with an expanded TRP including the steps of contacting the expanded TRP with an agent and detecting an agent/expanded TRP complex, thereby identifying agents which interact with the expanded TRP.

The disclosure also provides cell lines comprising nucleic acid sequences encoding expanded TRPs that are capable of expressing such sequences through operable linkage to promoters functional in the cell lines. Preferably, expression of the sequence encoding the expanded TRP is under the control of an inducible promoter.

In another embodiment, the phenotype (or phenotypic change) associated with a disruption in the TRP gene is used to predict the likely effects and side effects of a drug that antagonizes the TRP gene product. In this embodiment, the mouse is used to evaluate the gene as a “druggable target” i.e. to determine whether the development of drugs that target the TRP gene product would be a worthwhile focus for pharmaceutical research.

Definitions

As used herein, “gene targeting” is a type of homologous recombination that occurs when a fragment of genomic DNA is introduced into a mammalian cell and that fragment locates and recombines with endogenous homologous sequences.

“Disruption” of a target gene occurs when a fragment of genomic DNA locates and recombines with an endogenous homologous sequence such that production of the normal wild type gene product is inhibited. Non-limiting examples of disruption include insertion, missense, frameshift and deletion mutations. Gene targeting can also alter a promoter, enhancer, or splice site of a target gene to cause disruption, and can also involve replacement of a promoter with an exogenous promoter such as an inducible promoter described below.

As used herein, a “knockout mouse” is a mouse that contains within its genome a specific gene that has been disrupted or inactivated by the method of gene targeting. A knockout mouse includes both the heterozygote mouse (i.e., one defective allele and one wild-type allele) and the homozygous mutant (i.e., two defective alleles). Also included within the scope of the disclosure are hemizygous mice. It will be understood that certain genes, such as sex-linked genes in a male, are present in only one copy in the normal, wild type animal (i.e., are hemizygous in the normal wild type animal). A knockout mouse in which a gene which is normally hemizygous is disrupted will have a single defective allele of that gene.

The terms “polynucleotide” and “nucleic acid molecule” are used interchangeably to refer to polymeric forms of nucleotides of any length. The polynucleotides may contain deoxyribonucleotides, ribonucleotides and/or their analogs. Nucleotides may have any three-dimensional structure, and may perform any function, known or unknown. The term “polynucleotide” includes single-, double-stranded and triple helical molecules.

“Oligonucleotide” refers to polynucleotides of between 5 and about 100 nucleotides of single- or double-stranded DNA. Oligonucleotides are also known as oligomers or oligos and may be isolated from genes, or chemically synthesized by methods known in the art. A “primer” refers to an oligonucleotide, usually single-stranded, that provides a 3′-hydroxyl end for the initiation of enzyme-mediated nucleic acid synthesis.

The following are non-limiting embodiments of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A nucleic acid molecule may also comprise modified nucleic acid molecules, such as methylated nucleic acid molecules and nucleic acid molecule analogs. Analogs of purines and pyrimidines are known in the art, and include, but are not limited to, aziridinycytosine, 4-acetylcytosine, 5-fluorouracil, 5-bromouracil, 5-carboxymethylaminomethyl-2-thiouracil, 5-carboxymethyl-aminomethyluracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, pseudouracil, 5-pentylnyluracil and 2,6-diaminopurine. The use of uracil as a substitute for thymine in a deoxyribonucleic acid is also considered an analogous form of pyrimidine.

A “fragment” of a polynucleotide is a polynucleotide comprised of at least 9 contiguous nucleotides, preferably at least 15 contiguous nucleotides and more preferably at least 45 nucleotides, of coding or non-coding sequences.

As used herein, “base pair,” also designated “bp,” refers to the complementary nucleic acid molecules. In DNA there are four “types” of bases: the purine base adenine (A) is hydrogen bonded with the pyrimidine base thymine (T), and the purine base guanine (G) with the pyrimidine base cytosine (C). Each hydrogen bonded base pair set is also known as a Watson-Crick base-pair. A thousand base pairs is often called a kilobase pair, or kb. A “base pair mismatch” refers to a location in a nucleic acid molecule in which the bases are not complementary Watson-Crick pairs. The phrase “does not include at least one type of base at any position” refers to a nucleotide sequence which does not have one of the four bases at any position. For example, a sequence lacking one nucleotide (i.e., lacking one type of base) could be made up of A, G, T base pairs and contain no C residues.

As used herein, the term “construct” refers to an artificially assembled DNA segment to be transferred into a target tissue, cell line or animal, including human. Typically, the construct will include the gene or a sequence of particular interest, a marker gene and appropriate control sequences. The term “plasmid” refers to an autonomous, self-replicating extrachromosomal DNA molecule. In one embodiment, the plasmid construct of the present disclosure contains a positive selection marker positioned between two flanking regions of the gene of interest. Optionally, the construct can also contain a screening marker, for example, green fluorescent protein (GFP). If present, the screening marker is positioned outside of and some distance away from the flanking regions.

The term “polymerase chain reaction” or “PCR” refers to a method of amplifying a DNA base sequence using a heat-stable polymerase such as Taq polymerase, and two oligonucleotide primers; one complementary to the (+)-strand at one end of the sequence to be amplified and the other complementary to the (−)-strand at the other end. Because the newly synthesized DNA strands can subsequently serve as additional templates for the same primer sequences, successive rounds of primer annealing, strand elongation, and dissociation produce exponential and highly specific amplification of the desired sequence. PCR also can be used to detect the existence of the defined sequence in a DNA sample. “Long-range” refers to PCR conditions which allow amplification of large nucleotides stretches, for example, greater than 1 kb.

As used herein, the term “positive selection marker” refers to a gene encoding a product that enables only the cells that carry the gene to survive and/or grow under certain conditions. For example, plant and animal cells that express the introduced neomycin resistance (Neo^{r}) gene are resistant to the compound G418. Cells that do not carry the Neo^{r} gene marker are killed by G418. Other positive selection markers will be known to those of skill in the art.

“Positive-negative selection” refers to the process of selecting cells that carry a DNA insert integrated at a specific targeted location (positive selection) and also selecting against cells that carry a DNA insert integrated at a non-targeted chromosomal site (negative selection). Non-limiting examples of negative selection inserts include the gene encoding thymidine kinase (tk). Genes suitable for positive-negative selection are known in the art, see e.g., U.S. Pat. No. 5,464,764.

“Screening marker” or “reporter gene” refers to a gene that encodes a product that can readily be assayed. For example, reporter genes can be used to determine whether a particular DNA construct has been successfully introduced into a cell, organ or tissue. Non-limiting examples of screening markers include genes encoding for green fluorescent protein (GFP) or genes encoding for a modified fluorescent protein. “Negative screening marker” is not to be construed as negative selection marker; a negative selection marker typically kills cells that express it.

The term “vector” refers to a DNA molecule that can carry inserted DNA and be perpetuated in a host cell. Vectors are also known as cloning vectors, cloning vehicles or vehicles. The term includes vectors that function primarily for insertion of a nucleic acid molecule into a cell, replication vectors that function primarily for the replication of nucleic acid, and expression vectors that function for transcription and/or translation of the DNA or RNA. Also included are vectors that provide more than one of the above functions. In one embodiment, the vector contains sites useful in the methods described herein, for example, the vectors “pDG2” or “pDG4” as described herein.

A “host cell” includes an individual cell or cell culture which can be or has been a recipient for vector(s) or for incorporation of nucleic acid molecules and/or proteins. Host cells include progeny of a single host cell, and the progeny may not necessarily be completely identical (in morphology or in total DNA complement) to the original parent due to natural, accidental, or deliberate mutation. A host cell includes cells transfected with the constructs of the present disclosure.

The term “genomic library” refers to a collection of clones made from a set of randomly generated overlapping DNA fragments representing the genome of an organism. A “cDNA library” (complementary DNA library) is a collection of mRNA molecules present in a cell, tissue, or organism, turned into cDNA molecules with the enzyme reverse transcriptase, then inserted into vectors (other DNA molecules which can continue to replicate after addition of foreign DNA). Exemplary vectors for libraries include bacteriophage (also known as “phage”), which are viruses that infect bacteria, for example lambda phage. The library can then be probed for the specific cDNA (and thus mRNA) of interest. In one embodiment, library systems which combine the high efficiency of a phage vector system with the convenience of a plasmid system (for example, ZAP system from Stratagene, La Jolla, Calif.) are used in the practice of the present disclosure.

The term “homologous recombination” refers to the exchange of DNA fragments between two DNA molecules or chromatids at the site of homologous nucleotide sequences, i.e., those sequences preferably having at least about 70 percent sequence identity, typically at least about 85 percent identity, and preferably at least about 90 percent identity. Homology can be determined using a “BLASTN” algorithm. It is understood that homologous sequences can accommodate insertions, deletions and substitutions in the nucleotide sequence. Thus, linear sequences of nucleotides can be essentially identical even if some of the nucleotide residues do not precisely correspond or align.

As used herein the term “ligation-independent cloning” is used in the conventional sense to refer to incorporation of a DNA molecule into a vector or chromosome without the use of kinases or ligases. Ligation-independent cloning techniques are described, for instance, in Aslanidis & de Jong, Nucleic Acids Res., 18:6069-74 and U.S. patent application Ser. No. 07/847,298 (1991).

As used herein, the term “target sequence” (alternatively referred to as “target gene sequence” or “target DNA sequence”) refers to the nucleic acid molecule with any polynucleotide having a sequence in the general population that is not associated with any disease or discernible phenotype. It is noted that in the general population, wild-type genes may include multiple prevalent versions that contain alterations in sequence relative to each other and yet do not cause a discernible pathological effect. These variations are designated “polymorphisms” or “allelic variations.”

In one embodiment, the target DNA sequence comprises a portion of a particular gene or genetic locus in the individual's genomic DNA. Preferably, the target DNA sequence encodes a TRP, preferably having CTG trinucleotide repeats which encode leucine. According to one embodiment, the target DNA comprises part of a particular gene or genetic locus in which the function of the gene product is not known, for example, a gene identified using a partial cDNA sequence such as an EST. In one embodiment, the target TRP gene is T243, or any polynucleotide sequence homologous thereto, or orthologs thereof. Preferably, the target DNA sequence comprises SEQ ID NO: 1 (murine) or SEQ ID NO: 2 (human), or a naturally occurring allelic variation thereof.

The term “exonuclease” refers to an enzyme that cleaves nucleotides sequentially from the free ends of a linear nucleic acid substrate. Exonucleases can be specific for double or single-stranded nucleotides and/or directionally specific, for instance, 3′-5′ and/or 5′-3′. Some exonucleases exhibit other enzymatic activities, for example, T4 DNA polymerase is both a polymerase and an active 3′-5′ exonuclease. Other exemplary exonucleases include exonuclease III which removes nucleotides one at a time from the 5′-end of duplex DNA which does not have a phosphorylated 3′-end, exonuclease VI which makes oligonucleotides by cleaving nucleotides off of both ends of single-stranded DNA, and exonuclease lambda which removes nucleotides from the 5′ end of duplex DNA which have 5′-phosphate groups attached to them.

The term “recombinase” encompasses enzymes that induce, mediate or facilitate recombination, and other nucleic acid modifying enzymes that cause, mediate or facilitate the rearrangement of a nucleic acid sequence, or the excision or insertion of a first nucleic acid sequence from or into a second nucleic acid sequence. The “target site” of a recombinase is the nucleic acid sequence or region that is recognized (e.g., specifically binds to) and/or acted upon (excised, cut or induced to recombine) by the recombinase. As used herein, the expression “enzyme-directed site-specific recombination” is intended to include the following three events:

1. deletion of a pre-selected DNA segment flanked by recombinase target sites;

2. inversion of the nucleotide sequence of a pre-selected DNA segment flanked by recombinase target sites; and

3. reciprocal exchange of DNA segments proximate to recombinase target sites located on different DNA molecules.

### BRIEF DESCRIPTION OF THE DRAWING

FIG. 1 shows the nucleic acid sequence (SEQ ID NO: 1) encoding a murine TRP (SEQ ID NO: 3)(specifically, the expression product of T243); and the nucleic acid sequence (SEQ ID NO:2) encoding a human TRP (SEQ ID NO: 4).

FIG. 2 shows the amino acid sequence of a murine TRP (SEQ ID NO: 3) and the amino acid sequence of a human TRP (SEQ ID NO: 4).

FIG. 3 shows the nucleic acid sequences of oligonucleotide primers (SEQ ID NO: 5; SEQ ID NO: 6) used in PCR amplification of sequences homologous to target gene T243. Further shown are the same primers with cloning sites (SEQ ID NO: 7; SEQ ID NO:8); and nucleic acid sequences of primers (SEQ ID NO: 9; SEQ ID NO: 10) used to identify the aliquot of a library contained in target gene T243.

FIG. 4 shows the nucleic acid sequences of sequences homologous (SEQ ID NO: 11; SEQ ID NO: 12) to target gene T243 generated by PCR amplification.

FIG. 5 shows the nucleic acid sequence of the deleted gene fragment (SEQ ID NO: 13) of target gene T243 using a construct comprising homologous sequences (SEQ ID NO: 11; SEQ ID NO: 12). Further shown are the nucleic acid sequence of an expanded T243 gene (SEQ ID NO: 14) and the amino acid sequence of the corresponding expression product (SEQ ID NO: 15).

FIG. 6 shows the location and extent of the disrupted portion of a T243 gene (SEQ ID NO: 16), as well as the nucleotide sequences flanking the insert in the targeting construct.

FIG. 7 shows the sequences identified as SEQ ID NO: 17 and SEQ ID NO: 18, which were used as the 5′- and 3′-targeting arms (including the homologous sequences) in a T243 targeting construct, respectively.

FIG. 8A-C shows the nucleic acid sequence of a T243-specific construct used in production of transgenic mice by pronuclear injection (SEQ ID NO: 19).

FIG. 9 shows a Northern blot of two transgenic cell lines based upon Founder 7984 (CR-2), Founder 7985 (CR-7) and wild-type control (CR-6).

FIG. 10 shows a table of necropsy data for F2N0 homozygous (−/−), heterozygous (−/+) and wild-type (+/+) control mice (Table 3).

FIG. 11 shows a table of further necropsy data for F2N0 homozygous (−/−), heterozygous (−/+) and wild-type (+/+) control mice (Table 4).

FIG. 12 shows a table of hematology data for F2N0 homozygous (−/−), heterozygous (−/+) and wild-type (+/+) control mice (Table 5).

FIG. 13 shows a table of serum chemistry data for F2N0 homozygous (−/−), heterozygous (−/+), transgenic (TR) and wild-type (+/+) control mice (Table 6).

FIG. 14 shows a table of further serum chemistry data for F2N0 homozygous (−/−), heterozygous (−/+), transgenic (TR) and wild-type (+/+) control mice (Table 7).

FIG. 15 shows a table of densitometry data for homozygous (−/−), transgenic (TR) and wild-type (+/+) control mice (Table 8).

FIG. 16 shows bone mineral density (BMD) data for wild-type control (WT), high-expressing transgenic (H.E. TG), and low expressing transgenic (L.E. TG) mice following six weeks of estrogen depletion by ovariectomy.

FIG. 17 shows further bone mineral density data for homozygous mice and homozygous mice backcrossed to CD 1 (+/?) which survived to adulthood and exhibited about 20% increased bone mineral density when compared to homozygous mice (−/−).

FIG. 18 shows open field test data for F2N0 heterozygous and wild-type control mice (Table 9).

FIG. 19 shows open field test data for F2N0 homozygous (−/−), heterozygous (−/+) and wild-type (+/+) control mice at 17 days of age.

FIG. 20 shows a table of tail suspension test data for heterozygous (−/+) and wild-type control mice (+/+) (Table 10).

FIG. 21 shows data for mouse body weights at 54 days of age for mice homozygous and heterozygous for the T243 locus and + for the transgenic locus.

FIG. 22 shows Affymetrix GeneChip® data for expression of growth associated genes in homozygous (KO, n=3) and wild-type control mice (WT, n=3).

FIG. 23 shows Affymetrix GeneChip® data for expression of leptin receptor precursor genes in homozygous (KO, n=3) and wild-type control mice (WT, n=3).

FIG. 24 shows glucose transporter 4 mRNA expression data for homozygous and wild-type control mice by RT-PCR/TaqMan® Assay.

FIG. 25 shows liver glycogen content from homozygous (−/−), heterozygous (−/+), and wild-type control mice (+/+).

FIG. 26 shows a graph of glucose tolerance test data for male homozygous (−/−), heterozygous (−/+), and wild-type control mice (+/+).

FIG. 27 shows a graph of glucose tolerance test data for TRP (T2682) male and female wild-type and transgenic mice.

FIG. 28 shows blood glucose levels in male and female wild-type (WT) and transgenic (TG) mice.

FIG. 29 shows a graph of insulin suppression test (IST) data for wild-type (WT), high expressing transgenic (High TG) and low expressing transgenic (Low TG) mice.

FIG. 30 shows a graph of glucose stimulated insulin secretion test (GSIST) data for wild-type (WT) and high expressing transgenic (HE) mice.

FIG. 31 shows graphs of insulin and glucose levels in high expressing transgenic (H.E.), low expressing transgenic (L.E.) and wild-type (WT) control mice during the GSIST.

FIG. 32 shows a graph of body weights of male high expressing transgenic (TG high), low expressing transgenic (TG low) and wild-type (W/T) control mice mice during the high fat diet metabolic study.

### DETAILED DESCRIPTION OF THE INVENTION

The disclosure is based, in part, on the evaluation of the expression and role of genes and gene expression products, primarily those associated with trinucleotide repeat proteins. Among others, this permits the definition of disease pathways and the identification of targets in the pathway that are useful both diagnostically and therapeutically. For example, genes which are mutated or down-regulated under disease conditions may be involved in causing or exacerbating the disease condition. Treatments directed at up-regulating the activity of such genes or treatments which involve alternate pathways, may ameliorate the disease condition.

As used herein, “gene” refers to (a) a gene containing at least one of the DNA sequences disclosed herein; (b) any DNA sequence that encodes the amino acid sequence encoded by the DNA sequences disclosed herein and/or; (c) any DNA sequence that hybridizes to the complement of the coding sequences disclosed herein. Preferably, the term includes coding as well as noncoding regions, and preferably includes all sequences necessary for normal gene expression including promoters, enhancers and other regulatory sequences.

The disclosure also includes nucleic acid molecules, preferably DNA molecules, that hybridize to, and are therefore the complements of, the DNA sequences (a) through (c), in the preceding paragraph. Such in vitro hybridization conditions may be highly stringent or less highly stringent. Highly stringent conditions, for example, include hybridization to filter-bound DNA in 0.5M NaHPO₄, 7% sodium dodecyl sulfate (SDS), 1 mM EDTA at 65° C., and washing in 0.1×SSC/0.1% SDS at 68° C. (see Ausubel F. M., et al., eds., 1989, Current Protocols in Molecular Biology, Vol. I, Green Publishing Associates, Inc., and John Wiley & Sons, Inc., New York, at p. 2.10.3; Sambrook, Fritsch, and Maniatis, Molecular Cloning; A Laboratory Manual, Second Edition, Volume 2, Cold Springs Harbor Laboratory, Cold Springs, N.Y., pages 8.46-8.47 (1995), both of which are herein incorporated by reference) while less highly stringent conditions, such as moderately stringent conditions, e.g., washing in 0.2×SSC/0.1% SDS at 42° C. (Ausubel, et al., 1989, supra; Sambrook, et al., 1989, supra).

In instances wherein the nucleic acid molecules are deoxyoligonucleotides (“oligos”), highly stringent conditions may refer, e.g., to washing in 6×SSC/0.05% sodium pyrophosphate at 37° C. (for 14-base oligos), 48° C. (for 17-base oligos), 55° C. (for 20-base oligos), and 60° C. (for 23-base oligos). These nucleic acid molecules may act in vivo as target gene antisense molecules, useful, for example, in target gene regulation and/or as antisense primers in amplification reactions of target gene nucleic acid sequences. Further, such sequences may be used as part of ribozyme and/or triple helix sequences, also useful for target gene regulation. Still further, such molecules may be used as components of diagnostic methods whereby the presence of a disease-causing allele, may be detected.

The disclosure also encompasses (a) DNA vectors that contain any of the foregoing coding sequences and/or their complements (i.e., antisense); (b) DNA expression vectors that contain any of the foregoing coding sequences operatively associated with a regulatory element that directs the expression of the coding sequences; and (c) genetically engineered host cells that contain any of the foregoing coding sequences operatively associated with a regulatory element that directs the expression of the coding sequences in the host cell. As used herein, regulatory elements include but are not limited to inducible and non-inducible promoters, enhancers, operators and other elements known to those skilled in the art that drive and regulate expression. The disclosure includes fragments of any of the DNA sequences disclosed herein.

In addition to the gene sequences described above, homologues of such sequences, as may, for example be present in other species, may be identified and may be readily isolated, without undue experimentation, by molecular biological techniques well known in the art. Further, there may exist genes at other genetic loci within the genome that encode proteins which have extensive homology to one or more domains of such gene products. These genes may also be identified via similar techniques.

For example, the isolated differentially expressed gene sequence, or portion thereof, may be labeled and used to screen a cDNA library constructed from mRNA obtained from the organism of interest. Hybridization conditions will be of a lower stringency when the cDNA library was derived from an organism different from the type of organism from which the labeled sequence was derived. Alternatively, the labeled fragment may be used to screen a genomic library derived from the organism of interest, again, using appropriately stringent conditions. Such low stringency conditions will be well known to those of skill in the art, and will vary predictably depending on the specific organisms from which the library and the labeled sequences are derived. For guidance regarding such conditions see, for example, Sambrook, et al., 1989, Ausubel, et al., 1989.

In cases where the gene identified is the normal, or wild type, gene, this gene may be used to isolate mutant alleles of the gene. Such an isolation is preferable in processes and disorders which are known or suspected to have a genetic basis. Mutant alleles may be isolated from individuals either known or suspected to have a genotype which contributes to disease symptoms. Mutant alleles and mutant allele products may then be utilized in therapeutic and diagnostic assay systems.

A cDNA of the mutant gene may be isolated, for example, by using PCR, a technique which is well known to those of skill in the art. In this case, the first cDNA strand may be synthesized by hybridizing an oligo-dT oligonucleotide to mRNA isolated from tissue and known or suspected to be expressed in an individual putatively carrying the mutant allele, and by extending the new strand with reverse transcriptase. The second strand of the cDNA is then synthesized using an oligonucleotide that hybridizes specifically to the 5′ end of the normal gene. Using these two primers, the product is then amplified via PCR, cloned into a suitable vector, and subjected to DNA sequence analysis through methods well known to those of skill in the art. By comparing the DNA sequence of the mutant gene to that of the normal gene, the mutation(s) responsible for the loss or alteration of function of the mutant gene product can be ascertained.

Alternatively, a genomic or cDNA library can be constructed and screened using DNA or RNA, respectively, from a tissue known to or suspected of expressing the gene of interest in an individual suspected of or known to carry the mutant allele. The normal gene or any suitable fragment thereof may then be labeled and used as a probe to identify the corresponding mutant allele in the library. The clone containing this gene may then be purified through methods routinely practiced in the art, and subjected to sequence analysis.

Any technique known in the art may be used to introduce a target gene transgene into animals to produce the founder lines of transgenic animals. Such techniques include, but are not limited to pronuclear microinjection (U.S. Pat. No. 4,873,191); retrovirus mediated gene transfer into germ lines (Van der Putten, et al., Proc. Natl. Acad. Sci., USA, 82:6148-6152 (1985)); gene targeting in embryonic stem cells (Thompson, et al., Cell, 56:313-321 (1989)); electroporation of embryos (Lo, Mol Cell. Biol., 3:1803-1814 (1983)); and sperm-mediated gene transfer (Lavitrano, et al., Cell, 57:717-723 (1989)); etc. For a review of such techniques, see Gordon, Transgenic Animals, Intl. Rev. Cytol., 115:171-229 (1989), which is incorporated by reference herein in its entirety.

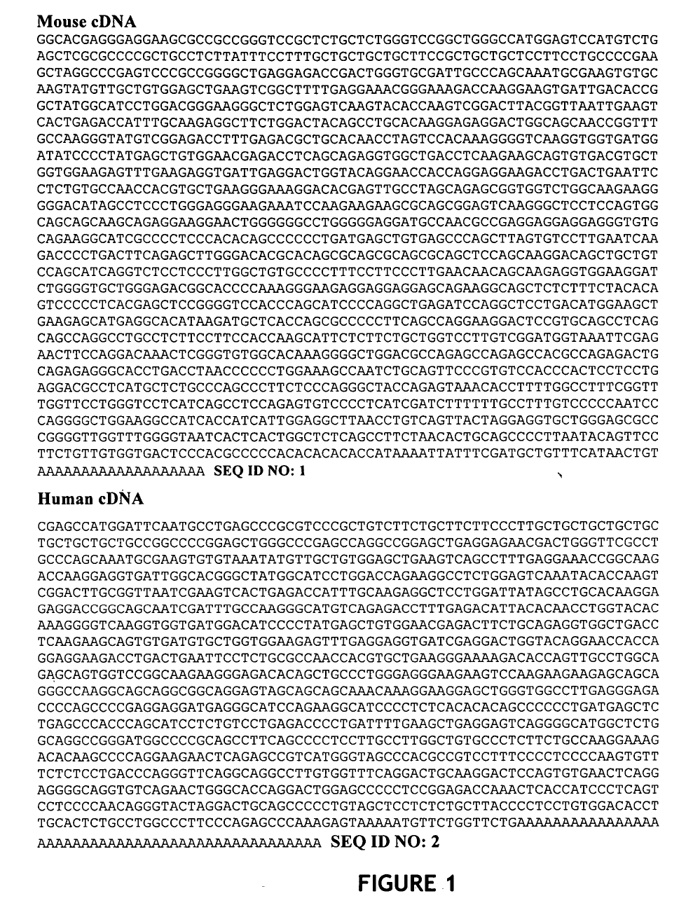

In one embodiment, homologous recombination is used to generate the knockout mice of the present disclosure. Preferably, the construct is generated in two steps by (1) amplifying (for example, using long-range PCR) sequences homologous to the target sequence, and (2) inserting another polynucleotide (for example a selectable marker) into the PCR product so that it is flanked by the homologous sequences. Typically, the vector is a plasmid from a plasmid genomic library. The completed construct is also typically a circular plasmid. Thus, as shown in FIG. 1, using long-range PCR with “outwardly pointing” oligonucleotides results in a vector into which a selectable marker can easily be inserted, preferably by ligation-independent cloning. The construct can then be introduced into ES cells, where it can disrupt the function of the homologous target sequence.

Homologous recombination may also be used to knockout genes in stem cells, and other cell types, which are not totipotent embryonic stem cells. By way of example, stem cells may be myeloid, lymphoid, or neural progenitor and precursor cells. Such knockout cells may be particularly useful in the study of target gene function in individual developmental pathways. Stem cells may be derived from any vertebrate species, such as mouse, rat, dog, cat, pig, rabbit, human, non-human primates and the like.

In cells which are not totipotent it may be desirable to knock out both copies of the target using methods which are known in the art. For example, cells comprising homologous recombination at a target locus which have been selected for expression of a positive selection marker (e.g., Neor) and screened for non-random integration, can be further selected for multiple copies of the selectable marker gene by exposure to elevated levels of the selective agent (e.g., G418). The cells are then analyzed for homozygosity at the target locus. Alternatively, a second construct can be generated with a different positive selection marker inserted between the two homologous sequences. The two constructs can be introduced into the cell either sequentially or simultaneously, followed by appropriate selection for each of the positive marker genes. The final cell is screened for homologous recombination of both alleles of the target.

In another aspect, two separate fragments of a clone of interest are amplified and inserted into a vector containing a positive selection marker using ligation-independent cloning techniques. In this embodiment, the clone of interest is generally from a phage library and is identified and isolated using PCR techniques. The ligation-independent cloning can be performed in two steps or in a single step.

According to one method, constructs are used having multiple sites where 5′-3′ single-stranded regions can be created. These constructs, preferably plasmids, include a vector capable of directional, four-way ligation-independent cloning.

The constructs typically include a sequence encoding a positive selection marker such as a gene encoding neomycin resistance; a restriction enzyme site on either side of the positive selection marker and a sequence flanking the restriction enzyme sites which does not contain one of the four base pairs. This configuration allows single-stranded ends to be created in the sequence by digesting the construct with the appropriate restriction enzyme and treating the fragments with a compound having exonuclease activity, for example T4 DNA polymerase.

In one preferred embodiment, a construct suitable for introducing targeted mutations into ES cells is prepared directly from a plasmid genomic library. Using long-range PCR with specific primers, a sequence of interest is identified and isolated from the plasmid library in a single step. Following isolation of this sequence, a second polynucleotide that will disrupt the target sequence can be readily inserted between two regions encoding the sequence of interest. Using this direct method a targeted construct can be created in as little as 72 hours. In another embodiment, a targeted construct is prepared after identification of a clone of interest in a phage genomic library as described in detail below.

The methods described herein obviate the need for hybridization isolation, restriction mapping and multiple cloning steps. Moreover, the function of any gene can be determined using these methods. For example, a short sequence (e.g., EST) can be used to design oligonucleotide probes. These probes can be used in the direct amplification procedure to create constructs or can be used to screen genomic or cDNA libraries for longer full-length genes. Thus, it is contemplated that any gene can be quickly and efficiently prepared for use in ES cells.

In one embodiment, constructs are prepared directly from a plasmid genomic library. The library can be produced by any method known in the art. Preferably, DNA from mouse ES cells is isolated and treated with a restriction endonuclease which cleaves the DNA into fragments. The DNA fragments are then inserted into a vector, for example a bacteriophage or phagemid (e.g., Lamda ZAP™, Stratagene, La Jolla, Calif.) systems. When the library is created in the ZAP™ system, the DNA fragments are preferably between about 5 and about 20 kilobases.

In one embodiment of the present disclosure, the targeting construct is prepared directly from a plasmid genomic library using the methods described in U.S. Pat. No. 6,815,185 issued Nov. 9, 2004, which is based on U.S. patent application Ser. No. 09/885,816, filed Jun. 19, 2001, which is a continuation of U.S. application Ser. No. 09/193,834, filed Nov. 17, 1998, now abandoned, which claims priority to provisional application No. 60/084,949, filed on May 11, 1998, and provisional application No. 60/084,194; and U.S. patent application Ser. No.: 08/971,310, filed Nov. 17, 1997, which was converted to provisional application No.: 60/084,194; the disclosure of which is incorporated herein in its entirety. Generally, a sequence of interest is identified and isolated from a plasmid library in a single step using, for example, long-range PCR. Following isolation of this sequence, a second polynucleotide that will disrupt the target sequence can be readily inserted between two regions encoding the sequence of interest. In accordance with this aspect, the construct is generated in two steps by (1) amplifying (for example, using long-range PCR) sequences homologous to the target sequence, and (2) inserting another polynucleotide (for example a selectable marker) into the PCR product so that it is flanked by the homologous sequences. Typically, the vector is a plasmid from a plasmid genomic library. The completed construct is also typically a circular plasmid.

In another embodiment, the targeting construct is designed in accordance with the regulated positive selection method described in U.S. patent application Ser. No. 09/954,483, filed Sep. 17, 2001, which is now published U.S. Patent Publication No. 20030032175, the disclosure of which is incorporated herein in its entirety. The targeting construct is designed to include a PGK-neo fusion gene having two lacO sites, positioned in the PGK promoter and an NLS-lacI gene comprising a lac repressor fused to sequences encoding the NLS from the SV40 T antigen. In another embodiment, the targeting construct may contain more than one selectable maker gene, including a negative selectable marker, such as the herpes simplex virus tk (HSV-tk) gene. The negative selectable marker may be operatively linked to a promoter and a polyadenylation signal.

Preferably, the organism(s) from which the libraries are made will have no discernible disease or phenotypic effects. Preferably, the library is a mouse library. This DNA may be obtained from any cell source or body fluid. Non-limiting examples of cell sources available in clinical practice include ES cells, liver, kidney, blood cells, buccal cells, cerviovaginal cells, epithelial cells from urine, fetal cells, or any cells present in tissue obtained by biopsy. Body fluids include urine, blood cerebrospinal fluid (CSF), and tissue exudates at the site of infection or inflammation. DNA extracted from the cells or body fluid using any method known in the art. Preferably, the DNA is extracted by adding 5 ml of lysis buffer (10 mM Tris-HCl pH 7.5), 10 mM EDTA (pH 8.0), 10 mM NaCl, 0.5% SDS and 1 mg/ml Proteinase K) to a confluent 100 mm plate of embryonic stem cells. The cells are then incubated at about 60° C. for several hours or until fully lysed. Genomic DNA is purified from the lysed cells by several rounds of gentle phenol:chloroform extraction followed by an ethanol precipitation. For convenience, the genomic library can be arrayed into pools.

In one embodiment, a sequence of interest is identified from the plasmid library using oligonucleotide primers and long-range PCR. Typically, the primers are outwardly-pointing primers which are designed based on sequence information obtained from a partial gene sequence, e.g., a cDNA or an EST sequence. As depicted for example in FIG. 1, the product will be a linear fragment that excludes the region which is located between each primer.

PCR conditions found to be suitable are described below in the Examples. It will be understood that optimal PCR conditions can be readily determined by those skilled in the art. (See, e.g., PCR 2: A Practical Approach (1995) eds. M. J. McPherson, B. D. Hames and G. R. Taylor, IRL Press, Oxford; Yu, et al., Methods Mol. Bio., 58:335-9 (1996); Munroe, et al., Proc. Natl Acad. Sci., USA, 92:2209-13 (1995)). PCR screening of libraries eliminates many of the problems and time-delay associated with conventional hybridization screening in which the library must be plated, filters made, radioactive probes prepared and hybridization conditions established. PCR screening requires only oligonucleotide primers to sequences (genes) of interest. PCR products can be purified by a variety of methods, including but not limited to, microfiltration, dialysis, gel electrophoresis and the like. It may be desirable to remove the polymerase used in PCR so that no new DNA synthesis can occur. Suitable thermostable DNA polymerases are commercially available, for example, Vent™ DNA Polymerase (New England Biolabs), Deep Vent™ DNA Polymerase (new England Biolabs), HotTub™ DNA Polymerase (Amersham), Thermo Sequenase™ (Amersham), rBst™ DNA Polymerase (Epicenter), Pfu™ DNA Polymerase (Stratagene), Amplitaq Gold™ (Perkin Elmer), and Expand™ (Boehringer-Mannheim).

To form the completed construct, a sequence which will disrupt the target sequence is inserted into the PCR-amplified product. For example, as described herein, the direct method involves joining the long-range PCR product (i.e., the vector) and one fragment (i.e., a gene encoding a selectable marker). As discussed above, the vector contains two different sequence regions homologous to the target DNA sequence. Preferably, the vector also contains a sequence encoding a selectable marker, such as ampicillin. The vector and fragment are designed so that, when treated to form single stranded ends, they will anneal such that the fragment is positioned between the two different regions of substantial homology to the target gene.

Although any method of cloning is suitable, it is preferred that ligation-independent cloning strategies be used to assemble the construct comprising two different homologous regions flanking a selectable marker. Ligation-independent cloning (LIC) is a strategy for the directional cloning of polynucleotides without the use of kinases or ligases. (See, e.g., Aslanidis et al., Nucleic Acids Res., 18:6069-74 (1990); Rashtchian, Current Opin. Biotech., 6:30-36 (1995)). Single-stranded tails (also referred to as cloning sites or annealing sequences) are created in LIC vectors, usually by treating the vector (at a digested restriction enzyme site) with T4 DNA polymerase in the presence of only one dNTP. The 3′ to 5′ exonuclease activity of T4 DNA polymerase removes nucleotides until it encounters a residue corresponding to the single dNTP present in the reaction mix. At this point, the 5′ to 3′ polymerase activity of the enzyme counteracts the exonuclease activity to prevent further excision. The vector is designed such that the single-stranded tails created are non-complementary. For example, in the pDG2 vector, none of the single-stranded tails of the four annealing sites are complementary to each other. PCR products are created by building appropriate 5′ extensions into oligonucleotide primers. The PCR product is purified to remove dNTPs (and original plasmid if it was used as template) and then treated with T4 DNA polymerase in the presence of the appropriate dNTP to generate the specific vector-compatible overhangs. Cloning occurs by annealing of the compatible tails. Single-stranded tails are created at the ends of the clone fragments, for example using chemical or enzymatic means. Complementary tails are created on the vector; however, to prevent annealing of the vector without insert, the vector tails are not complementary to each other. The length of the tails is at least about 5 nucleotides, preferably at least about 12 nucleotides, even more preferably at least about 20 nucleotides.

In one embodiment, placing the overlapping vector and fragment(s) in the same reaction is sufficient to anneal them. Alternatively, the complementary sequences are combined, heated and allowed to slowly cool. Preferably the heating step is between about 60° C. and about 100° C., more preferably between about 60° C. and 80° C., and even more preferably between 60° C. and 70° C. The heated reactions are then allowed to cool. Generally, cooling occurs rather slowly, for instance the reactions are generally at about room temperature after about an hour. The cooling must be sufficiently slow as to allow annealing. The annealed fragment/vector can be used immediately, or stored frozen at −20° C. until use.

Further, annealing can be performed by adjusting the salt and temperature to achieve suitable conditions. Hybridization reactions can be performed in solutions ranging from about 10 mM NaCl to about 600 mM NaCl, at temperatures ranging from about 37° C. to about 65° C. It will be understood that the stringency of the hybridization reaction is determined by both the salt concentration and the temperature. For instance, a hybridization performed in 10 mM salt at 37° C. may be of similar stringency to one performed in 500 mM salt at 65° C. For the present disclosure, any hybridization conditions may be used that form hybrids between homologous complementary sequences.

As shown in FIG. 1, in one embodiment, a construct is made after using any of these annealing procedure where the vector portion contains the two different regions of substantial homology to the target gene (amplified from the plasmid library using long-range PCR) and the fragment is a gene encoding a selectable marker.

After annealing, the construct is transformed into competent E. Coli cells, for example DH5-α cells by methods known in the art, to amplify the construct. The isolated construct is then ready for introduction into ES cells.

In another embodiment, a clone of interest is identified in a pooled genomic library using PCR. In one embodiment, the PCR conditions are such that a gene encoding a selectable marker can be inserted directly into the positively identified clone. The marker is positioned between two different sequences having substantial homology to the target DNA.

Genomic phage libraries can be prepared by any method known in the art and as described in the Examples. Preferably, a mouse embryonic stem cell library is prepared in lambda phage by cleaving genomic DNA into fragments of approximately 20 kilobases in length. The fragments are then inserted into any suitable lambda cloning vector, for example lambda Fix II or lambda Dash II (Stratagene, La Jolla, Calif.)

In order to quickly and efficiently screen a large number of clones from a library, pools may be created of plated libraries. In one embodiment, a genomic lambda phage library is plated at a density of approximately 1,000 clones (plaques) per plate. Sufficient plates are created to represent the entire genome of the organism several times over. For example, approximately 1 million clones (1000 plates) will yield approximately 8 genome equivalents. The plaques are then collected, for example by overlaying the plate with a buffer solution, incubating the plates and recollecting the buffer. The amount of buffer used will vary according to the plate size, generally one 100 mm diameter plate will be overlayed with approximately 4 ml of buffer and approximately 2 ml will be collected.

It will be understood that the individual plate lysates can be pooled at any time during this procedure and that they can be pooled in any combinations. For ease in later identification of single clones, however, it is preferable to keep each plate lysate separately and then make a pool. For example, each 2 ml lysate can be placed in a 96 well deep well plate. Pools can then be formed by taking an amount, preferably about 100 μl, from each well and combining them in the well of a new plate. Preferably, 100 μl of 12 individual plate lysates are combined in one well, forming a 1.2 ml pool representative of 12,000 clones of the library.

Each pool is then PCR-amplified using a set of PCR primers known to amplify the target gene. The target gene can be a known full-length gene or, more preferably, a partial cDNA sequence obtained from publicly available nucleic acid sequence databases such as GenBank or EMBL. These databases include partial cDNA sequences known as expressed sequence tags (ESTs). The oligonucleotide PCR primers can be isolated from any organism by any method known in the art or, preferably, synthesized by chemical means.

Once a positive clone of the target gene has been identified in a genomic library, two fragments encoding separate portions of the target gene must be generated. In other words, the flanking regions of the small known region of the target (e.g., EST) are generated. Although the size of each flanking region is not critical and can range from as few as 100 base pairs to as many as 100 kb, preferably each flanking fragment is greater than about 1 kb in length, more preferably between about 1 and about 10 kb, and even more preferably between about 1 and about 5 kb. One of skill in the art will recognize that although larger fragments may increase the number of homologous recombination events in ES cells, larger fragments will also be more difficult to clone.

In one embodiment, one of the oligonucleotide PCR primers used to amplify a flanking fragment is specific for the library cloning vector, for example lambda phage. Therefore, if the library is a lambda phage library, primers specific for the lambda phage arms can be used in conjunction with primers specific for the positive clone to generate long flanking fragments. Multiple PCR reactions can be set up to test different combinations of primers. Preferably, the primers used will generate flanking sequences between about 2 and about 6 kb in length.

Preferably, the oligonucleotide primers are designed with 5′ sequences complementary to the vector into which the fragments will be cloned. In addition, the primers are also designed so that the flanking fragments will be in the proper 3′-5′ orientation with respect to the vector and each other when the construct is assembled. Thus, using PCR-based methods, for example, positive clones can be identified by visualization of a band on an electrophoretic gel.

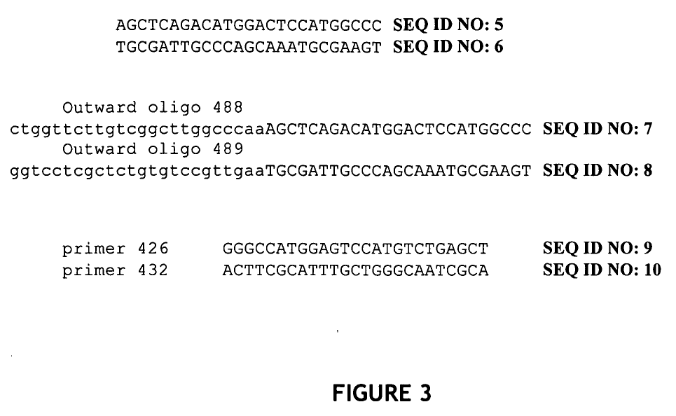

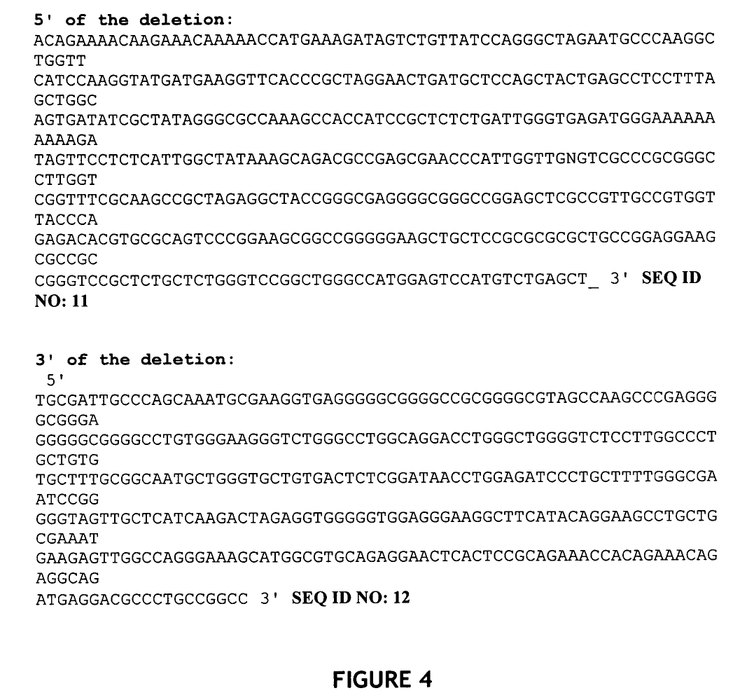

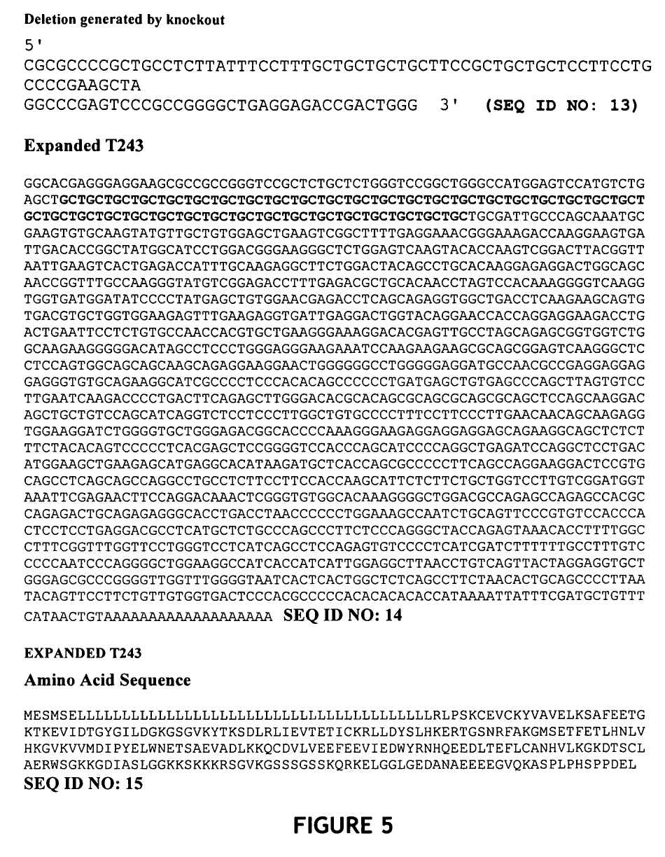

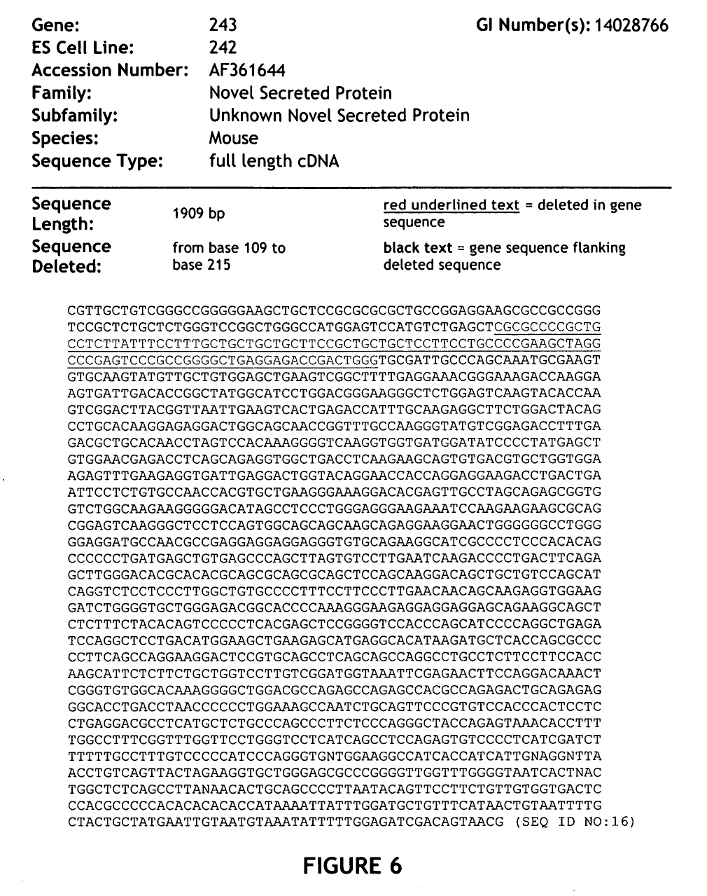

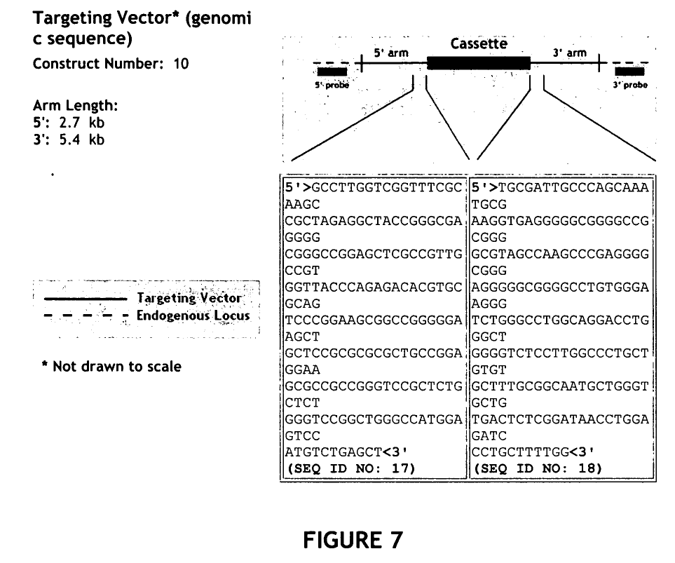

In one aspect, the cloning involves a vector and two fragments. The vector contains a positive selection marker, preferably Neo^{r}, and cloning sites on each side of the positive selection marker for two different regions of the target gene. Optionally, the vector also contains a sequence coding for a screening marker (reporter gene), preferably, positioned opposite the positive selection marker. The screening marker will be positioned outside the flanking regions of homologous sequences. FIG. 3A shows one embodiment of the vector with the screening marker, GFP, positioned on one side of the vector. However, the screening marker can be positioned anywhere between Not I and Site 4 on the side opposite the positive selection marker, Neo^{r}.

The specific nucleic acid ligation-independent cloning sites (also referred to herein as annealing sites) labeled “sites 1, 2, 3 or 4” in FIG. 1 are also shown herein. Generally, the cloning sites are lacking at least one type of base, i.e., thymine (T), guanine (G), cytosine (C) or adenine (A). Accordingly, reacting the vector with an enzyme that acts as both a polymerase and exonuclease in presence of only the one missing nucleotide will create an overhang. For example, T4 DNA polymerase acts as both a 3′-5′ exonuclease and a polymerase. Thus, when there are insufficient nucleotides available for the polymerase activity, T4 will act as an exonuclease. Specific overhangs can therefore be created by reacting the pDG2 vector with T4 DNA polymerase in the presence of dTTP only. Other enzymes useful in the practice of this disclosure will be known to those in the art, for instance uracil DNA glycosylase (UDG) (See, e.g., WO 93/18175). The vector exemplified herein has an overhand of 24 nucleotides. It will be known by those skilled in the art that as few as 5 nucleotides are required for successful ligation independent cloning.

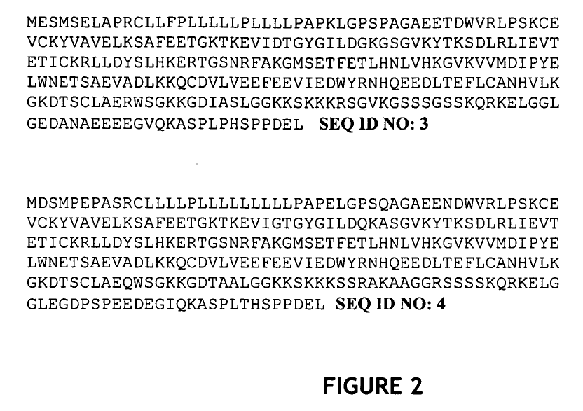

In another embodiment, a construct is assembled in a two-step cloning protocol. In the first step, each cloning region of homology is separately cloned into two of the annealing sites of the vector. For example, an “upstream” region of homology is cloned into annealing sites I and 2 while a separate cloning, a “downstream” region of homology is cloned into annealing sites 3 and 4. Once clones containing each single region of homology are identified, a targeting construct containing both regions of homology can be created by digesting each clone with restriction enzymes where one enzyme digests outside of annealing site 1 (e.g., Not I in FIG. 2A) and another enzyme digests between the positive selection marker and annealing site 3 (e.g., Sal I in FIG. 2A). The fragments containing the flanking homology regions from each construct will be purified (e.g., by gel electrophoresis) and combined using standard ligation techniques known in the art, to produce the resulting targeting construct.

In yet another embodiment, a construct according to one aspect of the present disclosure can be formed in a single-step, four-way ligation procedure. The vector and fragments are treated as described above. Briefly, the vector is treated to form two pieces, each piece having a single-stranded tail of specific sequence on each end. Likewise, the PCR-amplified flanking fragments are also treated to form single-stranded tails complementary to those of the vector pieces. The treated vector pieces and fragments are combined and allowed to anneal as described above. Because of the specificity of the single-stranded tails, the final construct will contain the fragments separated by the positive selection marker in the proper orientation.

The final plasmid constructs are amplified in bacteria, purified and can then be introduced into ES cells, or stored frozen at −20° C. until use. Where so desired, the vector is introduced into an embryonic stem cell line (e.g., by electroporation) and cells in which the introduced DNA has homologously recombined with the endogenous DNA are selected (see e.g., Li, et al., Cell, 69:91526 (1992)). The selected cells are then injected into a blastocyst (or other stage of development suitable for the purposes of creating a viable animal, such as, for example, a morula) of an animal (e.g., a mouse) to form chimeras (see e.g., Bradley, A. in Teratocarcinomas and Embryonic Stem Cells: A Practical Approach, E. J. Robertson, ed., IRL, Oxford, pp. 113-152 (1987)). Alternatively, selected ES cells can be allowed to aggregate with dissociated mouse embryo cells to form the aggregation chimera. A chimeric embryo can then be implanted into a suitable pseudopregnant female foster animal and the embryo brought to term. Chimeric progeny harbouring the homologously recombined DNA in their germ cells can be used to breed animals in which all cells of the animal contain the homologously recombined DNA. In one embodiment, chimeric progeny mice are used to generate a mouse with a heterozygous disruption in the target gene. Heterozygous knockout mice can then be mated. It is well know in the art that typically ¼ of the offspring of such matings will have a homozygous disruption in the target gene.

The heterozygous and homozygous knockout mice can then be compared to normal, wild type mice to determine whether disruption of the target gene causes phenotypic change, especially pathological change. In one embodiment, where the target DNA sequence is T243, the homozygous knockout mouse is reduced in weight relative to an average normal, wild type adult mouse. Weight is typically reduced by at least about 15%; more typically by about 30-90%; even more typically by about 40-80%; and most typically by about 60-70%.

In another embodiment, the length of homozygous knockout mouse is decreased relative to an average normal, wild type adult mouse. Length is generally decreased by at least about 10%; often by about 15-50%; more often by about 20-40%; and most often by about 25-35%.

The ratio of weight to length may also be decreased, relative to a normal, wild type adult mouse. Commonly, the ratio of weight to length is decreased at least about 20%, more commonly about 25-75%; even more commonly, about 30-65%; and most commonly about 40-55%.

Mice having a phenotype including both decreased length and reduced weight, are also observed. Such mice may also demonstrate a decreased ratio of weight to length.

In another embodiment of the disclosure, the knockout mouse has a phenotype including cartilage and/or bone disease. As used herein, “disease” refers to any alteration in the state of the body or of some of its organs, interrupting or disturbing the performance of the vital functions, and causing or threatening pain or weakness. Typically, in this embodiment, there is abnormal cartilage and a generalized reduction of bone formation.

Commonly observed pathological conditions include shortening of both the axial and appendicular skeleton. Proximal and distal bones of the limbs are proportionally shortened. Joint cartilage lacks alcian blue staining. Further aspects of this embodiment include thin growth plates of the distal femur and thin to absent epiphyseal cartilage. The disease may also present microfractures suggestive of growth plate fragility. Within the physes chondrocyte columns in the proliferating and hypertrophic zones are short in this embodiment. Cartilaginous spicules within the metaphysis are short and widely spaced; and occasional spicules are haphazardly oriented. Osteoblasts are abundant and frequently pile up along cartilaginous spicules. Epiphyseal cartilage is thin and often replaced by fibrous connective tissue. There is also decreased alcian blue staining of the epiphyseal surface. Cartilage at the epiphyseal/physeal junction is slightly flared with an irregular, prominent edge that overhangs the physis. Also included in this embodiment are irregular sternebrae; and growth plates are either lacking or are discontinuous. Large, irregular islands of cartilage extend into the shaft of the sternebra and occasionally have secondary ossification centers. Edges of the cartilage may also be flared. Another aspect includes variably ossified vertebral bodies which may be small and predominantly cartilaginous. Growth plates of these predominantly cartilaginous vertebrae are irregular and thin and the lateral processes are tapered. In one aspect of the disclosure, the disease is characterized as chondrodysplasia.

In yet another embodiment of the disclosure, the phenotype of the knockout mouse includes kidney disease. Typically, the kidneys are small and lack normal architecture. The cortex is thin and some glomeruli may be subcapsular. Subcapsular glomeruli are small with shrunken, hypercellular glomerular tufts. The corticomedullary area may lack radiating arcuate vessels and distinct tubule formation. Tubular epithelial cells within the corticomedullary junction are haphazardly arranged into sheets, piles and clusters. Some tubular epithelial cells are small and darkly basophilic indicating regeneration. Dysplastic changes are typically present in both kidneys and are most prominent in the corticomedullary junction and to a lesser extent in the cortex. According to one aspect of this disclosure, the kidney disease is characterized as renal dysplasia.

Other conditions of the pathological state may also be observed.

An additional feature that may be incorporated into the presently described vectors includes the use of recombinase target sites. Bacteriophage P1 Cre recombinase and flp recombinase from yeast plasmids are two non-limiting examples of site-specific DNA recombinase enzymes which cleave DNA at specific target sites (lox P sites for cre recombinase and frt sites for flp recombinase) and catalyze a ligation of this DNA to a second cleaved site. A large number of suitable alternative site-specific recombinases have been described, and their genes can be used in accordance with the method of the present disclosure. Such recombinases include the Int recombinase of bacteriophage λ (with or without Xis) (Weisberg, R. et. al., in Lambda II, (Hendrix, R., et al., Eds.), Cold Spring Harbor Press, Cold Spring Harbor, N.Y., pp. 211-50 (1983), herein incorporated by reference); TpnI and the β-lactamase transposons (Mercier, et al., J. Bacteriol., 172:3745-57 (1990)); the Tn3 resolvase (Flanagan & Fennewald J. Molec. Biol., 206:295-304 (1989); Stark, et al., Cell, 58:779-90 (1989)); the yeast recombinases (Matsuzaki, et al., J. Bacteriol., 172:610-18 (1990)); the B. subtilis SpoIVC recombinase (Sato, et al., J. Bacteriol. 172:1092-98 (1990)); the Flp recombinase (Schwartz & Sadowski, J. Molec.Biol., 205:647-658 (1989); Parsons, et al., J. Biol. Chem., 265:4527-33 (1990); Golic & Lindquist, Cell, 59:499-509 (1989); Amin, et al., J. Molec. Biol., 214:55-72 (1990)); the Hin recombinase (Glasgow, et al., J. Biol. Chem., 264:10072-82 (1989)); immunoglobulin recombinases (Malynn, et al., Cell, 54:453-460 (1988)); and the Cin recombinase (Haffter & Bickle, EMBO J., 7:3991-3996 (1988); Hubner, et al., J. Molec. Biol., 205:493-500 (1989)), all herein incorporated by reference. Such systems are discussed by Echols (J. Biol. Chem. 265:14697-14700 (1990)); de Villartay (Nature, 335:170-74 (1988)); Craig, (Ann. Rev. Genet., 22:77-105 (1988)); Poyart-Salmeron, et al., (EMBO J. 8:2425-33 (1989)); Hunger-Bertling, et al. (Mol Cell. Biochem., 92:107-16 (1990)); and Cregg & Madden (Mol. Gen. Genet., 219:320-23 (1989)), all herein incorporated by reference.

Cre has been purified to homogeneity, and its reaction with the loxP site has been extensively characterized (Abremski & Hess J. Mol. Biol. 259:1509-14 (1984), herein incorporated by reference). Cre protein has a molecular weight of 35,000 and can be obtained commercially from New England Nuclear/Du Pont. The cre gene (which encodes the Cre protein) has been cloned and expressed (Abremski, et al. Cell 32:1301-11 (1983), herein incorporated by reference). The Cre protein mediates recombination between two loxP sequences (Sternberg, et al. Cold Spring Harbor Symp. Quant. Biol. 45:297-309 (1981)), which may be present on the same or different DNA molecule. Because the internal spacer sequence of the loxP site is asymmetrical, two loxP sites can exhibit directionality relative to one another (Hoess & Abremski Proc. Natl. Acad. Sci. U.S.A. 81:1026-29 (1984)). Thus, when two sites on the same DNA molecule are in a directly repeated orientation, Cre will excise the DNA between the sites (Abremski, et al. Cell 32:1301-11 (1983)). However, if the sites are inverted with respect to each other, the DNA between them is not excised after recombination but is simply inverted. Thus, a circular DNA molecule having two loxP sites in direct orientation will recombine to produce two smaller circles, whereas circular molecules having two loxP sites in an inverted orientation simply invert the DNA sequences flanked by the loxP sites. In addition, recombinase action can result in reciprocal exchange of regions distal to the target site when targets are present on separate DNA molecules.

Recombinases have important application for characterizing gene function in knockout models. When the constructs described herein are used to disrupt target genes, a fusion transcript can be produced when insertion of the positive selection marker occurs downstream (3′) of the translation initiation site of the target gene. The fusion transcript could result in some level of protein expression with unknown consequence. It has been suggested that insertion of a positive selection marker gene can affect the expression of nearby genes. These effects may make it difficult to determine gene function after a knockout event since one could not discern whether a given phenotype is associated with the inactivation of a gene, or the transcription of nearby genes. Both potential problems are solved by exploiting recombinase activity. When the positive selection marker is flanked by recombinase sites in the same orientation, the addition of the corresponding recombinase will result in the removal of the positive selection marker. In this way, effects caused by the positive selection marker or expression of fusion transcripts are avoided.

Loss of function or null mutation models may be inadequate to characterize disease associated with TRP target genes. A number of published reports suggest that expansion of trinucleotide repeat regions in TRPs confer deleterious gains of function upon the resulting proteins. Such gains of function may involve novel or enhanced interaction with other proteins, increased resistance to proteolytic degradation, aberrant protein folding, and/or toxic accumulation of large, insoluble protein forms. It would therefore be of great value to mimic expansion of trinucleotide repeats in a TRP to determine whether expansion produces a phenotypic change that may be associated with a gain of function. Accordingly, one embodiment of the disclosure will involve the use of recombinases to bring about enzyme-assisted site-specific integration of a synthetic trinucleotide repeat at the site of disruption in a target gene. This embodiment will involve the reciprocal exchange ability of recombinase systems whereby a recombinase enzyme catalyzes the exchange of DNA distal to two target sites present on separate molecules. When the targeting construct used to generate a knockout stem cell includes a recombinase target site flanking the positive selection marker, recombination can occur between that site and a second site present on a synthetic nucleic acid in the presence of a recombinase enzyme.

One of skill in the art will recognize that the synthetic nucleic acid can be readily synthesized to include both the recombinase target site and repeated trinucleotides of any desired sequence. For example, the synthetic nucleic acid sequence can include repeats of CTG, encoding leucine, or CAG, encoding glutamine. Preferably, the synthetic nucleic acid will have at least about 20 trinucleotide repeats; more preferably, about at least about 40 trinucleotide repeats; most preferably, at least about 100 trinucleotide repeats.

The skilled artisan will also recognize the synthetic nucleic acid can be contacted with the disrupted gene by any standard laboratory methods for introducing DNA including, but not limited to, transfection, lipofection, or electroporation.

In one embodiment, purified recombinase enzyme is provided to the cell by direct microinjection. In another embodiment, recombinase is expressed from a co-transfected construct or vector in which the recombinase gene is operably linked to a functional promoter. An additional aspect of this embodiment is the use of tissue-specific or inducible recombinase constructs which allow the choice of when and where recombination occurs. One method for practicing the inducible forms of recombinase-mediated recombination involves the use of vectors that use inducible or tissue-specific promoters or other gene regulatory elements to express the desired recombinase activity. The inducible expression elements are preferably operatively positioned to allow the inducible control or activation of expression of the desired recombinase activity. Examples of such inducible promoters or other gene regulatory elements include, but are not limited to, tetracycline, metallothionine, ecdysone, and other steroid-responsive promoters, rapamycin responsive promoters, and the like (No, et al. Proc. Natl. Acad. Sci. USA, 93:3346-51 (1996); Furth, et al. Proc. NaCl. Acad Sci. USA, 91:9302-6 (1994)). Additional control elements that can be used include promoters requiring specific transcription factors such as viral, promoters. Vectors incorporating such promoters would only express recombinase activity in cells that express the necessary transcription factors.

The TRP gene sequences may also be used to produce TRP gene products. TRP gene products may include proteins that represent functionally equivalent gene products. Such an equivalent gene product may contain deletions, additions or substitutions of amino acid residues within the amino acid sequence encoded by the gene sequences described herein, but which result in a silent change, thus producing a functionally equivalent TRP gene product. Amino acid substitutions may be made on the basis of similarity in polarity, charge, solubility, hydrophobicity, hydrophilicity, and/or the amphipathic nature of the residues involved.

For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. “Functionally equivalent”, as utilized herein, refers to a protein capable of exhibiting a substantially similar in vivo activity as the endogenous gene products encoded by the TRP gene sequences. Alternatively, when utilized as part of an assay, “functionally equivalent” may refer to peptides capable of interacting with other cellular or extracellular molecules in a manner substantially similar to the way in which the corresponding portion of the endogenous gene product would.

Other TRP protein products useful according to the methods of the disclosure are peptides derived from or based on TRP produced by recombinant or synthetic means (TRP-derived peptides).

Mutant TRP proteins in which the trinucleotide regions are intentionally expanded, for example, by site-directed mutagensis, can also be produced. TRPs expanded by enzyme-assisted site-specific integration in stem cells can also be used.

The TRP and expanded TRP gene products may be produced by recombinant DNA technology using techniques well known in the art. Thus, methods for preparing the gene polypeptides and peptides of the disclosure by expressing nucleic acid encoding gene sequences are described herein. Methods which are well known to those skilled in the art can be used to construct expression vectors containing gene protein coding sequences and appropriate transcriptional/translational control signals. These methods include, for example, in vitro recombinant DNA techniques, synthetic techniques and in vivo recombination/genetic recombination (see, e.g., Sambrook, et al., 1989, supra, and Ausubel, et al., 1989, supra). Alternatively, RNA capable of encoding gene protein sequences may be chemically synthesized using, for example, automated synthesizers (see, e.g. Oligonucleotide Synthesis: A Practical Approach, Gait, M. J. ed., IRL Press, Oxford (1984)).

A variety of host-expression vector systems may be utilized to express the gene coding sequences of the disclosure. Such host-expression systems represent vehicles by which the coding sequences of interest may be produced and subsequently purified, but also represent cells which may, when transformed or transfected with the appropriate nucleotide coding sequences, exhibit the gene protein of the disclosure in situ. These include but are not limited to microorganisms such as bacteria (e.g., E. coli, B. subtilis) transformed with recombinant bacteriophage DNA, plasmid DNA or cosmid DNA expression vectors containing gene protein coding sequences; yeast (e.g. Saccharomyces, Pichia) transformed with recombinant yeast expression vectors containing the gene protein coding sequences; insect cell systems infected with recombinant virus expression vectors (e.g., baculovirus) containing the gene protein coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing gene protein coding sequences; or mammalian cell systems (e.g. COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5 K promoter).

In bacterial systems, a number of expression vectors may be advantageously selected depending upon the use intended for the gene protein being expressed. For example, when a large quantity of such a protein is to be produced, for the generation of antibodies or to screen peptide libraries, for example, vectors which direct the expression of high levels of fusion protein products that are readily purified may be desirable. Such vectors include, but are not limited, to the E. coli expression vector pUR278 (Ruther & Muller-Hill, EMBO J., 2:1791-94 (1983)), in which the gene protein coding sequence may be ligated individually into the vector in frame with the lac Z coding region so that a fusion protein is produced; pIN vectors (Inouye & Inouye, Nucleic Acids Res., 13:3101-09 (1985); Van Heeke & Schuster, J. Biol. Chem., 264:5503-9 (1989)); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene protein can be released from the GST moiety.

In one embodiment, full length cDNA sequences are appended with in-frame Bam HI sites at the amino terminus and Eco RI sites at the carboxyl terminus using standard PCR methodologies (Innis, et al. (eds) PCR Protocols: A Guide to Methods and Applications, Academic Press, San Diego (1990)) and ligated into the pGEX-2TK vector (Pharmacia, Uppsala, Sweden). The resulting cDNA construct contains a kinase recognition site at the amino terminus for radioactive labeling and glutathione S-transferase sequences at the carboxyl terminus for affinity purification (Nilsson, et al., EMBO J., 4: 1075-80 (1985); Zabeau and Stanley, EMBO J., 1: 1217-24 (1982)).

In an insect system, Autographa californica nuclear polyhedrosis virus (AcNPV) is used as a vector to express foreign genes. The virus grows in Spodoptera frugiperda cells. The gene coding sequence may be cloned individually into non-essential regions (for example the polyhedrin gene) of the virus and placed under control of an AcNPV promoter (for example the polyhedrin promoter). Successful insertion of gene coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed (see, e.g., Smith, et al., J. Virol. 46: 584-93 (1983); Smith, U.S. Pat. No. 4,745,051).

In mammalian host cells, a number of viral-based expression systems may be utilized. In cases where an adenovirus is used as an expression vector, the gene coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region El or E3) will result in a recombinant virus that is viable and capable of expressing gene protein in infected hosts. (e.g., see Logan & Shenk, Proc. Natl. Acad. Sci. USA, 81:3655-59 (1984)). Specific initiation signals may also be required for efficient translation of inserted gene coding sequences. These signals include the ATG initiation codon and adjacent sequences. In cases where an entire gene, including its own initiation codon and adjacent sequences, is inserted into the appropriate expression vector, no additional translational control signals may be needed. However, in cases where only a portion of the gene coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bitter, et al., Methods in Enzymol., 153:516-44 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, WI38, etc.

For long-term, high-yield production of recombinant proteins, stable expression is preferred. For example, cell lines which stably express the gene protein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells which stably integrate the plasmid into their chromosomes and grow, to form foci which in turn can be cloned and expanded into cell lines. This method may advantageously be used to engineer cell lines which express the gene protein. Such engineered cell lines may be particularly useful in screening and evaluation of compounds that affect the endogenous activity of the gene protein.

In one embodiment, control of timing and/or quantity of expression of the recombinant protein can be controlled using an inducible expression construct. Inducible constructs and systems for inducible expression of recombinant proteins will be well known to those skilled in the art. Examples of such inducible promoters or other gene regulatory elements include, but are not limited to, tetracycline, metallothionine, ecdysone, and other steroid-responsive promoters, rapamycin responsive promoters, and the like (No, et al., Proc. Natl. Acad. Sci. USA, 93:3346-51 (1996); Furth, et al., Proc. Natl. Acad. Sci. USA, 91:9302-6 (1994)). Additional control elements that can be used include promoters requiring specific transcription factors such as viral, particularly HIV, promoters. In one in embodiment, a Tet inducible gene expression system is utilized. (Gossen & Bujard, Proc. Natl. Acad. Sci. USA, 89:5547-51 (1992); Gossen, et al., Science, 268:1766-69 (1995)). Tet Expression Systems are based on two regulatory elements derived from the tetracycline-resistance operon of the E. coli Tn10 transposon-the tetracycline repressor protein (TetR) and the tetracycline operator sequence (tetO) to which TetR binds. Using such a system, expression of the recombinant protein is placed under the control of the tetO operator sequence and transfected or transformed into a host cell. In the presence of TetR, which is co-transfected into the host cell, expression of the recombinant protein is repressed due to binding of the TetR protein to the tetO regulatory element. High-level, regulated gene expression can then be induced in response to varying concentrations of tetracycline (Tc) or Tc derivatives such as doxycycline (Dox), which compete with tetO elements for binding to TetR. Constructs and materials for tet inducible gene expression are available commercially from CLONTECH Laboratories, Inc., Palo Alto, Calif.

When used as a component in an assay system, the gene protein may be labeled, either directly or indirectly, to facilitate detection of a complex formed between the gene protein and a test substance. Any of a variety of suitable labeling systems may be used including but not limited to radioisotopes such as ¹²⁵I; enzyme labeling systems that generate a detectable calorimetric signal or light when exposed to substrate; and fluorescent labels.

Where recombinant DNA technology is used to produce the gene protein for such assay systems, it may be advantageous to engineer fusion proteins that can facilitate labeling, immobilization and/or detection.

Indirect labeling involves the use of a protein, such as a labeled antibody, which specifically binds to either a gene product. Such antibodies include but are not limited to polyclonal, monoclonal, chimeric, single chain, Fab fragments and fragments produced by a Fab expression library.

Described herein are methods for the production of antibodies capable of specifically recognizing one or more gene epitopes. Such antibodies may include, but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above. Such antibodies may be used, for example, in the detection of a target TRP gene in a biological sample, or, alternatively, as a method for the inhibition of abnormal target gene activity. Thus, such antibodies may be utilized as part of disease treatment methods, and/or may be used as part of diagnostic techniques whereby patients may be tested for abnormal levels of target TRP gene proteins, or for the presence of abnormal forms of the such proteins.

For the production of antibodies to a gene, various host animals may be immunized by injection with a TRP protein, or a portion thereof. Such host animals may include but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as target gene product, or an antigenic functional derivative thereof. For the production of polyclonal antibodies, host animals such as those described above, may be immunized by injection with gene product supplemented with adjuvants as also described above.

Monoclonal antibodies, which are homogeneous populations of antibodies to a particular antigen, may be obtained by any technique which provides for the production of antibody molecules by continuous cell lines in culture. These include, but are not limited to the hybridoma technique of Kohler and Milstein, Nature, 256:495-7 (1975); and U.S. Pat. No. 4,376,110), the human B-cell hybridoma technique (Kosbor, et al., Immunology Today, 4:72 (1983); Cote, et al., Proc. Natl. Acad. Sci. USA, 80:2026-30 (1983)), and the EBV-hybridoma technique (Cole, et al., in Monoclonal Antibodies And Cancer Therapy, Alan R. Liss, Inc., New York, pp. 77-96 (1985)). Such antibodies may be of any immunoglobulin class including IgG, IgM, IgE, IgA, IgD and any subclass thereof. The hybridoma producing the mAb of this disclosure may be cultivated in vitro or in vivo. Production of high titers of mAbs in vivo makes this the presently preferred method of production.

In addition, techniques developed for the production of “chimeric antibodies” (Morrison, et al., Proc. Natl. Acad. Sci., 81:6851-6855 (1984); Takeda, et al., Nature, 314:452-54 (1985)) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. No. 4,946,778; Bird, Science 242:423-26 (1988); Huston, et al., Proc. Natl. Acad. Sci. USA, 85:5879-83 (1988); and Ward, et al., Nature, 334:544-46 (1989)) can be adapted to produce gene-single chain antibodies. Single chain antibodies are formed by linking the heavy and light chain fragments of the Fᵥ region via an amino acid bridge, resulting in a single chain polypeptide.

Antibody fragments which recognize specific epitopes may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse, et al., Science, 246:1275-81 (1989)) to allow rapid and easy identification of monoclonal Fab fragments with the desired specificity.

Described herein are cell- and animal-based systems which can be utilized as models for diseases. Animals of any species, including, but not limited to, mice, rats, rabbits, guinea pigs, pigs, micro-pigs, goats, and non-human primates, e.g., baboons, monkeys, and chimpanzees may be used to generate disease animal models. In addition, cells from humans may be used. These systems may be used in a variety of applications. For example, the cell- and animal-based model systems may be used to further characterize TRP genes. Such assays may be utilized as part of screening strategies designed to identify compounds which are capable of ameliorating disease symptoms. Thus, the animal- and cell-based models may be used to identify drugs, pharmaceuticals, therapies and interventions which may be effective in treating disease.

Cells that contain and express target gene sequences which encode TRPs, and, further, exhibit cellular phenotypes associated with disease, may be utilized to identify compounds that exhibit anti-disease activity.

Such cells may include non-recombinant monocyte cell lines, such as U937 (ATCC# CRL-1593), THP-1 (ATCC# TIB-202), and P388D1 (ATCC# TIB-63); endothelial cells such as HUVEC's and bovine aortic endothelial cells (BAEC's); as well as generic mammalian cell lines such as HeLa cells and COS cells, e.g., COS-7 (ATCC# CRL-1651). Further, such cells may include recombinant, transgenic cell lines. For example, the knockout mice of the disclosure may be used to generate cell lines, containing one or more cell types involved in a disease, that can be used as cell culture models for that disorder. While cells, tissues, and primary cultures derived from the disease transgenic animals of the disclosure may be utilized, the generation of continuous cell lines is preferred. For examples of techniques which may be used to derive a continuous cell line from the transgenic animals, see Small, et al., Mol. Cell Biol., 5:642-48 (1985).

Target gene sequences may be introduced into, and overexpressed in, the genome of the cell of interest, or, if endogenous target gene sequences are present, they may be either overexpressed or, alternatively disrupted in order to underexpress or inactivate target gene expression.

In order to overexpress a target gene sequence, the coding portion of the target gene sequence may be ligated to a regulatory sequence which is capable of driving gene expression in the cell type of interest. Such regulatory regions will be well known to those of skill in the art, and may be utilized in the absence of undue experimentation.

For underexpression of an endogenous target gene sequence, such a sequence may be isolated and engineered such that when reintroduced into the genome of the cell type of interest, the endogenous target gene alleles will be inactivated. Preferably, the engineered target gene sequence is introduced via gene targeting such that the endogenous target sequence is disrupted upon integration of the engineered target gene sequence into the cell's genome.

Cells transfected with target genes can be examined for phenotypes associated with a disease.

Compounds identified via assays may be useful, for example, in elaborating the biological function of the target gene product, and for ameliorating a disease. In instances whereby a disease condition results from an overall lower level of target gene expression and/or target gene product in a cell or tissue, compounds that interact with the target gene product may include compounds which accentuate or amplify the activity of the bound target gene protein. Such compounds would bring about an effective increase in the level of target gene product activity, thus ameliorating symptoms.

In vitro systems may be designed to identify compounds capable of binding a target TRP gene or an expanded TRP gene. Such compounds may include, but are not limited to, peptides made of D-and/or L-configuration amino acids (in, for example, the form of random peptide libraries; see e.g., Lam, et al., Nature, 354:82-4 (1991)), phosphopeptides (in, for example, the form of random or partially degenerate, directed phosphopeptide libraries; see, e.g., Songyang, et al., Cell, 72:767-78 (1993)), antibodies, and small organic or inorganic molecules. Compounds identified may be useful, for example, in modulating the activity of target gene proteins, preferably mutant target gene proteins, may be useful in elaborating the biological function of the target gene protein, may be utilized in screens for identifying compounds that disrupt normal target gene interactions, or may in themselves disrupt such interactions.

The principle of the assays used to identify compounds that bind to the target gene protein involves preparing a reaction mixture of the target gene protein or expanded target gene protein and the test compound under conditions and for a time sufficient to allow the two components to interact and bind, thus forming a complex which can be removed and/or detected in the reaction mixture. These assays can be conducted in a variety of ways. For example, one method to conduct such an assay would involve anchoring the target or expanded target gene protein or the test substance onto a solid phase and detecting target or expanded target gene protein/test substance complexes anchored on the solid phase at the end of the reaction. In one embodiment of such a method, the target gene protein may be anchored onto a solid surface, and the test compound, which is not anchored, may be labeled, either directly or indirectly.

In practice, microtitre plates are conveniently utilized. The anchored component may be immobilized by non-covalent or covalent attachments. Non-covalent attachment may be accomplished simply by coating the solid surface with a solution of the protein and drying. Alternatively, an immobilized antibody, preferably a monoclonal antibody, specific for the protein may be used to anchor the protein to the solid surface. The surfaces may be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the previously nonimmobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for target gene product or the test compound to anchor any complexes formed in solution, and a labeled antibody specific for the other component of the possible complex to detect anchored complexes.

Compounds that are shown to bind to a particular target gene product through one of the methods described above can be further tested for their ability to elicit a biochemical response from the target gene protein.

Cell-based systems may be used to identify compounds which may act to ameliorate a disease symptoms. For example, such cell systems may be exposed to a compound suspected of exhibiting an ability to ameliorate a disease symptoms, at a sufficient concentration and for a time sufficient to elicit such an amelioration of disease symptoms in the exposed cells. After exposure, the cells are examined to determine whether one or more of the disease cellular phenotypes has been altered to resemble a more normal or more wild type, non-disease phenotype.

In addition, animal-based disease systems, such as those described herein, may be used to identify compounds capable of ameliorating disease symptoms. Such animal models may be used as test substrates for the identification of drugs, pharmaceuticals, therapies, and interventions which may be effective in treating a disease or other phenotypic characteristic of the animal. For example, animal models may be exposed to a compound or agent suspected of exhibiting an ability to ameliorate disease symptoms, at a sufficient concentration and for a time sufficient to elicit such an amelioration of disease symptoms in the exposed animals. The response of the animals to the exposure may be monitored by assessing the reversal of disorders associated with the disease. Exposure may involve treating mother animals during gestation of the model animals described herein, thereby exposing embryos or fetuses to the compound or agent which may prevent or ameliorate the disease or phenotype. Neonatal, juvenile, and adult animals can also be exposed. Similar disease symptoms can arise from a variety of etiologies. Chondrodysplasias, for example, comprise a broad group of bone malformations that can result from defective collagen formation, disruption of signaling molecules [insulin-like growth factor (IGF), parathyroid hormone related protein (PTHrP), Indian hedgehog (Ihh), bone morphogenic proteins (BMPs)], or abnormal proteoglycans comprising the cartilage matrix (i.e. aggrecan). Primary bone diseases described in humans include osteogenesis imperfecta (defective type I collagen synthesis), mucopolysaccharidoses (lysosomal storage diseases that result in abnormal matrix), Blomstrand chondrodysplasia (defect of PTH/PTHrP hormone and/or receptor), multiple epiphyseal dysplasia (defective type IX collagen), and Schmid metaphyseal chondrodysplasia (defective type X collagen synthesis). Because of defective cartilage and/or cartilaginous matrix, there is reduced mineralization and bone formation. The term osteoporosis is used to denote a general reduction in bone mass and encompasses primary and secondary conditions. Primary osteoporotic conditions include idiopathic juvenile, idiopathic middle adulthood, postmenopausal, and senile osteoporosis. Secondary conditions that can result in osteoporosis include endocrine disorders (hyperparathyroidism, hyperthyroidism, hypothyroidism, hypogonadism, acromegaly, Cushing's disease, type 1 Diabetes, and Addison's disease), gastrointestinal disorders (malabsorption, vitamin C, D deficiency, malnutrition, and hepatic insufficiency), chronic obstructive pulmonary disease, Gaucher's disease, anemia, and homocystinuria. In addition to chondrocytes, osteoblasts play a critical role in bone formation. Osteoblasts have receptors for hormones (PTH, Vitamin D, estrogen), cytokines, and growth factors, and secrete collagenous and noncollagenous proteins. The noncollaginous proteins include cell adhesion proteins (osteopontin, fibronectin, thrombospondin), calcium binding proteins (osteonectin, bone sialoprotein), proteins involved in mineralization (osteocalcin), enzymes (collagenase and alkaline phosphatase), growth factors (IGF-1, TGF-B, PDGF) and cytokines (prostaglandins, IL-1, IL-6).

Furthermore, the aggregating proteoglycans of ground substance (aggrecan, versican, neurocan, and brevican) are important components of the extracellular matrix. The recently described ligand for aggrecan and versican, fibulin-1 (Aspberg, et al., J. Biol Chem, 274:20444-9 (1999)), is strongly expressed in developing cartilage and bone.

Another group of symptoms, renal dysplasias and hypoplasias, account for 20% of chronic renal failure in children (Cotran, et al., Robbins Pathologic Basis of Disease, Saunders, Pa. (1994)). Congenital renal disease can be hereditary but is most often the result of an acquired developmental defect that arises during gestation. In affected individuals, urogenital differentiation is evident by 8.5 to 9 days of gestation in the mouse (corresponding to gestational days 22-24 in humans). During development, dysplasias have been hypothesized to result from abnormal cell differentiation, leading to sustained cellular proliferation and transepithelial fluid secretion that may result in cyst formation (Grantham, et al. (1993) Adv Intern Med 38:409-20), or an extracellular matrix defect that, in turn, affects epithelial differentiation (Calvet, et al., J Histochem Cytochem, 41:1223-31 (1993)). Growth factors that are common to bone and renal development include Insulin-like growth factor and BMPs. However, chronic renal failure can also affect bone formation because of calcium/phosphorus and acid/base imbalances.

One of skill in the art will recognize that a given agent may be effective in ameliorating similar symptoms caused by disparate etiologies. Thus, a given agent may be useful in the treatment of a variety of diseases.

Among the agents which may exhibit the ability to ameliorate disease symptoms are antisense, ribozyme, and triple helix molecules. Such molecules may be designed to reduce or inhibit mutant target gene activity. Techniques for the production and use of such molecules are well known to those of skill in the art.

Anti-sense RNA and DNA molecules act to directly block the translation of mRNA by hybridizing to targeted mRNA and preventing protein translation. With respect to antisense DNA, oligodeoxyribonucleotides derived from the translation initiation site, e.g., between the −10 and +10 regions of the target gene nucleotide sequence of interest, are preferred.

Ribozymes are enzymatic RNA molecules capable of catalyzing the specific cleavage of RNA. The mechanism of ribozyme action involves sequence-specific hybridization of the ribozyme molecule to complementary target RNA, followed by an endonucleolytic cleavage. The composition of ribozyme molecules must include one or more sequences complementary to the target gene mRNA, and must include the well known catalytic sequence responsible for mRNA cleavage. For this sequence, see U.S. Pat. No. 5,093,246, which is incorporated by reference herein in its entirety. As such within the scope of the disclosure are engineered hammerhead motif ribozyme molecules that specifically and efficiently catalyze endonucleolytic cleavage of RNA sequences encoding target gene proteins.

Specific ribozyme cleavage sites within any potential RNA target are initially identified by scanning the molecule of interest for ribozyme cleavage sites which include the following sequences, GUA, GUU and GUC. Once identified, short RNA sequences of between 15 and 20 ribonucleotides corresponding to the region of the target gene containing the cleavage site may be evaluated for predicted structural features, such as secondary structure, that may render the oligonucleotide sequence unsuitable. The suitability of candidate sequences may also be evaluated by testing their accessibility to hybridization with complementary oligonucleotides, using ribonuclease protection assays.

Nucleic acid molecules to be used in triple helix formation for the inhibition of transcription should be single stranded and composed of deoxyribonucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

It is possible that the antisense, ribozyme, and/or triple helix molecules described herein may reduce or inhibit the transcription (triple helix) and/or translation (antisense, ribozyme) of mRNA produced by both normal and mutant target gene alleles. In order to ensure that substantially normal levels of target gene activity are maintained, nucleic acid molecules that encode and express target gene polypeptides exhibiting normal activity may be introduced into cells that do not contain sequences susceptible to whatever antisense, ribozyme, or triple helix treatments are being utilized. Alternatively, it may be preferable to coadminister normal target gene protein into the cell or tissue in order to maintain the requisite level of cellular or tissue target gene activity.

Anti-sense RNA and DNA, ribozyme, and triple helix molecules of the disclosure may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various well-known modifications to the DNA molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

Antibodies that are both specific for target gene protein and interfere with its activity may be used to inhibit target gene function. Antibodies that are specific for expanded target gene protein and interfere with the unique interactions of that protein, especially functions attributable novel gains of function associated with trinucleotide expansion, may also be used to inhibit expanded target gene function. Of particular interest are antibodies directed to expanded trinucleotide regions of TRPs. Such antibodies may be generated using standard techniques against the proteins themselves or against peptides corresponding to portions of the proteins. Such antibodies include but are not limited to polyclonal, monoclonal, Fab fragments, single chain antibodies, chimeric antibodies, etc.

In instances where the target gene protein is intracellular and whole antibodies are used, internalizing antibodies may be preferred. However, lipofectin liposomes may be used to deliver the antibody or a fragment of the Fab region which binds to the target gene epitope into cells. Where fragments of the antibody are used, the smallest inhibitory fragment which binds to the target or expanded target protein's binding domain is preferred. For example, peptides having an amino acid sequence corresponding to the domain of the variable region of the antibody that binds to the target gene protein may be used. Such peptides may be synthesized chemically or produced via recombinant DNA technology using methods well known in the art (see, e.g., Creighton, Proteins : Structures and Molecular Principles (1984) W.H. Freeman, New York 1983, supra; and Sambrook, et al., 1989, supra). Alternatively, single chain neutralizing antibodies which bind to intracellular target gene epitopes may also be administered. Such single chain antibodies may be administered, for example, by expressing nucleotide sequences encoding single-chain antibodies within the target cell population by utilizing, for example, techniques such as those described in Marasco, et al., Proc. Natl. Acad. Sci. USA, 90:7889-93 (1993).

Antibodies that are specific for one or more extracellular domains of the TRP or expanded TRP and that interfere with its activity, are particularly useful in treating disease. Such antibodies are especially efficient because they can access the target domains directly from the bloodstream. Any of the administration techniques described below which are appropriate for peptide administration may be utilized to effectively administer inhibitory target gene antibodies to their site of action.

RNA sequences encoding target gene protein may be directly administered to a patient exhibiting disease symptoms, at a concentration sufficient to produce a level of target gene protein such that disease symptoms are ameliorated.

Patients may be treated by gene replacement therapy. One or more copies of a normal target gene, or a portion of the gene that directs the production of a normal target gene protein with target gene function, may be inserted into cells using vectors which include, but are not limited to adenovirus, adeno-associated virus, and retrovirus vectors, in addition to other particles that introduce DNA into cells, such as liposomes. Additionally, techniques such as those described above may be utilized for the introduction of normal target gene sequences into human cells.

Cells, preferably, autologous cells, containing normal target gene expressing gene sequences may then be introduced or reintroduced into the patient at positions which allow for the amelioration of disease symptoms.

The identified compounds that inhibit target or expanded target gene expression, synthesis and/or activity can be administered to a patient at therapeutically effective doses to treat or ameliorate the disease. A therapeutically effective dose refers to that amount of the compound sufficient to result in amelioration of symptoms of the disease.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the disclosure, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Pharmaceutical compositions for use in accordance with the present disclosure may be formulated in conventional manner using one or more physiologically acceptable carriers or excipients. Thus, the compounds and their physiologically acceptable salts and solvates may be formulated for administration by inhalation or insufflation (either through the mouth or the nose) or oral, buccal, parenteral, topical, subcutaneous, intraperitoneal, intraveneous, intrapleural, intraoccular, intraarterial, or rectal administration. It is also contemplated that pharmaceutical compositions may be administered with other products that potentiate the activity of the compound and optionally, may include other therapeutic ingredients.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present disclosure are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides. Oral ingestion is possibly the easiest method of taking any medication. Such a route of administration, is generally simple and straightforward and is frequently the least inconvenient or unpleasant route of administration from the patient's point of view. However, this involves passing the material through the stomach, which is a hostile environment for many materials, including proteins and other biologically active compositions. As the acidic, hydrolytic and proteolytic environment of the stomach has evolved efficiently to digest proteinaceous materials into amino acids and oligopeptides for subsequent anabolism, it is hardly surprising that very little or any of a wide variety of biologically active proteinaceous material, if simply taken orally, would survive its passage through the stomach to be taken up by the body in the small intestine. The result, is that many proteinaceous medicaments must be taken in through another method, such as parenterally, often by subcutaneous, intramuscular or intravenous injection.

Pharmaceutical compositions may also include various buffers (e.g., Tris, acetate, phosphate), solubilizers (e.g., Tween, Polysorbate), carriers such as human serum albumin, preservatives (thimerosol, benzyl alcohol) and anti-oxidants such as ascorbic acid in order to stabilize pharmacetical activity. The stabilizing agent may be a detergent, such as tween-20, tween-80, NP-40 or Triton X-100. EBP may also be incorporated into particulate preparations of polymeric compounds for controlled delivery to a patient over an extended period of time. A more extensive survey of components in pharmaceutical compositions is found in Remington's Pharmaceutical Sciences, 18th ed., A. R. Gennaro, ed., Mack Publishing, Easton, Pa. (1990).

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

A variety of methods may be employed to diagnose disease conditions associated with a TRP. Specifically, reagents may be used, for example, for the detection of the presence of target gene mutations, or the detection of either over or under expression of target gene mRNA.

The methods described herein may be performed, for example, by utilizing pre-packaged diagnostic kits comprising at least one specific gene nucleic acid or anti-gene antibody reagent described herein, which may be conveniently used, e.g., in clinical settings, to diagnose patients exhibiting disease symptoms or at risk for developing disease.

Any cell type or tissue, preferably monocytes, endothelial cells, or smooth muscle cells, in which the gene is expressed may be utilized in the diagnostics described below.

DNA or RNA from the cell type or tissue to be analyzed may easily be isolated using procedures which are well known to those in the art. Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, PCR In Situ Hybridization: Protocols and Applications, Raven Press, N.Y. (1992)).

Gene nucleotide sequences, either RNA or DNA, may, for example, be used in hybridization or amplification assays of biological samples to detect disease-related gene structures and expression. Such assays may include, but are not limited to, Southern or Northern analyses, restriction fragment length polymorphism assays, single stranded conformational polymorphism analyses, in situ hybridization assays, and polymerase chain reaction analyses. Such analyses may reveal both quantitative aspects of the expression pattern of the gene, and qualitative aspects of the gene expression and/or gene composition. That is, such aspects may include, for example, point mutations, insertions, deletions, chromosomal rearrangements, and/or activation or inactivation of gene expression.

Preferred diagnostic methods for the detection of gene-specific nucleic acid molecules may involve for example, contacting and incubating nucleic acids, derived from the cell type or tissue being analyzed, with one or more labeled nucleic acid reagents under conditions favorable for the specific annealing of these reagents to their complementary sequences within the nucleic acid molecule of interest. Preferably, the lengths of these nucleic acid reagents are at least 9 to 30 nucleotides. After incubation, all non-annealed nucleic acids are removed from the nucleic acid:fingerprint molecule hybrid. The presence of nucleic acids from the fingerprint tissue which have hybridized, if any such molecules exist, is then detected. Using such a detection scheme, the nucleic acid from the tissue or cell type of interest may be immobilized, for example, to a solid support such as a membrane, or a plastic surface such as that on a microtitre plate or polystyrene beads. In this case, after incubation, non-annealed, labeled nucleic acid reagents are easily removed. Detection of the remaining, annealed, labeled nucleic acid reagents is accomplished using standard techniques well-known to those in the art.

Alternative diagnostic methods for the detection of gene-specific nucleic acid molecules may involve their amplification, e.g., by PCR (the experimental embodiment set forth in Mullis U.S. Pat. No. 4,683,202 (1987)), ligase chain reaction (Barany, Proc. Natl. Acad. Sci. USA, 88:189-93 (1991)), self sustained sequence replication (Guatelli, et al., Proc. Natl. Acad. Sci. USA, 87:1874-78 (1990)), transcriptional amplification system (Kwoh, et al., Proc. Natl. Acad. Sci. USA, 86:1173-77 (1989)), Q-Beta Replicase (Lizardi, P. M., et al., Bio/Technology, 6:1197 (1988)), or any other nucleic acid amplification method, followed by the detection of the amplified molecules using techniques well known to those of skill in the art. These detection schemes are especially useful for the detection of nucleic acid molecules if such molecules are present in very low numbers.

In one embodiment of such a detection scheme, a cDNA molecule is obtained from an RNA molecule of interest (e.g., by reverse transcription of the RNA molecule into cDNA). Cell types or tissues from which such RNA may be isolated include any tissue in which wild type fingerprint gene is known to be expressed, including, but not limited, to monocytes, endothelium, and/or smooth muscle. A sequence within the cDNA is then used as the template for a nucleic acid amplification reaction, such as a PCR amplification reaction, or the like. The nucleic acid reagents used as synthesis initiation reagents (e.g., primers) in the reverse transcription and nucleic acid amplification steps of this method may be chosen from among the gene nucleic acid reagents described herein. The preferred lengths of such nucleic acid reagents are at least 15-30 nucleotides. For detection of the amplified product, the nucleic acid amplification may be performed using radioactively or non-radioactively labeled nucleotides. Alternatively, enough amplified product may be made such that the product may be visualized by standard ethidium bromide staining or by utilizing any other suitable nucleic acid staining method.

Antibodies directed against wild type, mutant, or expanded gene peptides may also be used as disease diagnostics and prognostics. Such diagnostic methods, may be used to detect abnormalities in the level of gene protein expression, or abnormalities in the structure and/or tissue, cellular, or subcellular location of fingerprint gene protein. Structural differences may include, for example, differences in the size, electronegativity, or antigenicity of the mutant fingerprint gene protein relative to the normal fingerprint gene protein.

Protein from the tissue or cell type to be analyzed may easily be detected or isolated using techniques which are well known to those of skill in the art, including but not limited to western blot analysis. For a detailed explanation of methods for carrying out western blot analysis, see Sambrook, et al. (1989) supra, at Chapter 18. The protein detection and isolation methods employed herein may also be such as those described in Harlow and Lane, for example, (Antibodies: A Laboratory Manual, Cold Spring Harbor Laboratory Press, Cold Spring Harbor, N.Y. (1988)).

Preferred diagnostic methods for the detection of wild type, mutant, or expanded gene peptide molecules may involve, for example, immunoassays wherein fingerprint gene peptides are detected by their interaction with an anti-fingerprint gene-specific peptide antibody.

For example, antibodies, or fragments of antibodies useful in the present disclosure may be used to quantitatively or qualitatively detect the presence of wild type, mutant, or expanded gene peptides. This can be accomplished, for example, by immunofluorescence techniques employing a fluorescently labeled antibody (see below) coupled with light microscopic, flow cytometric, or fluorimetric detection. Such techniques are especially preferred if the fingerprint gene peptides are expressed on the cell surface.

The antibodies (or fragments thereof) useful in the present disclosure may, additionally, be employed histologically, as in immunofluorescence or immunoelectron microscopy, for in situ detection of fingerprint gene peptides. In situ detection may be accomplished by removing a histological specimen from a patient, and applying thereto a labeled antibody of the present disclosure. The antibody (or fragment) is preferably applied by overlaying the labeled antibody (or fragment) onto a biological sample. Through the use of such a procedure, it is possible to determine not only the presence of the fingerprint gene peptides, but also their distribution in the examined tissue. Using the present disclosure, those of ordinary skill will readily perceive that any of a wide variety of histological methods (such as staining procedures) can be modified in order to achieve such in situ detection.

Immunoassays for wild type, mutant, or expanded fingerprint gene peptides typically comprise incubating a biological sample, such as a biological fluid, a tissue extract, freshly harvested cells, or cells which have been incubated in tissue culture, in the presence of a detectably labeled antibody capable of identifying fingerprint gene peptides, and detecting the bound antibody by any of a number of techniques well known in the art.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled gene-specific antibody. The solid phase support may then be washed with the buffer a second time to remove unbound antibody. The amount of bound label on solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present disclosure. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot of anti-wild type, -mutant, or -expanded fingerprint gene peptide antibody may be determined according to well known methods. Those skilled in the art will be able to determine operative and optimal assay conditions for each determination by employing routine experimentation.

One of the ways in which the gene peptide-specific antibody can be detectably labeled is by linking the same to an enzyme and using it in an enzyme immunoassay (EIA) (Voller, Ric Clin Lab, 8:289-98 (1978) [“The Enzyme Linked Immunosorbent Assay (ELISA)”, Diagnostic Horizons 2:1-7, 1978, Microbiological Associates Quarterly Publication, Walkersville, Md.]; Voller, et al., J. Clin. Pathol., 31:507-20 (1978); Butler, Meth. Enzymol., 73:482-523 (1981); Maggio (ed.), Enzyme Immunoassay, CRC Press, Boca Raton, Fla. (1980); Ishikawa, et al., (eds.) Enzyme Immunoassay Igaku-Shoin, Tokyo (1981)). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect fingerprint gene wild type, mutant, or expanded peptides through the use of a radioimmunoassay (RIA) (see, e.g., Weintraub, B., Principles of Radioimmunoassays, Seventh Training Course on Radioligand Assay Techniques, The Endocrine Society, March, 1986). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediamine-tetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present disclosure. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Throughout this application, various publications, patents, and published patent applications are referred to by an identifying citation. The disclosures of these publications, patents and published patent specifications referenced in this application are hereby incorporated by reference into the present disclosure to more fully describe the state of the art to which this disclosure pertains.

The following examples are intended only to illustrate the present disclosure and should in no way be construed as limiting the subject disclosure.

### EXAMPLES

### Example 1

### Knockout of Target T243 and Analysis of Homozygous Knockout Mutant Mice

In one embodiment, the targeting construct was introduced into ES cells derived from the 129/OlaHsd mouse substrain to generate chimeric mice. The F1 mice were generated by breeding with C57BL/6 females, and the resultant FINO heterozygotes were backcrossed to C57BL/6 mice to generate F1N1 heterozygotes. The F2N1 homozygous mutant mice were produced by intercrossing F1N1 heterozygous males and females.

Genomic DNA from the recombinant ES line was assayed for homologous recombination using polymerase chain reactions (PCRs). Both 5′ PCR reconfirmation and 3′ PCR reconfirmation was performed. The method employed a gene-specific (GS) primer, which was outside of and adjacent to the targeting vector arm, paired in succession with one of three primers in the insertion fragment. The “DNA sample control” employed a primer pair intended to amplify a fragment from a non-targeted genomic locus. The “positive control” employed the GS primer paired with a primer at the other end of the arm. Amplified DNA fragments were visualized by ethidium bromide staining following agarose gel electrophoresis and matched the expected product sizes, in base pairs (bp).

In addition, genomic DNA isolated from both the parent ES line and the recombinant ES line was digested with restriction enzymes (determined to cut outside of the construct arms). The DNA was analyzed by Southern hybridization, and probed with a radiolabeled DNA fragment that hybridized outside of and adjacent to the construct arm. The parent ES line (negative control) showed bands representing the endogenous (wild-type) allele. In contrast, the recombinant ES line showed an additional band representing the targeted allele from the expected homologous recombination event.

The initial germ line F1 (129×C57BL/6) mice were genotyped by either PCR or Southern blot analysis. For both PCR and Southern analysis, oligonucleotides or probes were selected outside the targeting vector to avoid detecting vector alone and to confirm the homologous recombination event. F2 generation mice [F1(129×C57BL/6)×F1 (129×C57BL/6)] were subsequently genotyped by PCR analysis. Gene expression analysis was performed using the knocked-in reporter gene and RT-PCR.

### Example 2

### Transgenic Mice Overexpressing T243

Production of Transgenic Mice by Pronuclear Injection

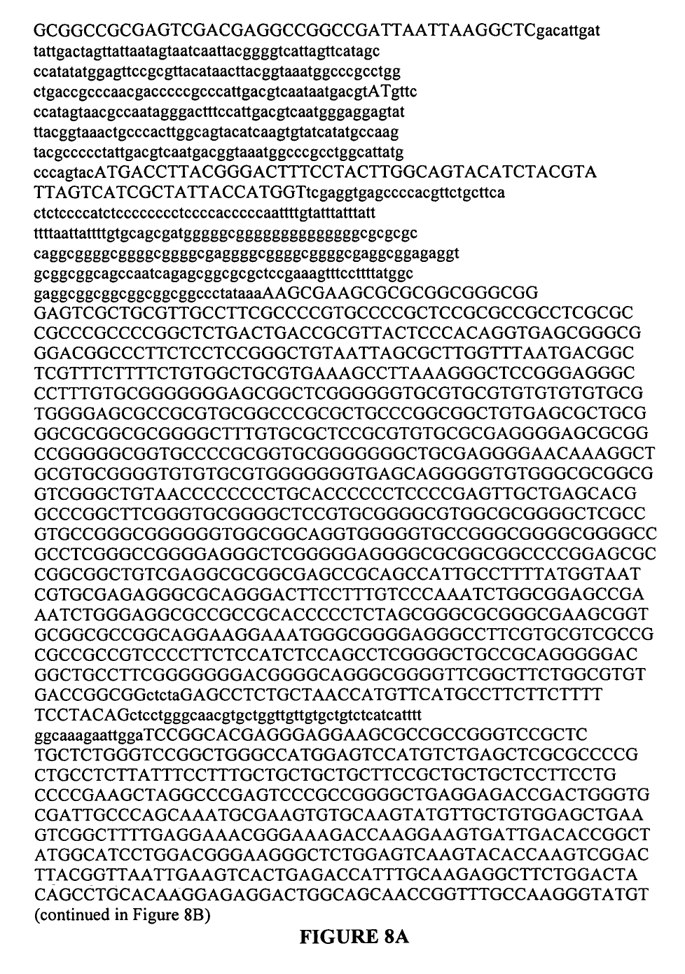

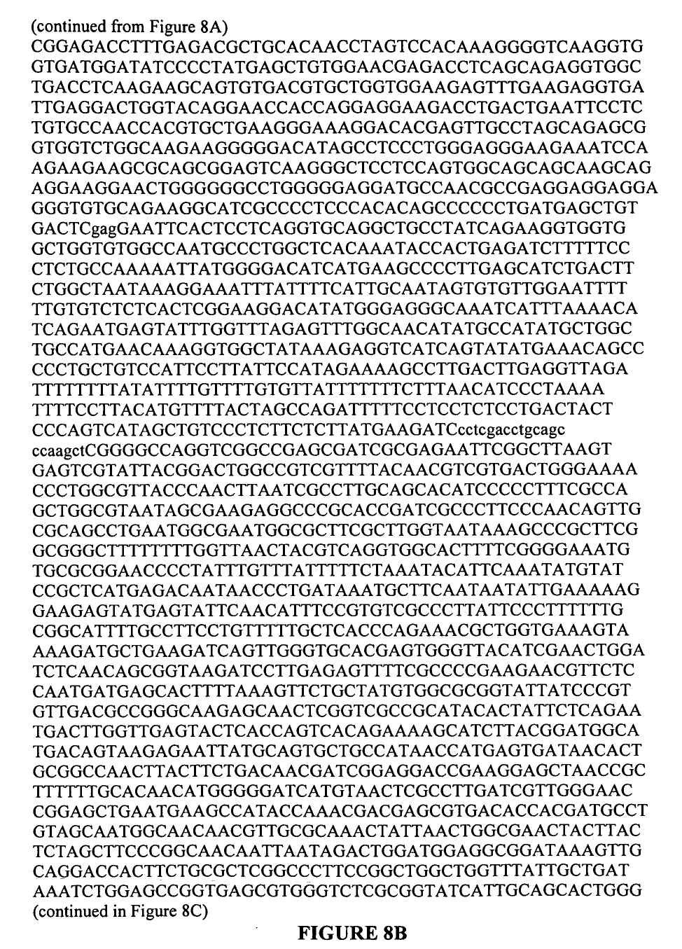

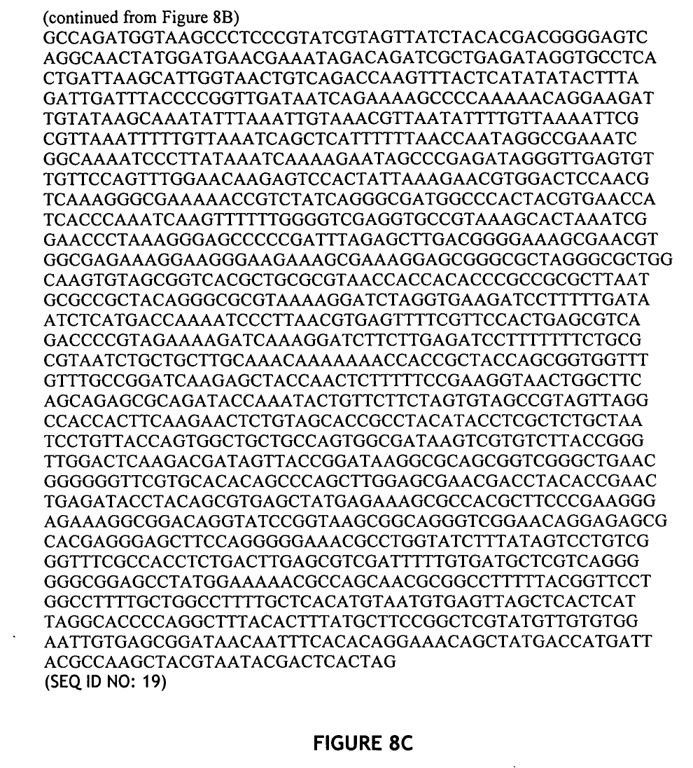

To investigate the role of T243, two lines of transgenic mice were generated by pronuclear injection. Specifically, transgenic mice comprising a chicken beta actin promoter to drive high level expression of the mouse T243 cDNA were created. The cDNA was a full length T243 cDNA that did not have any additional fusion tags. More particularly, a T243-specific targeting construct based on SEQ ID NO: 19 (see FIGS. 8A-C) was created.

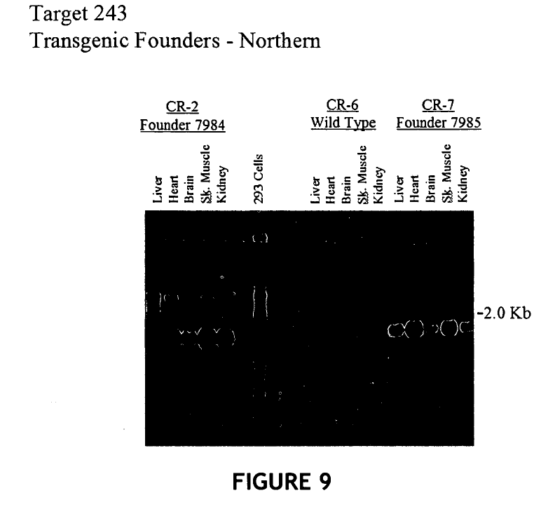

The targeting vector containing the chicken beta actin promoter driving the T243 cDNA was digested and gel-purified to remove the plasmid vector backbone sequences. The targeting construct was microinjected into the male pronucleus of a fertilized zygote. Embryos were transferred into host recipients for gestation. After weaning, tail biopsies were screened for the presence of the transgene. Founders, containing the transgene, were bred to C57BL/6 mice ensure maintenance of the line through the germline. Two lines containing the transgene, from Founder 7984 (CR-2) and Founder 7985 (CR-7) were expanded by breeding for analysis. Thus two high expressing lines were generated as shown in Northern blot analysis in FIG. 9.

### Example 3

### Expression Analysis

RT-PCR Expression. Total RNA was isolated from the organs or tissues from adult C57BL/6 wild-type mice. RNA was DNaseI treated, and reverse transcribed using random primers. The resulting cDNA was checked for the absence of genomic contamination using primers specific to non-transcribed genomic mouse DNA. cDNAs were balanced for concentration using HPRT primers.

RNA transcripts were detectable in all tissues analyzed as shown in Table 1.

TABLE 1
RT-PCR gel
Test Date
Jul. 23, 2001 14:16

| | | | |
|---|---|---|---|
| Gene | 243 | skin | weak |
| ES Cell Line | 242 | gallbladder | weak |
| whole brain | weak | urinary bladder | weak |
| cortex | weak | pituitary gland | weak |
| subcortical region | weak | adrenal gland | weak |
| cerebellum | weak | salivary gland | medium |
| brainstem | weak | skeletal muscle | weak |
| olfactory bulb | weak | tongue | weak |
| spinal cord | weak | stomach | medium |
| eyes | weak | small intestine | weak |
| harderian gland | medium | large intestine | weak |
| heart | medium | cecum | weak |
| lung | medium | testis | medium |
| liver | medium | epididymis | weak |
| pancreas | strong | seminal vesicle | weak |
| kidney | medium | coagulating gland | medium |
| spleen | medium | prostate | weak |
| thymus | weak | ovary | medium |
| lymph nodes | weak | uterus | weak |
| bone marrow | weak | white fat | weak |

T243 is widely expressed in multiple tissues. The highest level of expression, deduced by rtPCR analysis, is in pancreas.

### Example 4

### Physical Examination

A complete physical examination was performed on each mouse. Mice were first observed in their home cages for a number of general characteristics including activity level, behavior toward siblings, posture, grooming, breathing pattern and sounds, and movement. General body condition and size were noted as well identifying characteristics including coat color, belly color, and eye color. Following a visual inspection of the mouse in the cage, the mouse was handled for a detailed, stepwise examination. The head was examined first, including eyes, ears, and nose, noting any discharge, malformations, or other abnormalities. Lymph nodes and glands of the head and neck were palpated. Skin, hair coat, axial and appendicular skeleton, and abdomen were also examined. The limbs and torso were examined visually and palpated for masses, malformations or other abnormalities. The anogenital region was examined for discharges, staining of hair, or other changes. If the mouse defecates during the examination, the feces were assessed for color and consistency. Abnormal behavior, movement, or physical changes may indicate abnormalities in general health, growth, metabolism, motor reflexes, sensory systems, or development of the central nervous system. Mouse body weights and body lengths were measured at various days of age. Mouse metrics data is shown in Table 2.

When compared to wild-type control mice (+/+) and heterozygous mice (−/+), homozygous mice (−/−) exhibited significantly decreased body weight, body length, and body weight to body length ratios.

TABLE 2
Mouse Metrics, F2N0 Mice

| | | Age at Test | | body weight | body length | body weight/ |
|---|---|---|---|---|---|---|
| Genotype | Gender | days | n | (g) | (cm) | body length |
| −/− | Female | 5 +/− 2 | 8 | 1.68 +/− 0.4 | 3.22 +/− 0.38 | 0.52 +/− 0.08 |
| −/+ | Female | 5 +/− 2 | 35 | 3.59 +/− 1.50 | 4.12 +/− 0.56 | 0.84 +/− 0.25 |
| +/+ | Female | 6 +/− 2 | 42 | 3.57 +/− 1.55 | 4.13 +/− 0.61 | 0.83 +/− 0.26 |
| −/− | Female | 13 +/− 2 | 7 | 3.11 +/− 0.73 | 4.22 +/− 0.56 | 0.73 +/− 0.08 |
| −/+ | Female | 13 +/− 2 | 37 | 8.65 +/− 2.02 | 5.86 +/− 0.55 | 1.46 +/− 0.25 |
| +/+ | Female | 13 +/− 2 | 45 | 8.03 +/− 1.82 | 5.77 +/− 0.54 | 1.38 +/− 0.22 |
| −/− | Female | 19 +/− 2 | 9 | 3.23 +/− 0.46 | 4.83 +/− 0.43 | 0.67 +/− 0.06 |
| −/+ | Female | 20 +/− 2 | 58 | 10.21 +/− 2.25 | 6.58 +/− 0.48 | 1.54 +/− 0.26 |
| +/+ | Female | 20 +/− 2 | 52 | 10.13 +/− 1.59 | 6.55 +/− 0.32 | 1.54 +/− 0.20 |
| −/+ | Female | 28 +/− 4 | 33 | 13.61 +/− 3.41 | 7.30 +/− 0.78 | 1.84 +/− 0.32 |
| +/+ | Female | 26 +/− 2 | 17 | 13.20 +/− 1.53 | 7.08 +/− 0.29 | 1.86 +/− 0.19 |
| −/+ | Female | 73 +/− 3 | 5 | 21.16 +/− 3.88 | 9.20 +/− 0.48 | 2.29 +/− 0.34 |
| +/+ | Female | 70 +/− 4 | 6 | 23.40 +/− 2.42 | 9.38 +/− 0.21 | 2.50 +/− 0.21 |
| −/+ | Male | 6 +/− 2 | 65 | 3.65 +/− 1.51 | 4.14 +/− 0.59 | 0.85 +/− 0.25 |
| +/+ | Male | 6 +/− 2 | 10 | 3.96 +/− 1.32 | 4.35 +/− 0.51 | 0.89 +/− 0.21 |
| −/− | Male | 14 +/− 2 | 14 | 4.73 +/− 0.31 | 4.91 +/− 0.29 | 0.96 +/− 0.03 |
| −/+ | Male | 13 +/− 2 | 59 | 7.69 +/− 1.88 | 5.65 +/− 0.55 | 1.34 +/− 0.24 |
| +/+ | Male | 14 +/− 2 | 36 | 7.84 +/− 2.08 | 5.69 +/− 0.53 | 1.36 +/− 0.25 |
| −/− | Male | 20 +/− 2 | 19 | 4.30 +/− 0.59 | 5.39 +/− 0.20 | 0.80 +/− 0.11 |
| −/+ | Male | 20 +/− 2 | 83 | 9.48 +/− 2.4S | 6.37 +/− 0.59 | 1.46 +/− 0.29 |
| +/+ | Male | 20 +/− 2 | 50 | 9.48 +/− 2.63 | 6.37 +/− 0.55 | 1.47 +/− 0.30 |

In cage observation, homozygous mice were initially hyperactive as compared to normal littermates and had very dry skin. By about 15-17 days, homozygous knockout mice began to appear increasingly unstable and lethargic; by about 19-21 days, homozygotes showed signs of shivering and impending death. Homozygous knockout mice which were not found dead, were sacrificed at approximately 23-25 days for further analysis. Homozygous pups were approximately the same size or slightly smaller than wild type or heterozygous littermates at birth. With age, however, both weight gain and lengthwise growth were markedly decreased in homozygous knockout pups. By 15-17 days, homozygotes began to lose weight, such weight loss continuing until death at approximately 3 weeks.

### Example 5

### Necropsy

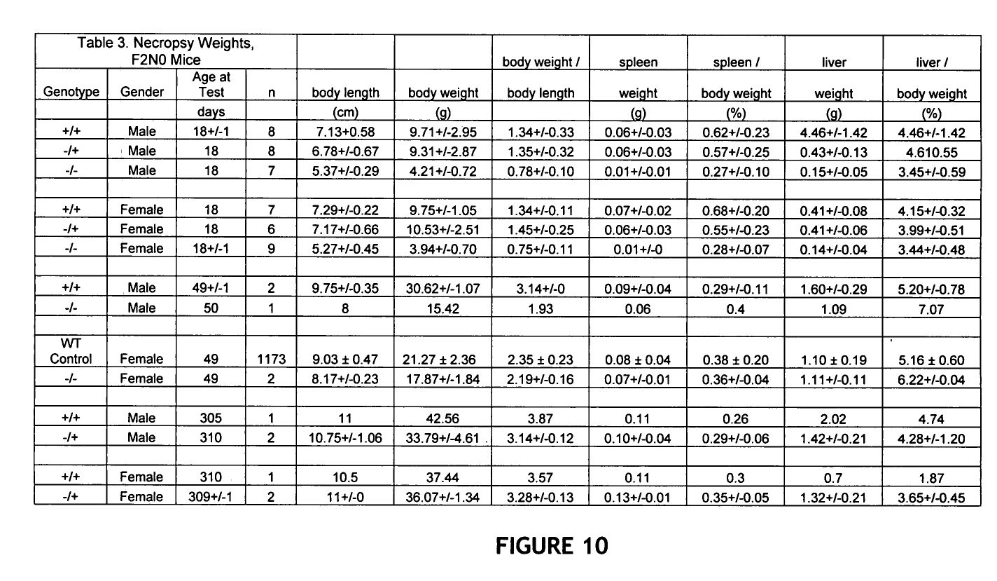

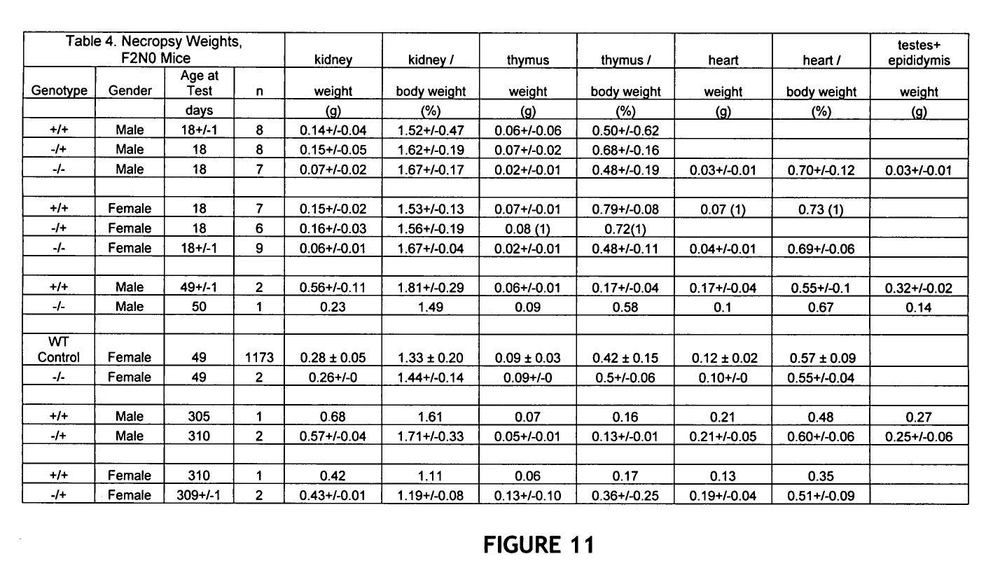

Necropsy was performed on mice following deep general anesthesia, cardiac puncture for terminal blood collection, and euthanasia. Body lengths and body weights were recorded for each mouse. The necropsy included detailed examination of the whole mouse, the skinned carcass, skeleton, and all major organ systems. Lesions in organs and tissues were noted during the examination. Designated organs, from which extraneous fat and connective tissue have been removed, were weighed on a balance, and the weights were recorded. Weights were obtained for the following organs: heart, liver, spleen, thymus, kidneys, and testes/epididymides. Certain necropsy weight results are shown in FIGS. 10 and 11 (Tables 3 and 4). When compared to wild-type control mice (+/+) and heterozygous mice (−/+), homozygous mice exhibited decreased body length, decreased body weight, decreased body weight to body length ratio, decreased spleen weight, decreased spleen weight to body weight ratio, decreased liver weight, decreased kidney weight, and decreased thymus weight. Necropsy was performed on 6 homozygous mutants (4 female, 2 male) and 3 controls (2 female, 1 male). Significant differences attributable to the T243 mutation were observed in bone and kidney tissues.

Mutant mice had abnormal cartilage and a generalized reduction of bone formation. Specifically, shortening of both the axial and appendicular skeleton was observed. Proximal and distal bones of the limbs were proportionally shortened and joint cartilage lacked alcian blue staining. The distal femur had a thin growth plate and thin to absent epiphyseal cartilage. A single mutant mouse had a microfracture extending diagonally from the cortex through the metaphysis into the physis (suggestive of growth plate fragility). Within the physes of all mutant mice, chondrocyte columns in the proliferating and hypertrophic zones were short. Cartilaginous spicules within the metaphysis were short and widely spaced. Occasional spicules were haphazardly oriented. Osteoblasts were abundant and frequently piled up along cartilaginous spicules. Epiphyseal cartilage was thin and often replaced by fibrous connective tissue. The epiphyseal surface showed decreased staining with alcian blue. Cartilage at the epiphyseal/physeal junction was slightly flared with an irregular, prominent edge that overhung the physis.

Mutant sternebrae were found to be irregular. Growth plates were either lacking or discontinuous. Large, irregular islands of cartilage extended into the shaft of the sternebra and occasionally had secondary ossification centers. Edges of the cartilage were flared.

Based on alcian blue stains, vertebral bodies were variably ossified. Some were small and predominantly cartilaginous with irregular and thin growth plates showing tapered lateral processes.

All of the mutant mice had dysplastic changes in both kidneys that were most prominent in the corticomedullary junction and to a lesser extent in the cortex. The kidneys were small and lacked normal architecture. The cortex was thin and some glomeruli were subcapsular. Subcapsular glomeruli were small with shrunken, hypercellular glomerular tufts indicating immaturity. The corticomedullary area lacked radiating arcuate vessels and distinct tubule formation. Tubular epithelial cells within the corticomedullary junction were haphazardly arranged into sheets, piles, and clusters. Some tubular epithelial cells were small and darkly basophilic, thus appearing to be regenerative.

### Example 6

### Hematological Analysis

Blood samples were collected via a terminal cardiac puncture in a syringe. About one hundred microliters of each whole blood sample were transferred into tubes pre-filled with EDTA. Approximately 25 microliters of the blood was placed onto a glass slide to prepare a peripheral blood smear. The blood smears were later stained with Wright's Stain that differentially stained white blood cell nuclei, granules and cytoplasm, and allowed the identification of different cell types. The slides were analyzed microscopically by counting and noting each cell type in a total of 100 white blood cells. The percentage of each of the cell types counted was then calculated. Red blood cell morphology was also evaluated.

Microscopic examinations of blood smears were performed to provide accurate differential blood leukocyte counts. The leukocyte differential counts were provided as the percentage composition of each cell type in the blood.

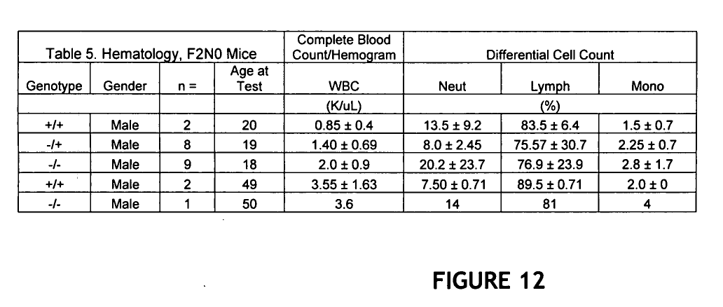

Interesting hematology data are shown in FIG. 12 (Table 5). When compared to wild-type control mice, certain homozygous mice exhibited increased white blood cells (WBC), increased neutrophils, and increased monocytes.

White blood cells (WBC) represents the sum total of the counts of granulocytes, lymphocytes and monocytes per unit volume of whole blood.

Neutrophils, also called granulocytes or segmented neutrophils, are the main defense against infection and antigens. High levels may indicate an active immune system, low levels may indicate a depressed immune system or low production by bone marrow.

Monocytes are useful in fighting infection and are the bodies second line of defense against infection. Monocytes are the largest cells in the blood. Monocytes may be elevated in the case of tissue breakdown, chronic infection, carcinoma, monocytic leukemia, or lymphomas.

### Example 7

### Serum Chemistry

Blood samples were collected via a terminal cardiac puncture in a syringe. One hundred microliters of each whole blood sample was transferred into a tube pre-filled with EDTA. The remainder of the blood sample was converted to serum by centrifugation in a serum tube with a gel separator. Each serum sample was then analyzed as described below. Non-terminal blood samples for aged mice are collected via retro-orbital venous puncture in capillary tubes. This procedure yields approximately 200 uL of whole blood that is either transferred into a serum tube with a gel separator for serum chemistry analysis (see below), or into a tube pre-filled with EDTA for hematology analysis.

The serum was analyzed for the following parameters: alanine aminotransferase, albumin, alkaline phosphatase, aspartate transferase, bicarbonate, total bilirubin, blood urea nitrogen, calcium, chloride, cholesterol, creatine kinase, creatinine, globulin, glucose, high density lipoproteins (HDL), lactate dehydrogenase, low density lipoproteins (LDL), osmolality, phosphorus, potassium, total protein, sodium, and triglycerides.

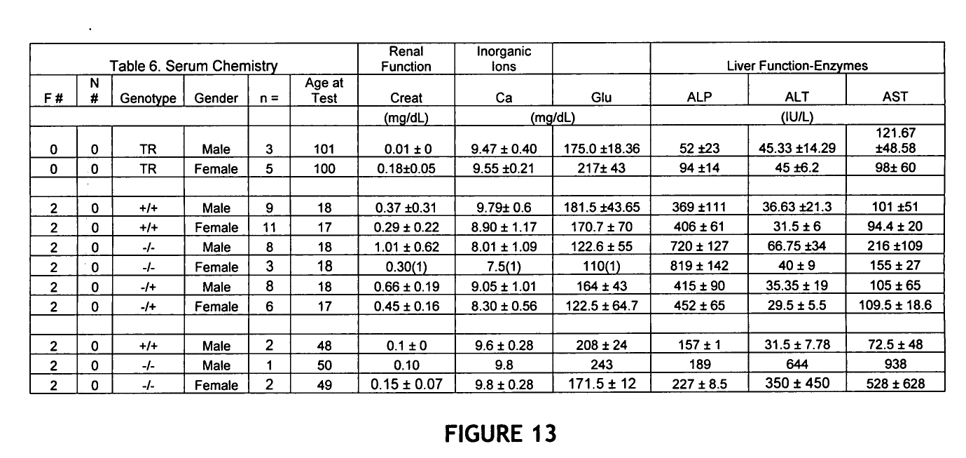

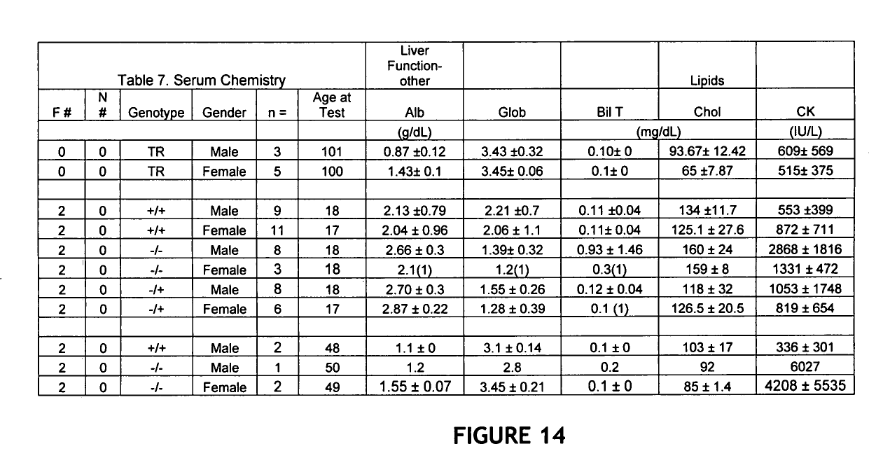

Results for homozygous and heterozygous mice were compared to wild-type control mice with same ES parent, gender, F, N, and age. For all data collected, two-tailed pair-wise statistical significance was established using a Student t-test. Statistical significance was defined as P≦0.05. Data were considered statistically significant if 1-p vs. wild-type control value was ≧0.95. Statistically significant serum chemistry phenotypes are displayed in bold in FIGS. 13 and 14 (Tables 6 and 7); average values, plus or minus the standard deviation, are shown for F2N0 homozygous (−/−), heterozygous (−/+), wild type control mice (+/+) and transgenic mice (TR).

When compared to wild-type control mice, certain homozygous mice exhibited increased creatinine, decreased calcium (Ca), decreased glucose, increased alkaline phosphatase (ALP), increased alanine aminotransferase (ALT), increased aspartate aminotransferase (AST), increased albumin, decreased globulin, increased total bilirubin (Bil T), increased cholesterol, and increased creatine kinase (CK).

Calcium (Ca) is the most abundant mineral in the body. Calcium is involved in bone metabolism, protein absorption, fat transfer muscular contraction, transmission of nerve impulses, blood clotting and cardiac function. Serum calcium is sensitive to other elements such as magnesium, iron, phophorus, as well as hormonal activity, vitamin D levels, and alkalinity and acidity. Hypercalcemia is seen in malignant neoplasms, primary and tertiary hyperparathyroidism, sarcoidosis, vitamin D intoxication, milk-alkali syndrome, Paget's disease of bone, thyrotoxicosis, acromegaly, and diuretic phase of tubular necrosis. Hypocalcemia must be interpreted in relation to serum albumin concentration. True decrease in calcium occurs in hypoparathyroidism, vitamin D deficiency, chronic renal failure, magnesium deficiency, and acute pancreatitis.

Serum glucose results from the digestion of carbohydrates and the conversion of glycogen by the liver. Glucose is the primary energy source for most cells. It is regulated by insulin, glucagon, thyroid hormone, liver enzymes and adrenal hormones. Increased fasting serum glucose may be indicative of diabetes mellitis.

Alkaline phosphatase (ALP) is produced by the cells of bone, liver, kidney, intestine and placenta. ALP is sometimes used as a tumor marker and is elevated in bone injury, pregnancy or skeletal growth.

Alanine aminotransferase (ALT) is a liver enzyme which also occurs in the kidneys, heart, and skeletal muscles. ALT is one of two main liver function blood serum tests. ALT is a marker of acute liver damage and is slightly to moderately elevated in any condition that produces acute liver cell injury, e.g. active cirrhosis and hepatitis.

Aspartate aminotransferase (AST) is one of two main liver function blood serum tests. AST levels fluctuate with the extent of cellular necrosis (cell death). Increased AST levels may be seen in any condition involving necrosis of hepatocytes, myocardial cells, or skeletal muscle cells. AST level may be used to help detect a recent myocardial infarction and in differential diagnosis of acute hepatic disease.

Cholesterol is a structural component of cell membrane and plasma lipoproteins and is essential in the synthesis of steroid hormones, glucocorticoids, and bile acids. Low levels of cholesterol are seen in immune compromised patients, poor dietary habits, malabsorption, and liver or kidney disease.

Creatine kinase (CK) is an enzyme found in muscle, brain, and other tissues that catalyzes the transfer of a phosphate group from adenosine triphosphate to creatine to form phosphocreatine. Increased CK may be used to help diagnose myocardial infarction and muscle damage in progressive muscular dystrophy and sickle cell anemia.

Globulin is important in immune responses. Elevated levels may be seen in chronic infection, liver disease, rheumatoid arthritis, myelomas, and lupus. Low levels are seen in immune compromised patients, poor dietary habits, malabsorption and liver or kidney disease.

Serum albumin is a major serum protein. It is synthesized in the liver from amino acids in the diet. Albumin functions to help maintain osmotic pressure, nutrient transport, and waste removal. High levels may be seen rarely in liver disease, shock, dehydration, or multiple myeloma. Low levels may be seen associated with poor diet, diarrhea, fever, infection, liver disease, inadequate iron, burns, edema, or hypocalcemia.

Creatinine is a waste product of muscle metabolism. Low creatinine levels may be seen in cases of kidney damage, protein starvation, liver disease and pregnancy. Creatinine increase is seen in renal functional impairment, kidney disease, and muscle degeneration.

Serum total bilirubin is increased in hepatocellular damage from various causes, biliary tract obstruction, hemolysis, neonatal jaundice, fructose intolerance, Crigler-Najjar syndrome, Gilbert's disease, and Dubin-Johnson syndrome.

### Example 8

### Densitometric Analysis

Mice were euthanized and analyzed using a PIXImus™ densitometer. An x-ray source exposed the mice to a beam of both high and low energy x-rays. The ratio of attenuation of the high and low energies allowed the separation of bone from soft tissue, and, from within the tissue samples, lean and fat. Densitometric data including Bone Mineral Density (BMD presented as g/cm²), Bone Mineral Content (BMC in g), bone and tissue area, total tissue mass, and fat as a percent of body soft tissue (presented as fat %) were obtained and recorded.

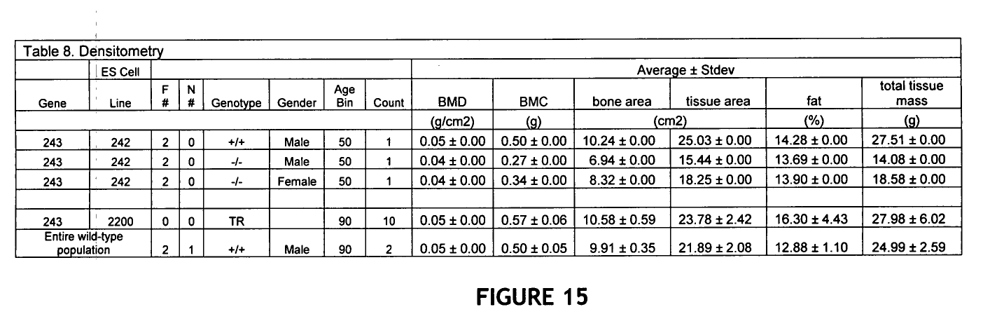

Data for densitometry of homozygous (−/−), heterozygous (−/+), wild-type control mice (+/+) and transgenic mice overexpressing T243 (TR) are shown in FIG. 15 (Table 8).

Homozygous mice exhibited decreased bone mineral density, decreased bone mineral content, decreased fat tissue mass, and decreased total tissue mass, when compared to wild-type control mice.

Transgenic mice (TR), overexpressing T243, exhibited increased bone mineral content (BMC), and increased bone area when compared to wild-type control mice (+/+) as shown in FIG. 15 (Table 8).

Generally, mice with decreased expression of T243 exhibited decreased bone-density, while mice with increased expression exhibited increased bone density.

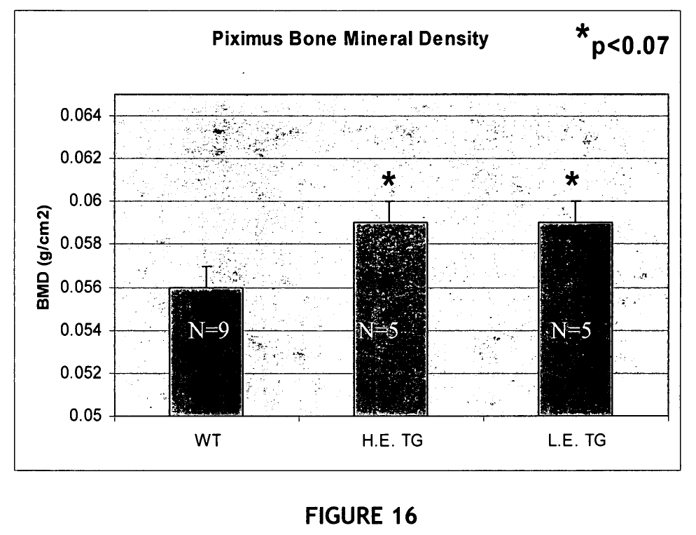

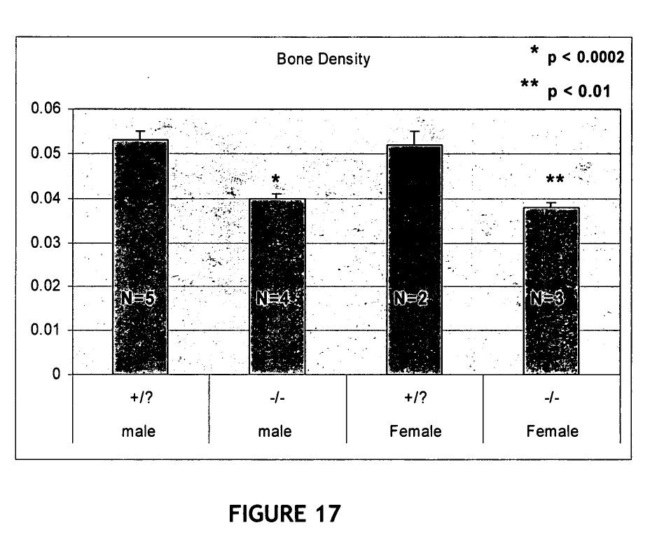

Ovariectomy to deplete female mice of estrogen was performed on high expressing transgenic mice (H.E. TG), low expressing TG (L.E. TG) and wild-type control mice as shown in FIG. 16. In the ovariectomy challenge, transgenic mice over expressing T243 exhibited about 7% greater bone mineral density than wild-type control mice after 6 weeks of estrogen depletion. In another experiment, homozygous mice backcrossed to CDI (+/?) survived to adulthood and exhibited about 20% increased bone mineral density when compared to homozygous mice (−/−) as shown in FIG. 17.

### Example 9

### Behavioral Analysis—Rotarod Test

The Accelerating Rotarod was used to screen for motor coordination, balance and ataxia phenotypes. Mice were allowed to move about on their wire-cage top for 30 seconds prior to testing to ensure awareness. Mice were placed on the stationary rod, facing away from the experimenter. The “speed profile” programs the rotarod to reach 60 rpm after six minutes. A photobeam was broken when the animal fell, which stopped the test clock for that chamber. The animals were tested over three trials with a 20-minute rest period between trials, after which the mice were returned to fresh cages. The data was analyzed to determine the average speed of the rotating rod at the fall time over the three trials. A decrease in the speed of the rotating rod at the time of fall compared to wild-types indicated decreased motor coordination possibly due to a motor neuron or inner ear disorder.

### Example 10

### Behavioral Analysis—Startle Test

The startle test screens for changes in the basic fundamental nervous system or muscle-related functions. The startle reflex is a short-latency response of the skeletal musculature elicited by a sudden auditory stimulus. This includes changes in 1) hearing—auditory processing; 2) sensory and motor processing—related to the auditory circuit and culminating in a motor related output; 3) global sensory changes; and motor abnormalities, including skeletal muscle or motor neuron related changes.

The startle test also screens for higher level cognitive functions. The startle reflex can be modulated by negative affective states like fear or stress. The cognitive changes include: 1) sensorimotor processing such as sensorimotor gating changes related to schizophrenia; 2) attention disorders; 3) anxiety disorders; and 4) thought disturbance disorders.

The mice were tested in a San Diego Instruments SR-LAB sound response chamber. Each mouse was exposed to 9 stimulus types that were repeated in pseudo-random order ten times during the course of the entire 25-minute test. The stimulus types in decibels were: p85, p90, p100, p110, p120, pp85 p120, pp90p110, pp90p120; where p=40 msec pulse, pp=20 msec prepulse. The length of time between a prepulse and a pulse was 100 msec (onset to onset). The mean Vmax of the ten repetitions for each trial type was computed for each mouse.

The % prepulse inhibition (PPI) compared to p120 or p110 alone is computed for each mouse at three prepulse levels from the mean Vmax values and this is presented in a chart. This is computed by determining the mean “p120”, “pp85p120”, “pp90p110”, and “pp90p120” value for each mouse and then producing the ratios of % inhibition.
PPI85=((p120-pp85p120)/p120)×100). Example

### Example 11

### Behavioral Analysis—Hot Plate Test

The hot plate analgesia test was designed to indicate an animal's sensitivity to a painful stimulus. The mice were placed on a hot plate of about 55.5° C., one at a time, and latency of the mice to pick up and lick or fan a hindpaw was recorded. A built-in timer was started as soon as the subjects were placed on the hot plate surface. The timer was stopped the instant the animal lifted its paw from the plate, reacting to the discomfort. Animal reaction time was a measurement of the animal's resistance to pain. The time points to hindpaw licking or fanning, up to a maximum of about 60-seconds, was recorded. Once the behavior was observed, the animal was immediately removed from the hot plate to prevent discomfort or injury.

### Example 12

### Behavioral Analysis—Tail Flick Test

The tail-flick test is a test of acute nociception in which a high-intensity thermal stimulus is directed to the tail of the mouse. The time from onset of stimulation to a rapid flick/withdrawal from the heat source is recorded. This test produces a simple nociceptive reflex response that is an involuntary spinally mediated flexion reflex.

### Example 13

### Behavioral Analysis—Open Field Test

The Open Field Test was used to examine overall locomotion and anxiety levels in mice. Increases or decreases in total distance traveled over the test time are an indication of hyperactivity or hypoactivity, respectively.

The open field provides a novel environment that creates an approach-avoidance conflict situation in which the animal desires to explore, yet instinctively seeks to protect itself. The chamber is lighted in the center and has no places to hide other than the corners. A normal mouse typically spends more time in the corners and around the periphery than it does in the center. Normal mice however, will venture into the central regions as they explore the chamber. Anxious mice spend most of their time in the corners, with almost no exploration of the center, whereas bold mice travel more, and show less preference for the periphery versus the central regions of the chamber.

Each mouse was placed gently in the center of its assigned chamber. Tests were conducted for 10 minutes, with the experimenter out of the animals' sight. Immediately following the test session, the fecal boli were counted for each subject: increased boli are also an indication of anxiety. Activity of individual mice was recorded for the 10-minute test session and monitored by photobeam breaks in the x-, y- and z-axes. Measurements taken included total distance traveled, percent of session time spent in the central region of the test apparatus, and average velocity during the ambulatory episodes. Increases or decreases in total distance traveled over the test time indicate hyperactivity or hypoactivity, respectively. Alterations in the regional distribution of movement indicates anxiety phenotypes, i.e., increased anxiety if there is a decrease in the time spent in the central region.

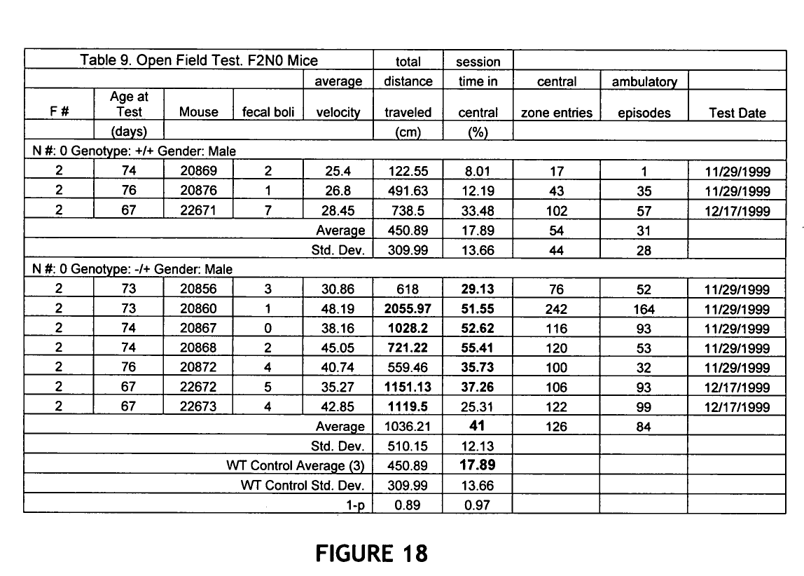

Interesting open field test data for heterozygous mice (−/+) and wild-type control mice at about 72 days of age are shown in FIG. 18 (Table 9). When compared to wild-type control mice, heterozygous mice exhibited significantly greater session time in the central zone as well as a strong trend to increased total distance traveled. Heterozygous mice thus exhibited hyperactivity in the open field test, when compared to wild-type control mice.

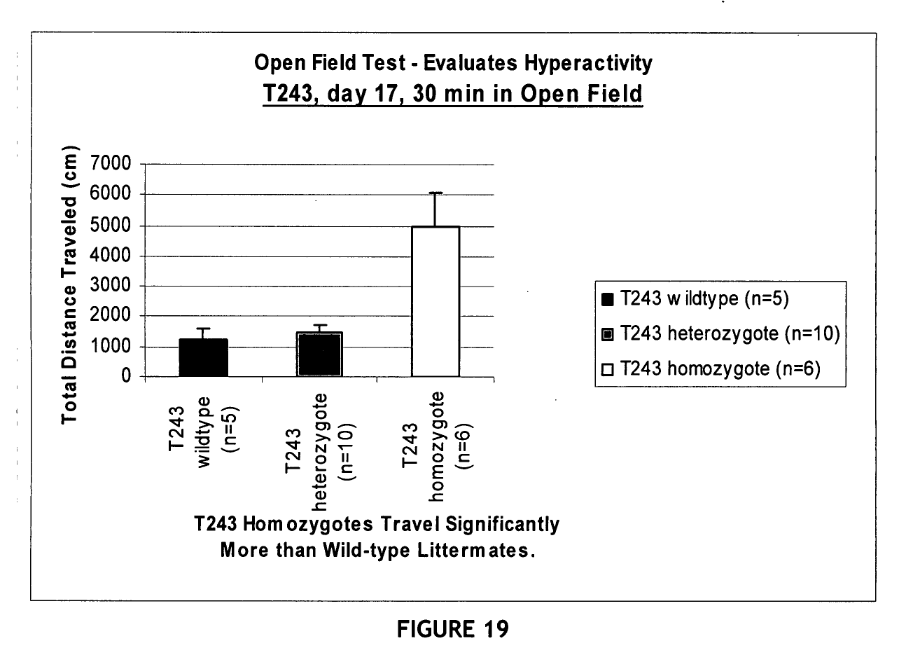

A second open field test was performed to compare homozygous (−/−), heterozygous (−/+), and wild-type control mice (+/+) at about 17 days of age, as shown in FIG. 19. When compared to heterozygous and wild-type control mice, homozygous mice exhibited significantly increased total distance traveled in the open field test. Homozygous mice thus exhibited hyperactivity in the open field test, when compared to wild-type control mice.

### Example 14

### Behavioral Analysis—Metrazol Test

To screen for phenotypes involving changes in seizure susceptibility, the Metrazol Test was be used. About 5 mg/ml of Metrazol was infused through the tail vein of the mouse at a constant rate of about 0.375 ml/min. The infusion caused all mice to experience seizures. Those mice who entered the seizure stage the quickest were thought to be more prone to seizures in general.

The Metrazol test can also be used to screen for phenotypes related to epilepsy. Seven to ten adult wild-type and homozygote males were used. A fresh solution of about 5 mg/ml pentylenetetrazole in approximately 0.9% NaCl was prepared prior to testing. Mice were weighed and loosely held in a restrainer. After exposure to a heat lamp to dilate the tail vein, mice were continuously infused with the pentylenetetrazole solution using a syringe pump set at a constant flow rate. The following stages were recorded: first twitch (sometimes accompanied by a squeak), beginning of the tonic/clonic seizure, tonic extension and survival time. The dose required for each phase was determined and the latency to each phase was determined between genotypes. Alterations in any stage may indicate an overall imbalance in excitatory or inhibitory neurotransmitter levels.

The Metrazol test can also be used to screen for phenotypes related to epilepsy. Seven to ten adult wild-type and homozygote males were used. A fresh solution of about 5 mg/ml pentylenetetrazole in approximately 0.9% NaCl was prepared prior to testing. Mice were weighed and loosely held in a restrainer. After exposure to a heat lamp to dilate the tail vein, mice were continuously infused with the pentylenetetrazole solution using a syringe pump set at a constant flow rate. The following stages were recorded: first twitch (sometimes accompanied by a squeak), beginning of the tonic/clonic seizure, tonic extension and survival time. The dose required for each phase was determined and the latency to each phase was determined between genotypes. Alterations in any stage may indicate an overall imbalance in excitatory or inhibitory neurotransmitter levels.

### Example 15

### Behavioral Analysis—Tail Suspension Test

The tail suspension test is a single-trial test that measures a mouse's propensity towards depression. This method for testing antidepressants in mice was reported by Steru et al., (1985, Psychopharmacology 85(3):367-370) and is widely used as a test for a range of compounds including SSRI's, benzodiazepines, typical and atypical antipsychotics. It is believed that a depressive state can be elicited in laboratory animals by continuously subjecting them to aversive situations over which they have no control. It is reported that a condition of “learned helplessness” is eventually reached.

Mice were suspended on a metal hanger by the tail in an acoustically and visually isolated setting. Total immobility time during the six-minute test period was determined using a computer algorithm based upon measuring the force exerted by the mouse on the metal hanger. An increase in immobility time for mutant mice compared to wild-type mice may indicate increased “depression.” Animals that ceased struggling sooner may be more prone to depression. Studies have shown that the administration of antidepressants prior to testing increases the amount of time that animals struggle.

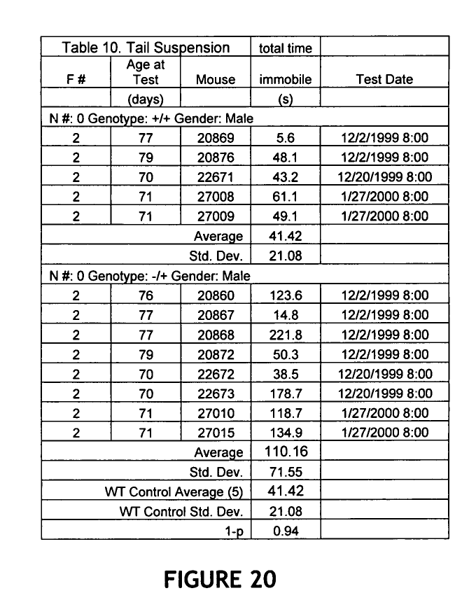

Tail suspension test data are shown in FIG. 20 (Table 10). When compared to wild-type control mice (+/+), heterozygous mice (−/+) exhibited increased total time immobile in the tail suspension test.

### Example 16

### Transgenic Rescue/Overexpression Experiments.

Two lines of transgenic (Tg) mice were generated using a chicken beta actin promoter to drive high level expression of the mouse T243 cDNA as described in Example 2. This was a full length cDNA that did not have any additional fusion tags etc. The two lines of Tg mice were evaluated in several subsequent studies (see below). Characterization of the expression pattern of the transgenic mRNA indicated that both lines generated high level expression in multiple tissues. The expression was estimated to be at least approximately 10-25 fold higher than the endogenous message (endogenous is the faint 2 Kb band in FIG. 9). One transgenic line had higher relative expression levels compared to the other and therefore we designated the lines as H.E. (high expression) and L.E. (low expression). Several advanced studies were performed on the transgenic lines (see below).

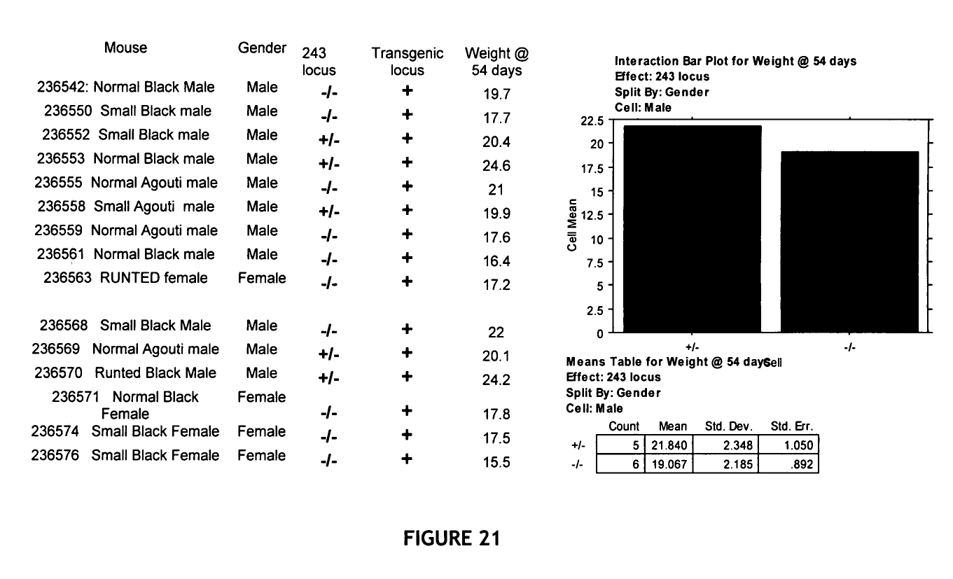

Backcrossing the transgenic lines to the homozygous −/−strain resulted in rescue of the phenotype (FIG. 21; mice at 54 days of age): mice carrying both the Tg allele and the −/−genotype gained weight, and survived to adulthood in a manner that was indistinguishable from +/+littermates. In addition, when analyzed at 25 days of age transgenic mice exhibited no growth, weight, or bone abnormalities and exhibited 100% survival (rescue). The rescued mice were not subjected to any rigorous experimentation beyond this survival analysis.

### Example 17

### Effect on Associated Gene Expression

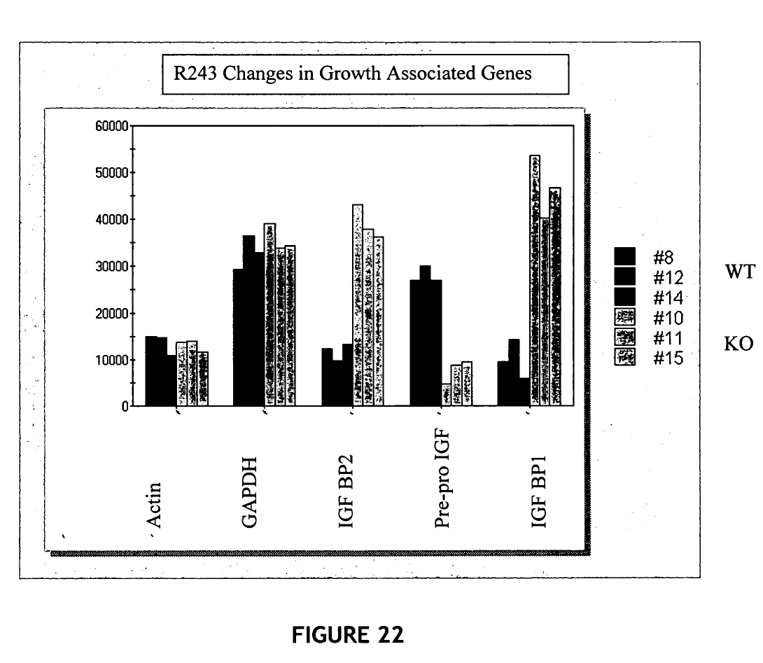

Gene expression profiling was performed using Affymetrix GeneChip® assay with the GeneChip® Murine Genome U74 Set. Homozygous mice (KO, −/−, n=3) were compared to wild-type control mice (WT, +/+, n=3) in terms of expression of growth associated genes by Affymetrix GeneChip analysis, as shown in FIG. 22. Homozygous mice exhibited increased expression of insulin-like growth factor (IGF) BP2, increased IGF BPI, and decreased expression of pre-pro-IGF.

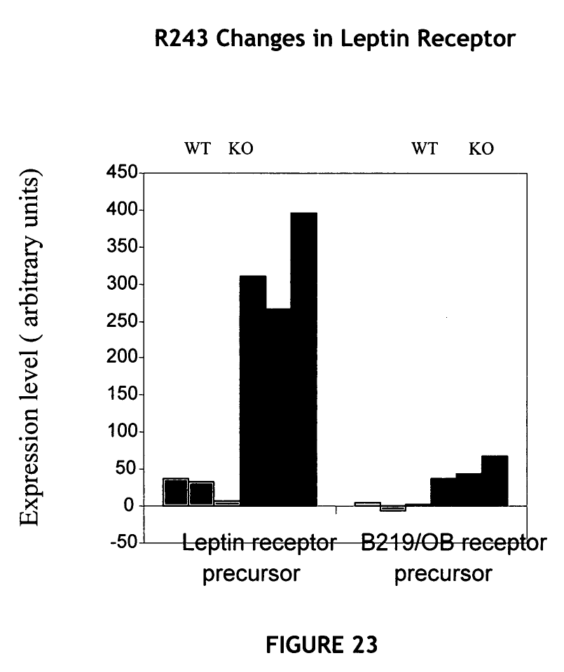

When compared to wild-type control mice, homozygous mice also exhibited increased expression of leptin receptor precursor by Affymetrix gene chip analysis, as shown in FIG. 23. In additional Northern blot analysis, wild-type control mice fasted for 24 to 48 hours exhibited increased expression of leptin receptor isoform A and leptin receptor isoform. The high leptin expression in fasted WT mice was similar to the high leptin expression exhibited by non-fasted T243 homozygous (−/−) mice.

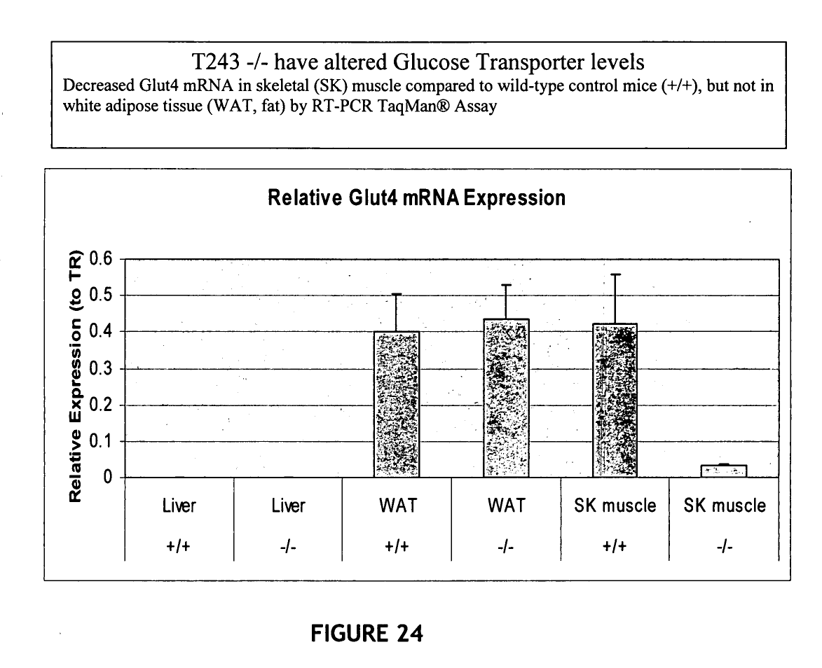

Glucose transporter 4 (Glut4) mRNA expression in skeletal muscle was significantly decreased in homozygous mice (−/−) when compared to wild-type control mice (+/+), by RT-PCR TaqMan® assay, as shown in FIG. 24.

### Example 18

### Liver Glycogen Content

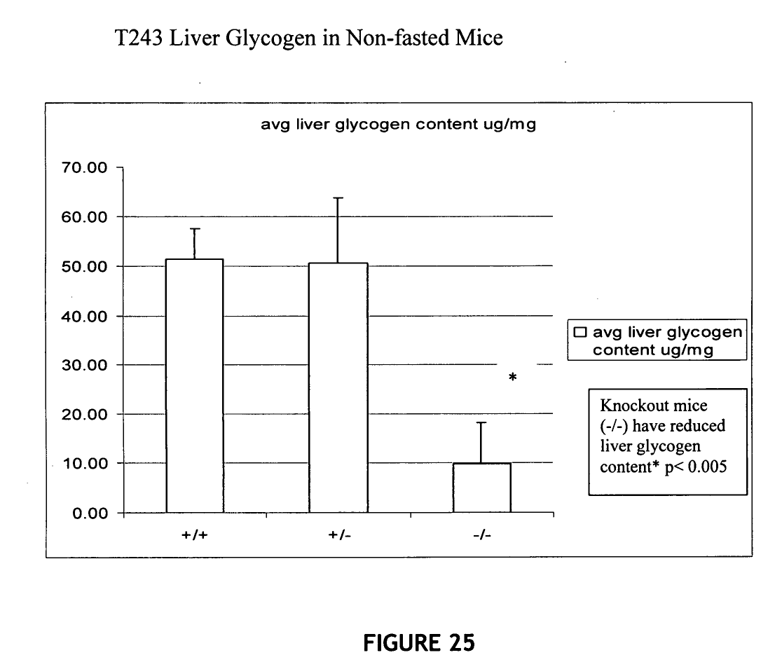

Average liver glycogen content in non-fasted homozygous, heterozygous and wild-type control mice at about 16 days of age was evaluated and data are shown in FIG. 25. When compared to non-fasted heterozygous and wild-type control mice, non-fasted homozygous mice exhibited significantly decreased liver glycogen content.

### Example 19

### Metabolic Screen

Female mice of about 8 weeks old were put on a high fat diet (about 42% calories, Adjusted Calories Diet #88137, Harlan Teklad, Madison, Wis.). Mice were subjected to a Glucose Tolerance Test (GTT), insulin secretion test (IST), and glucose stimulated insulin secretion test (GSIST) about 8 to 10 weeks later and densitometric measurements about 10 weeks later. The body weights and lengths (metrics) were also recorded during the course of high fat diet challenge. For all the data collected, two-tailed unpaired statistical significance was established using a Student t-test. Statistical significance was defined as P<=0.05.

Glucose Tolerance Test (GTT): Mice were fasted for about 3 hours and tail vein blood glucose levels were measured before injection by collecting about 5 to 10 microliters of blood from the tail tip and using glucometers (Glucometer Elite, BayerCorporation, Mishawaka, Ind.). The glucose values were used for time t=0. Mice were weighed at t=0 and glucose was administered orally or by intra-peritoneal injection at a dose of about 2 grams per kilogram of body weight. Plasma glucose concentrations were measured at about 15, 30, 60, 90, and 120 minutes after injection by the same method used to measure basal (t=0) blood glucose.

The glucose levels presented were thought to be representative of the ability of the mouse to secrete insulin in response to elevated glucose levels and the ability of muscle, liver and adipose tissues to uptake glucose.

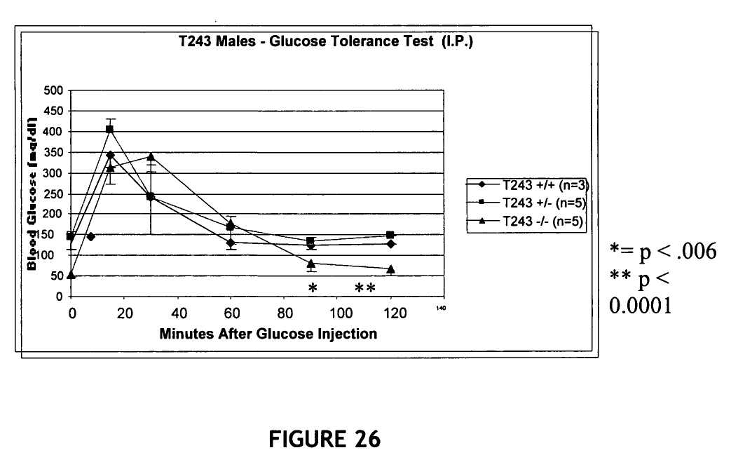

Glucose tolerance test data for male homozygous (−/−), heterozygous (−/+), and wild-type control mice at about 14 days of age are graphed in FIG. 26. When compared to wild-type control mice, homozygous mice exhibited decreased blood glucose levels at 90 and 120 minutes in the GTT. Homozygous mice thus exhibited hypoglycemia in the GTT.

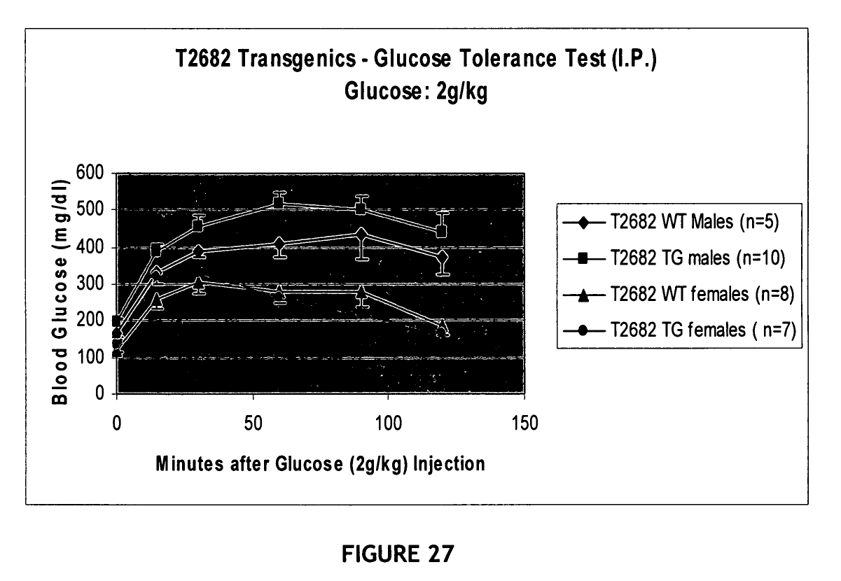

GTT data for transgenic mice overexpressing T243 (TG) compared to wild-type control mice (WT) are graphed in FIG. 27. Transgenic mice overexpressing T243 exhibited increased blood glucose in the GTT, when compared to wild-type control mice. Transgenic mice thus exhibited hyperglycemia in the GTT.

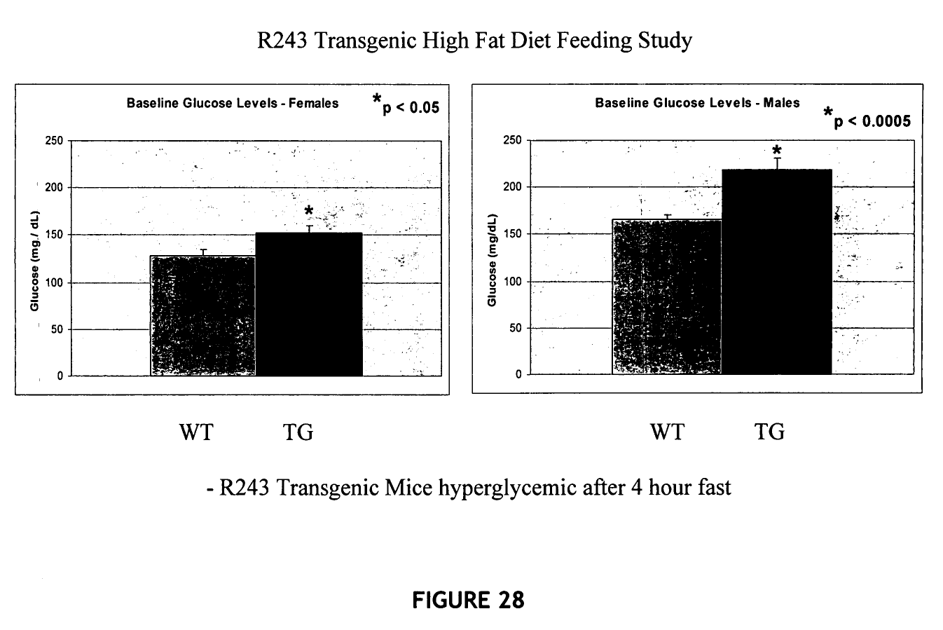

During the HFD, transgenic mice exhibited increased blood glucose levels after a 4 hour fast, when compared to wild-type control mice as shown in FIG. 28. Transgenic mice thus exhibited hyperglycemia upon fasting.

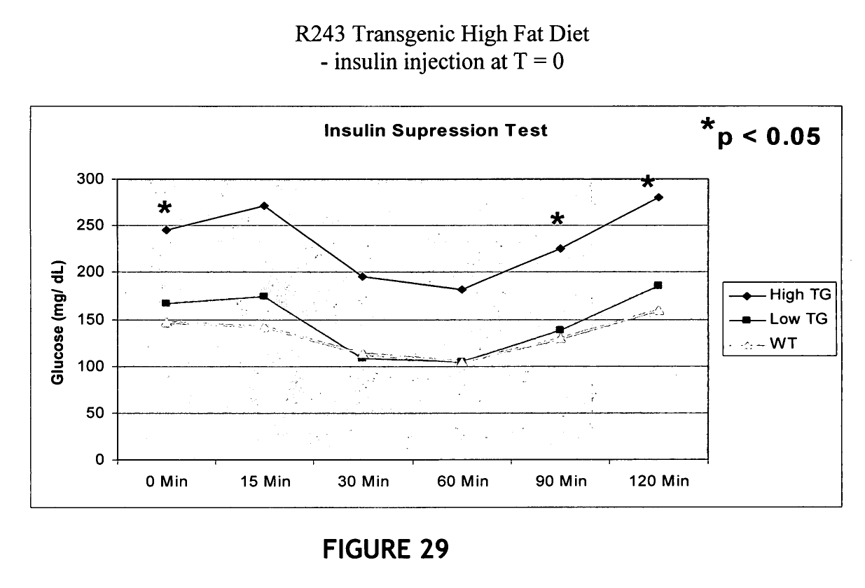

Insulin suppression test (IST). Mice were weighed at time 0 and the basal level of glucose is measured after 5 hour fasting. Insulin (Humulin R, Eli Lilly and Company, Indianapolis, Ind.) is administered intraperitoneally at 0.7 U/kg mouse body weight or otherwise indicated. Tail vein glucose levels are scored at time 15, 30, 60, 90, 120 minutes thereafter. IST data for transgenic mice and wild-type control mice are shown in FIG. 29. The transgenic mice expressing high levels (High TG) of T243 exhibited increased blood glucose levels, when compared to a wild-type control mouse (WT). Although only one mouse was used in each group, the blood glucose differences between the H.E. transgenic mouse compared to the wild-type control mouse were still significant at 0, 90, and 120 minutes. The high expressing transgenic mouse thus exhibited a relatively normal response to i.p. insulin injection but maintained a hyperglycemic state throughout the IST.

Glucose-stimulated insulin secretion (GSIST): Following 5 hour fasting, glucose was administered either intraperitoneally or orally at 2 g/kg mouse body weight. Tail vein blood samples were collected before or 7.5, 15, 30, 60 minutes after the glucose loading. Serum insulin levels were determined by an ELISA kit (Crystal Chem Inc., Chicago, Ill.) with rat insulin standards.

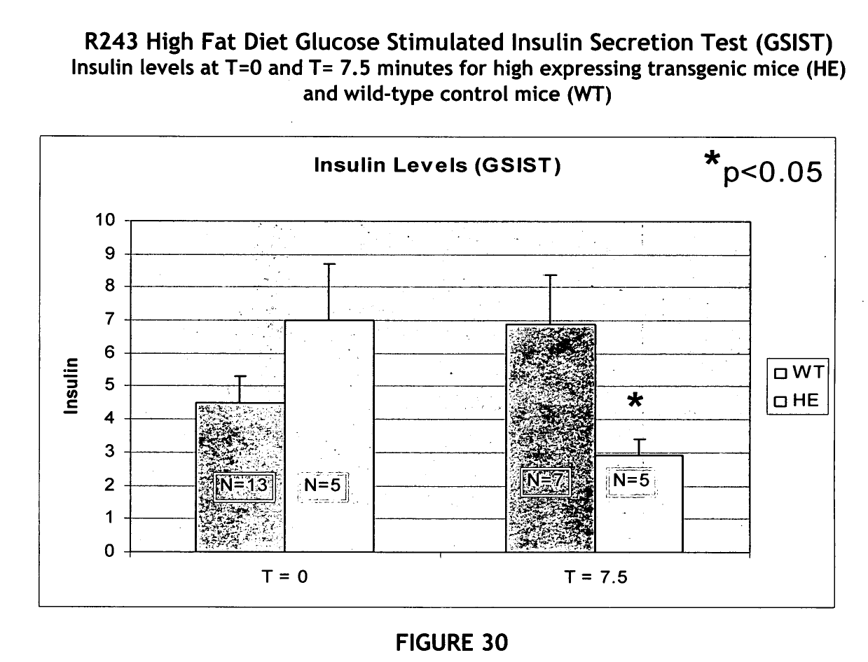

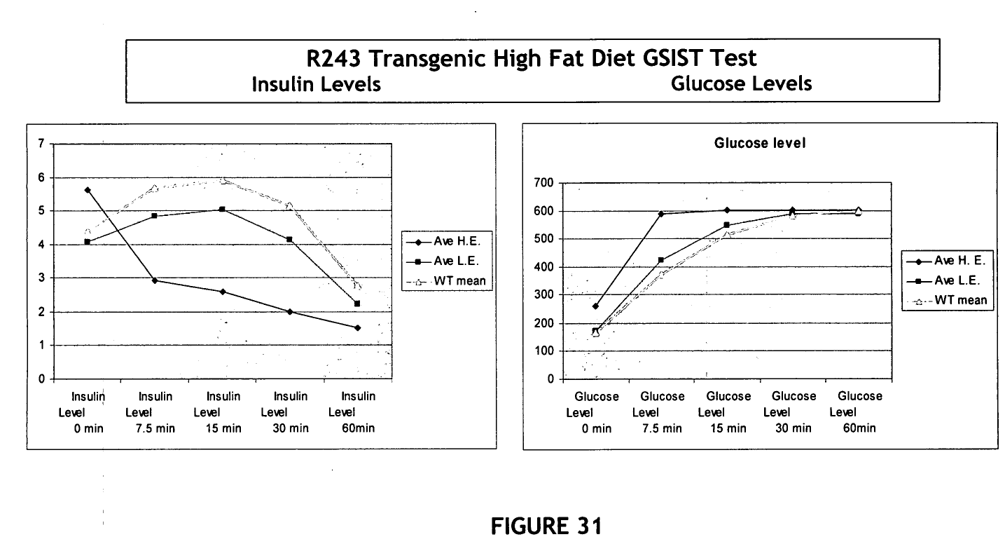

GSIST data are shown in FIG. 30. After high fat diet treatment, high expressing transgenic mice (HE) exhibited increased insulin levels prior to glucose administration, compared to wild-type conntrol mice (WT). After glucose administration at 7.5 minutes, HE mice exhibited significantly decreased insulin levels, compared to wild-type control mice. HE transgenic mice exhibited a rapid decrease in insulin levels following glucose challenge in the GSIST which was sustained over 60 minutes after glucose administration as shown in FIG. 31.

Densitometric Analysis: Mice were anaesthetized with isofluorane and analyzed using a PIXImus™ densitometer, as described above.

Metrics: Body lengths and body weights were recorded right before and during the high fat diet challenge.

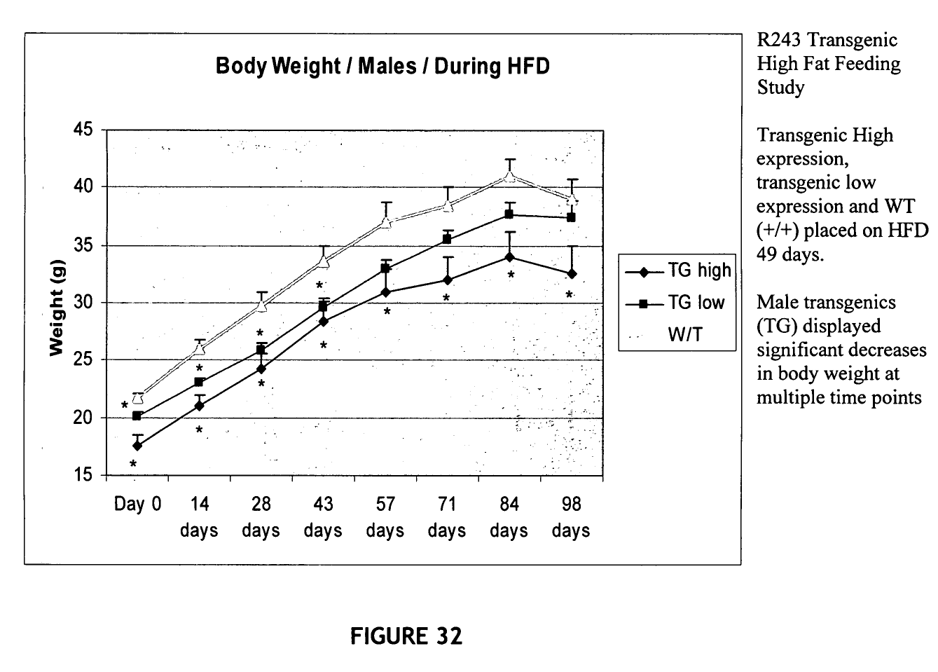

Male T243 transgenic high expressing and low expressing mice and wild-type control mice were subjected to the high fat diet starting at about 49 days of age. Body weights for mice over a 98 day period following the start of the HFD are shown in FIG. 32. At multiple time points throughout the study, male transgenic mice exhibited significantly decreased body weights in the metabolic metrics study when compared to wild-type control mice.

As is apparent to one of skill in the art, various modifications of the above embodiments can be made without departing from the spirit and scope of this disclosure. These modifications and variations are within the scope of this disclosure.

We claim:

1. A transgenic mouse whose genome comprises a homozygous disruption of a trinucleotide repeat protein (TRP) gene, wherein said mouse exhibits a phenotypic abnormality relative to a wild-type control mouse.

2. The transgenic mouse of claim 1, wherein the transgenic mouse exhibits, relative to a wild-type control mouse, at least one physical phenotypic abnormality selected from the group consisting of decreased body length, decreased body weight, decreased body weight to body length ratio, dry skin, decreased spleen weight, decreased spleen weight to body weight ratio, decreased liver weight, decreased kidney weight, decreased thymus weight, abnormal cartilage, reduction of bone formation, shortening of the axial skeleton, shortening of the appendicular skeleton, absence of growth plates in the sternebrae, discontinuous growth plates in the sternebrae, dysplastic changes in the kidney, decreased liver glycogen content, and juvenile lethality.

3. The transgenic mouse of claim 1, wherein the transgenic mouse exhibits, relative to a wild-type control mouse, at least one behavioral phenotypic abnormality selected from the group consisting of hyperactivity, and increased total distance traveled in an open field test.

4. The transgenic mouse of claim 1, wherein the transgenic mouse exhibits, relative to a wild-type control mouse, a phenotypic abnormality comprising at least one change in associated gene expression selected from the group consisting of increased expression of leptin receptor precursor, increased expression of leptin receptor isoform A, increased expression of leptin receptor isoform F, decreased expression of glucose transporter 4 (Glut4) in skeletal muscle, increased expression of insulin-like growth factor (IGF) BP2, increased IGF BP1, and decreased expression of pre-pro-IGF.

5. The transgenic mouse of claim 1, wherein the transgenic mouse exhibits, relative to a wild-type control mouse, at least one hematological phenotypic abnormality selected from the group consisting of increased white blood cells (WBC), increased neutrophils, and increased monocytes.

6. The transgenic mouse of claim 1, wherein the transgenic mouse exhibits, relative to a wild-type control mouse, at least one serum chemistry phenotypic abnormality selected from the group consisting of increased creatinine, decreased calcium (Ca), decreased glucose, increased alkaline phosphatase (ALP), increased alanine aminotransferase (ALT), increased aspartate aminotransferase (AST), increased albumin, decreased globulin, increased total bilirubin (Bil T), increased cholesterol, and increased creatine kinase (CK).

7. The transgenic mouse of claim 1, wherein the transgenic mouse exhibits, relative to a wild-type control mouse, at least one densitometric phenotypic abnormality selected from the group consisting of decreased bone mineral density, decreased bone mineral content, decreased fat tissue mass, and decreased total tissue mass, when compared to wild-type control mice.

8. The transgenic mouse of claim 1, wherein the transgenic mouse exhibits, relative to a wild-type control mouse, a metabolic phenotypic abnormality comprising decreased blood glucose levels in a glucose tolerance test.

9. A transgenic mouse whose genome comprises a heterozygous disruption of a trinucleotide repeat protein (TRP) gene, wherein said mouse exhibits a phenotypic abnormality relative to a wild-type control mouse.

10. The transgenic mouse of claim 9, wherein the transgenic mouse exhibits, relative to a wild-type control mouse, at least one phenotypic abnormality selected from the group consisting of decreased liver weight, increased blood creatinine, increased total distance traveled in the open field test, increased session time in the central zone in the open field test, and increased time immobile in the tail suspension test.

11. A transgenic mouse whose genome comprises one or more additional copies of a TRP gene, wherein said mouse exhibits increased expression of the TRP protein relative to a wild-type control mouse.

12. The transgenic mouse of claim 11, wherein said mouse exhibits a phenotypic abnormality relative to a wild-type control mouse.

13. The transgenic mouse of claim 12, wherein said transgenic mouse exhibits, relative to a wild-type control mouse, at least one phenotypic abnormality selected from the group consisting of increased bone mineral density after estrogen depletion, increased blood glucose in a glucose tolerance test, hyperglycemia upon fasting, hyperglycemic state during an insulin secretion test, decrease in insulin levels following glucose challenge in a glucose-stimulated insulin secretion test, decreased body weights in a metabolic metrics study during a high fat diet.

14. A method of producing the transgenic mouse of claim 1, the method comprising:
a. providing a mouse stem cell comprising a disruption in the endogenous TRP gene;
b. introducing the mouse stem cell into a blastocyst;
c. introducing the blastocyst into a pseudopregnant mouse, wherein the pseudopregnant mouse generates chimeric mice; and
d. breeding said chimeric mice to produce the transgenic mouse.

15. A cell or tissue isolated from the transgenic mouse of claim 1.

16. A targeting construct comprising:
a. a first polynucleotide sequence homologous to at least a first portion of the endogenous TRP gene;
b. a second polynucleotide sequence homologous to at least a second portion of the TRP gene; and
c. a gene encoding a selectable marker located between the first and second polynucleotide sequences.

17. A method of identifying an agent capable of modulating activity of a TRP gene or of a TRP gene expression product, the method comprising:
a. administering a putative agent to the transgenic mouse of claim 1;
b. administering the agent to a wild-type control mouse; and
c. comparing a physiological response of the transgenic mouse with that of the control mouse;
wherein a difference in the physiological response between the transgenic mouse and the control mouse is an indication that the agent is capable of modulating activity of the gene or gene expression product.

18. A transgenic mouse whose genome comprises a disruption in the endogenous TRP gene, wherein said gene encodes for mRNA corresponding to the cDNA sequence of SEQ ID NO: 16, and wherein said disruption comprises replacement of nucleotides 109 to 215 of SEQ ID NO: 16 with a cassette.

19. A transgenic mouse whose genome comprises a null allele of the endogenous TRP gene.
